(19) United States
(12) Patent Application Publication
(10) Pub. No.: US 2017/0233330 A1
(43) Pub. Date: Aug. 17, 2017

(54) Process for the preparation of 3-[(1R,2R)-3-(Dimethylamino)-1-ethyl-2-methylpropyl]-phenol

(71) Applicant: Sandoz AG, Basel (CH)

(72) Inventors: Sven Nerdinger, Kundl (AT); Martin Albert, Prague (CZ); Waldemar Szczepanik, Krakow (PL); Krzysztof Zajac, Krakow (PL); Jörg Salchenegger, Kundl (AT); Joanna Fogt, Krakow (PL)

(73) Assignee: Sandoz AG, Basel (CH)

(21) Appl. No.: 15/503,127

(22) PCT Filed: Aug. 11, 2015
(86) PCT No.: PCT/EP2015/068488

§ 371 Date: Feb. 10, 2017

(30) Foreign Application Priority Data
Aug. 11, 2014 (EP) 14180498.9

(51) Int. Cl. C07C 217/62; C07C 213/02

(52) U.S. Cl. CPC C07C 217/62; C07C 213/02

(57) ABSTRACT

A process for the preparation of a compound of formula (I) and of a acid salt (T) wherein R¹ is selected from the group consisting of alkyl, aryl, cycloalkyl, heteroalkyl, heteroaryl and heterocycloalkyl, R² and R³, are, independently of each other, selected from the group consisting of alkyl, aryl, cycloalkyl, heteroalkyl, heteroaryl and heterocycloalkyl, R⁴, R⁵, R⁶ and R⁷, are independently of each other, selected from the group consisting of H, alkyl, aryl, cycloalkyl, heteroalkyl, heteroaryl and heterocycloalkyl, and wherein the acid salt is a 2,3-Ditoluoyl tartaric acid salt, 2,3-Dibenzoyl tartaric acid salt, 2,3-Dianisoyl tartaric acid salt, 2,3-Dibenzoyl tartaric acid mono(dimethylamide) salt or a mixture of two or more thereof, wherein the tartaric acid salt (T) of the compound of formula (I) contains at least 90% by weight of the tartaric salt of the compound of formula (Ia) based on the total weight of the acid salt of the compound of formula (I).

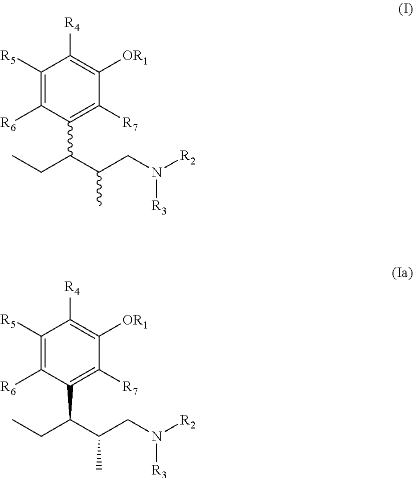

The present invention relates to process for the preparation of a compound of formula (I)

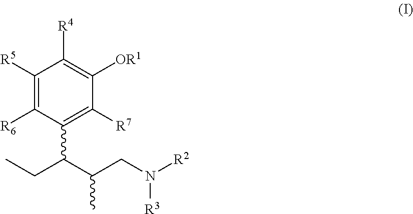

and of an acid salt (T) thereof wherein R¹ is selected from the group consisting of alkyl, aryl, cycloalkyl, heteroalkyl, heteroaryl and heterocycloalkyl, R² and R³, are, independently of each other, selected from the group consisting of alkyl, aryl, cycloalkyl, heteroalkyl, heteroaryl and heterocycloalkyl, R⁴, R⁵, R⁶ and R⁷, are independently of each other, selected from the group consisting of H, alkyl, aryl, cycloalkyl, heteroalkyl, heteroaryl and heterocycloalkyl, and wherein the acid salt (T) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof, wherein the acid salt (T) of the compound of formula (I) contains at least 90% by weight of the salt (T) of the compound of formula (Ia)

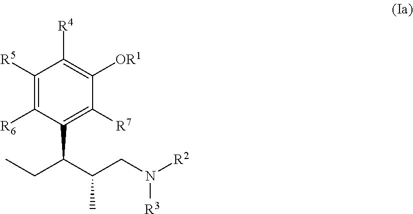

based on the total weight of the acid salt (T) of the compound of formula (I). Further, the present invention relates to use of the compound of formula (I), preferably (Ia) for the preparation of 3-[(1R,2R)-3-(dimethylamino)-1-ethyl-2-methylpropyl (tapentadol) or a pharmaceutically acceptable salt or solvate thereof.

Tapentadol, i.e. 3-[(1R,2R)-3-(dimethylamino)-1-ethyl-2-methylpropyl]phenol, has the structure (XIV) shown hereinbelow:

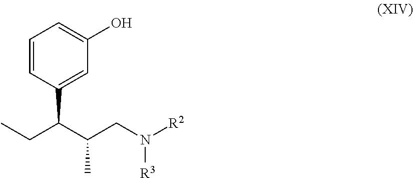

is an analgesic with agonist central action for the receptors μ of the opioids and inhibitor of the re-uptake of noradrenaline, used for the treatment of moderate-to-grave acute pain.

Different methods for the preparation of tapentadol have already been descried in the art. For example, WO 2008/012047 reports the synthesis of tapentadol, starting from 3-bromoanisole which, via organic lithium, is transformed into 3-methoxypropiophenone. A Mannich reaction is carried out on this intermediate which leads a racemic intermediate which is then subjected to an enantiomeric separation by means of reaction with the chiral (2R,3R)-O,O′-dibenzoyltartaric acid. The resolved enantiomer is then alkylated at the carbonyl by means of reaction with ethylmagnesium bromide and finally the product of this reaction is hydrogenated and subsequently demethylated. During the alkylation reaction, the formation of two diastereoisomers is verified; the removal of the undesired isomer (1S,2R) involves the need for crystallization in conditions which lead to the loss of a high percentage of product.

Another synthesis is described in EP 0 693 475 (Buschmann at el.) in example 24 steps 1 to 3. Starting from (2S,3R)-1-dimethylamino-3-(3-methoxyphenyl)-2-methyl-3-pentanol, the conversion of the tertiary hydroxy group into the corresponding chloride via treatment with thionyl chloride and subsequent removal of the chloride via treatment with zinc borohydride, zinc cyanoborohydride or tin cyanoborohydride with or without triphenylphosphine leads to the intermediate compound (2R,3R)-3-(3-methoxyphenyl)-N,N-2-trimethylaminopentanamine which is a precursor of tapentadol. This procedure has the disadvantage of using excessive amounts of thionyl chloride which is an aggressive and corrosive reagent. Furthermore reagents such as zinc borohydride, zinc cyanoborohydride and tin cyanoborohydride do have potentials to present considerable fire and health danger when used on industrial scale. The main disadvantage for the procedure however is the need to separate the stereoisomers via chiral chromatography which is expensive, time consuming and not suitable for any scale up processes.

An alternate process for obtaining tapentadol is described in WO 2004/108658. In this process (2S,38)-1-(dimethylamino)-3-(3-methoxyphenyl)-2-methylpent-3-en, which is produced by treating (3R)-1-dimethylamino-3-(3-methoxyphenyl)-2-methyl-3-pentanol with acid, is converted into a mixture of (2R,3R) (5) and (2R,38)-3-(3-methoxyphenyl)-N,N-2-trimethylaminopentanamine. These diastereomeres are then separated from each other. Subsequently, the final deprotection with HBr leads to tapentadol. The main disadvantage of this process is the separation of the stereoisomers at very late stage of the process. This leads to a very low overall yield which renders the synthesis disadvantageous for any scale up processes.

An alternate process for obtaining tapentadol is described in WO 2011/067714 by converting(S)-3-(dimethylamino)-2-methyl-1-(3-nitrophenyl)-propan-1-one to (S)-3-(dimethylamino)-2-methyl-1-(3-aminophenyl)-propan-1-one via reduction with Sn/HCl. This reduction, however, is not suitable for large-scale production. The thus obtained compound is then further transformed into (2R,3R)-3-(3-Aminophenyl)-1-(dimethylamino)-2-methyl-pentan-3-ol by addition of ethyl magnesium bromide. After separation of the thereby obtained diastereomeres, (2R,3R)-3-(3-Aminophenyl)-1-(dimethylamino)-2-methyl-pentan-3-ol is converted in a three-step process into (2R,3R)dimethyl-[2-methyl-3-(3-aminophenyl)-pentylamine. The desired product tapentadol is finally obtained by a final treatment with NaNO2 in the presence of sulfuric acid/water. Besides the not defined ratio of stereoisomers obtained after the addition of EtMgBr, the synthesis is in particular disadvantagoues in that toxic, corrosive and expensive reagents like Sn/HCl and (CF₃CO₂)₂O are employed.

An alternative process for obtaining tapentadol is described in WO 2011/080736. In the described process, 3-methoxyphenylpropan-1-one is converted to 3-(3-methoxyphenyl)-N,N,2-trimethylpent-2-enamide via reaction with bis(ethyl)-1-(dimethylamino)-1-oxopropan-2ylphosphonate using NaH as base which is a difficult reagent to handle on large scale. The resulting compound is obtained as a mixture of E- and Z-isomers in a ratio of 28:72. This compound is then further transformed into (2R,3R)-3-(3-methoxyphenyl)-N,N,2-trimethylpentanamide in an asymmetric hydrogenation step using a Ru catalyst chiral ligands to give (2R,3R)-3-(3-methoxyphenyl)-N,N,2-trimethylpentanamide in >97 ee. This compound is then reduced and deprotected to give tapentadol (1). The major disadvantage is the use of Ru as expensive precious metal and expensive chiral ligands since I the hydrogenation step very high amounts of these compounds are needed.

Thus, there is still the need for advantageous synthetic procedures for the preparation of tapentadol.

Surprisingly it has been found that tapentadol may be advantageously and in particular cost effectively be prepared in high yields with the processes according to the present invention i.e. the preparation of a compound of formula (I) which are hereinafter identified as Method 1 and Method 2. Advantageously the methods according to the present invention allow introducing the molecule chirality of compound (I) in the last step of the synthesis by reducing the double bond C═C. The reduction process can be highly controlled by choosing the suitable reducing agent and/chiral auxiliary. The stereopurity (enantio- and/or diastereo-purity) may further be improved by chiral resolution e.g by formation of acid chiral salt as those disclosed hereinafter or via other suitable method such as chiral chromatography.

Thus the present invention provides a process (Method 1) for the preparation of a compound of formula (I)

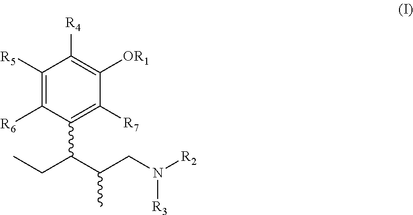

the process comprising
(a1) providing a compound of formula (II)

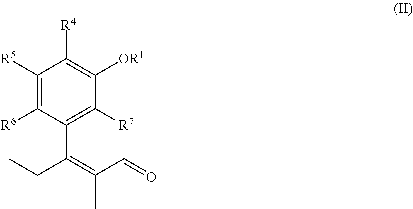

(a2) reacting the compound of formula (II) with an amine HN(R²)(R³) and reducing the resulting reaction product with hydrogen in the presence of a catalyst, preferably in the presence of Pd/C,
to give the compound of formula (I).

Thus the present invention provides a process (Method 2) for the preparation of a compound of formula (I)

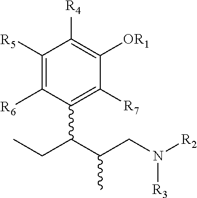

the process comprising
(aa) providing a compound of formula (IX)

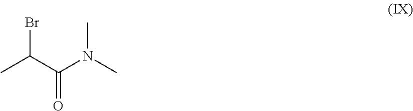

(ab) providing a compound of formula (X)

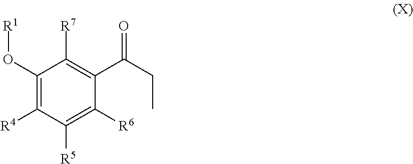

(ac) reacting the compound of formula (IX) with the compound of formula (X) in the presence of Zn to give a compound of formula (XI)

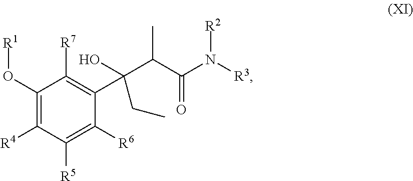

(ad) dehydrating the compound of formula (XI) to give a compound of formula (XIIa)

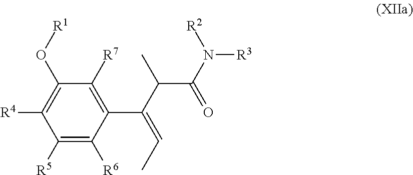

and/or a compound of formula (XIIb)

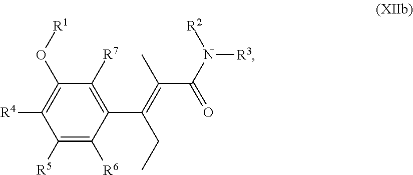

(ae) reducing the compound of formula (XIIa) and/or (XIIb),
to give the compound of formula (I).

Thus the processes according to Methods 1 and 2 may further comprise the preparation and the subsequent isolation of a chiral acid salt, in particular a tartaric acid salt, of the compound shown below from a diastereomeric mixture comprising said compound.

Thus, the present invention provides a process for the preparation of an acid salt (T), preferably a tartaric acid salt (T), of a compound of formula (I)

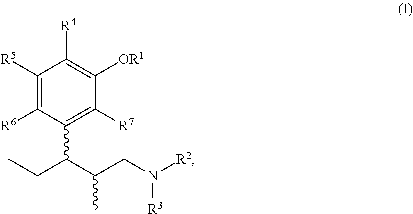

preferably

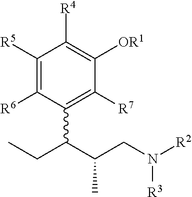

wherein R¹ is selected from the group consisting of alkyl, aryl, cycloalkyl, heteroalkyl, heteroaryl and heterocycloalkyl, R² and R³, are, independently of each other, selected from the group consisting of alkyl, aryl, cycloalkyl, heteroalkyl, heteroaryl and heterocycloalkyl, R⁴, R⁵, R⁶ and R⁷, are independently of each other, selected from the group consisting of H, alkyl, aryl, cycloalkyl, heteroalkyl, heteroaryl and heterocycloalkyl, and wherein the acid salt (T) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof, and wherein the process comprises
(a) providing a compound of formula (I)

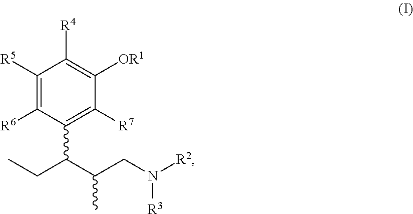

preferably

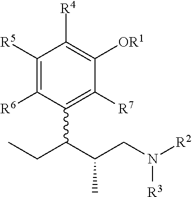

comprising a diastereomeric mixture of the compounds of formula (Ia) and formula (Ib),

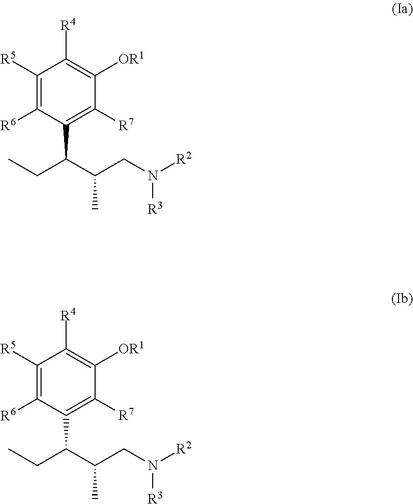

(b) forming an acid salt (T*), preferably a tartaric acid salt (T*), of at least part of the compound of formula (I) by treating the compound of formula (I) with a chiral acid, preferably a single stereoisomer of a chiral acid, more preferably with a tartaric acid, more preferably a single stereoisomer of a tartaric acid, wherein the tartaric acid is preferably selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and a mixture of two or more thereof, in a suitable solvent, and precipitating, preferably crystallizing, at least part of the acid salt (T*) formed, thereby obtaining a mixture comprising the precipitated, preferably crystallized acid salt (T) and the solvent;
(c) preferably separating the precipitated, preferably crystallized, acid salt (T) of the compound of formula (I) from the mixture obtained in (b),
   wherein the acid salt (T) of the compound of formula (I) contains at least 90% by weight of the salt of the compound of formula (Ia) based on the total weight of the acid salt (T) of the compound of formula (I).

Further, the present invention relates to an acid salt obtained or obtainable by said process.

Further, the present invention relates to the acid salt (T) of a compound of formula (I) and to the compound of formula (I) as such

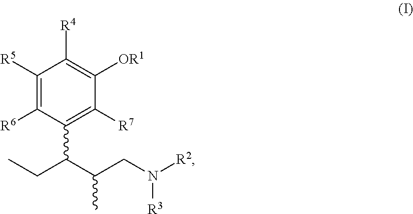

preferably

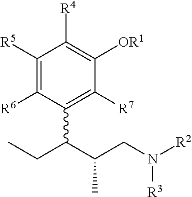

wherein R¹ is selected from the group consisting of alkyl, aryl, cycloalkyl, heteroalkyl, heteroaryl and heterocycloalkyl, R² and R³, are, independently of each other, selected from the group consisting of alkyl, aryl, cycloalkyl, heteroalkyl, heteroaryl and heterocycloalkyl, R⁴, R⁵, R⁶ and R⁷, are independently of each other, selected from the group consisting of H, alkyl, aryl, cycloalkyl, heteroalkyl, heteroaryl and heterocycloalkyl, and wherein the acid salt (T) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof, wherein the acid salt (T) of the compound of formula (I) contains at least 90% by weight of the acid salt of the compound of formula (Ia) based on the total weight of the acid salt (T) of the compound of formula (I).

Further, the present invention relates to a compound of formula (II)

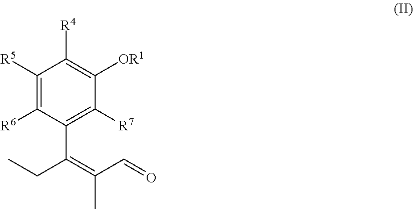

wherein R¹ is selected from the group consisting of alkyl, aryl, cycloalkyl, heteroalkyl, heteroaryl and heterocycloalkyl, R⁴, R⁵, R⁶ and R⁷, are independently of each other, selected from the group consisting of H, alkyl, aryl, cycloalkyl, heteroalkyl, heteroaryl and heterocycloalkyl, as well as to a compound of formula (III)

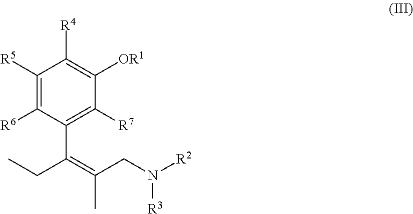

wherein R¹ is selected from the group consisting of alkyl, aryl, cycloalkyl, heteroalkyl, heteroaryl and heterocycloalkyl, R² and R³, are, independently of each other, selected from the group consisting of alkyl, aryl, cycloalkyl, heteroalkyl, heteroaryl and heterocycloalkyl, R⁴, R⁵, R⁶ and R⁷, are independently of each other, selected from the group consisting of H, alkyl, aryl, cycloalkyl, heteroalkyl, heteroaryl and heterocycloalkyl.

Further, the present invention relates to a compound of formula

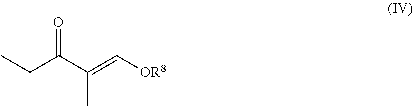

wherein R⁸ is selected from the group consisting of methyl, ethyl, isopropyl, ter.-butyl and —CH(isopropyl)₂, preferably wherein R⁸ is —CH(isopropyl)₂.

Additionally, the present invention provides a process for the preparation of a compound of formula (XIV)

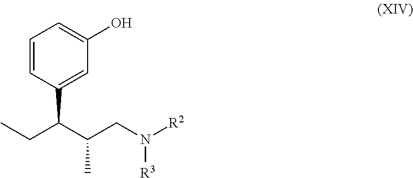

or a pharmaceutically acceptable salt or solvate thereof, the process comprising
(i) providing an acid salt (T) of a compound of formula (I) as described above, wherein R⁴, R⁵, R⁶ and R⁷ are H,
(ii) transforming the group —OR¹ to OH,
(iii) optionally purifying the compound obtained in (ii)
   to give the compound of formula (XIV)
(iv) optionally preparing pharmaceutically acceptable salt or solvate of compound (XIV) of (ii) or (iii).

### The Compound of Formula (I)

As described above, the compound of formula (I) comprises of a diastereomeric mixture of the compounds of formula (Ia) and formula (Ib)

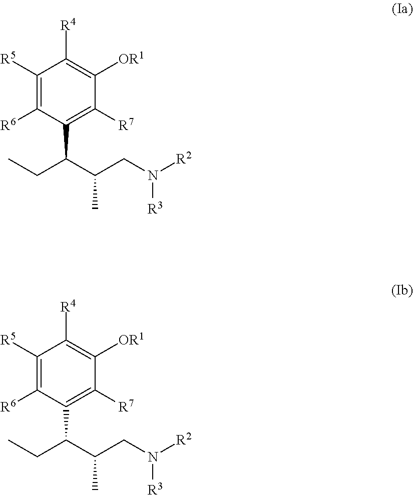

Besides, compound (I) may optionally further comprise the compound of formula (Ic) and/or the compound of formula (Id).

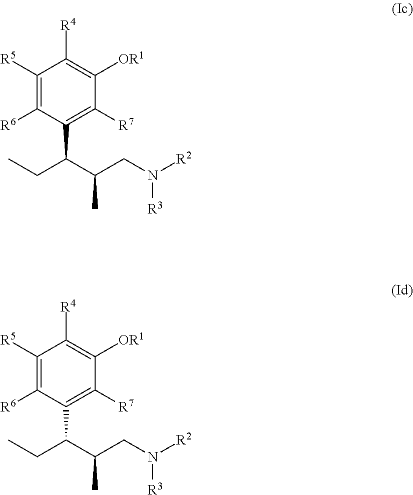

Preferably compound (I) consist of a diastereomeric mixture of the compounds of formula (Ia) and formula (Ib), optionally the compound of formula (Ic), and optionally the compound of formula (Id).

The compound of formula (I) provided in step (a) contains the compound of formula (Ia) e.g. in an amount in the range of from 1 to 80% by weight, such as in the range of from 10 to 70% by weight, or in the range of from 30 to 60% by weigh, or in the range of from 45 to 55% by weight, based on the total weight of the compound of formula (I), i.e. the sum of compound (Ia) and (Ib). Thus, the compound of formula (I) contains the compound of formula (Ib) e.g. in an amount in the range of from 20 to 99% by weight, such as in the range of from 30 to 90% by weight, or in the range of from 40 to 70% by weigh, or in the range of from 45 to 55% by weight, based on the total weight of the compound of formula (I), i.e. the sum of compound (Ia) and (Ib), optionally including (Ic) and optionally including (Id).

Preferably, the compound of formula (I) contains the compound of formula (Ia) in an amount of from 30 to 90% by weight, and the compound of formula (Ib) in an amount of from 10 to 30%, based on the total weight of the compound of formula (I).

More preferably, the compound of formula (I) in (a) consists of a diastereomeric mixture of compounds of formula (Ia) and formula (Ib), the mixture comprising the compounds of formula (Ia) in a molar ratio in the range of from 0.2 to 1.2, preferably 0.4 to 1.

R⁴, R⁵, R⁶ and R⁷, are independently of each other, selected from the group consisting of H, alkyl, aryl, cycloalkyl, heteroalkyl, heteroaryl and heterocycloalkyl.

Within the meaning of the present invention, the term “alkyl” relates to non-branched alkyl residues as well as to branched alkyl residues. The term also encompasses alkyl groups which are further substituted by one or more suitable substituents. The term “substituted alkyl” as used in this context of the present invention preferably refers to alkyl groups being substituted in any position by one or more substituents, preferably by 1, 2, 3, 4, 5 or 6 substituents, more preferably by 1, 2, or 3 substituents. If two or more substituents are present, each substituent may be the same or may be different from the at least one other substituent. There are in general no limitations as to the substituent. The substituents may be, for example, selected from the group consisting of aryl, alkenyl, alkynyl, aryl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl, ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups and carboxyl groups. The term “alkenyl” as used in the context of the present invention refers to unsaturated alkyl groups having at least one double bond. The term also encompasses alkenyl groups which are substituted by one or more suitable substituents. The term “alkynyl” refers to unsaturated alkyl groups having at least one triple bond. The term also encompasses alkynyl groups which are substituted by one or more suitable substituents.

The term “heteroalkyl” refers to, optionally suitably substituted, alkyl groups comprising one or more heteroatoms or functional groups, such as, by way of example, —O—, —S—, —NH—, —NH—C(═O)—, —C(═O)—NH—, and the like.

The term cycloalkyl refers to, but is not limited to, optionally suitably substituted non aromatic monocyclic hydrocarbon groups containing from 3 to 7 ring carbon atoms, such as cyclopropyl, cyclobutyl, cyclopentyl, cyclohexyl or cycloheptyl, more particularly cyclopropyl. In addition, the term “cycloalkyl” also encompasses multicyclic hydrocarbon groups such as cyclic groups containing from 3 to 10 carbon atoms. Bicyclic rings consisting of two saturated carbocycles having one or more carbon atoms in common. Examples for bicyclic cycloalkyl are bicyclo[2.2.1]heptanyl or bicyclo[2.2.2]octanyl.

The term “heterocycloalkyl” refers to, optionally suitably substituted, cycloalkyl groups comprising one or more heteroatoms or functional groups, such as, by way of example, —O—, —S—, —NH—, —NH—C(═O)—, —C(═O)—NH—, and the like.

Within the meaning of the present invention, the term “aryl” refers to, but is not limited to, optionally suitably substituted 5- and 6-membered single-ring aromatic groups as well as optionally suitably substituted multicyclic groups, for example bicyclic or tricyclic aryl groups. The term “aryl” thus includes, for example, optionally substituted phenyl groups or optionally suitably substituted naphthyl groups. Aryl groups can also be fused or bridged with alicyclic or heterocycloalkyl rings which are not aromatic so as to form a polycycle, e.g., benzodioxolyl or tetraline.

The term “heteroaryl” as used within the meaning of the present invention includes optionally suitably substituted 5- and 6-membered single-ring aromatic groups as well as substituted or unsubstituted multicyclic aryl groups, for example tricyclic or bicyclic aryl groups, comprising one or more, preferably from 1 to 4 such as 1, 2, 3 or 4, heteroatoms, wherein in case the aryl residue comprises more than 1 heteroatom, the heteroatoms may be the same or different. Such heteroaryl groups including from 1 to 4 heteroatoms are, for example, benzodioxolyl, pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthyridinyl, quinolinyl, isoquinolinyl, indolyl, benzofuranyl, purinyl, deazapurinyl, or indolizinyl.

The terms “optionally substituted cycloalkyl” and “optionally substituted aryl” and the term “optionally substituted heteroaryl” as used in the context of the present invention describes moieties having substituents replacing a hydrogen on one or more atoms, e.g. C or N, of an aryl or heteroaryl moiety. Again, there are in general no limitations as to the substituent. The substituents may be, for example, selected from the group consisting of alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups.

Preferably, R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl.

More preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl.

More preferably, R⁴, R⁵, R⁶ and R⁷ are H.

Thus, the present invention also relates to processes, as described above, wherein R⁴, R⁵, R⁶ and R⁷ are H.

In particular the present invention relates to a compound of formula (I), as described above, and, wherein R⁴, R⁵, R⁶ and R⁷ are H and the wherein the compound of formula (I) thus has the structure:

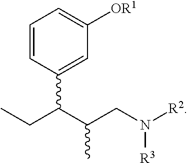

As described above, residue R¹ is selected from the group consisting of alkyl, aryl, cycloalkyl, heteroalkyl, heteroaryl and heterocycloalkyl. Preferably, R¹ is selected from the group consisting of methyl, ethyl, propyl, isopropyl and benzyl.

More preferably, R¹ is methyl.

Thus, the present invention also relates to a process, as described above, wherein R¹ is methyl, more preferably, wherein R¹ is methyl and wherein R⁴, R⁵, R⁶ and R⁷ are H and the wherein the compound of formula (I) thus has the structure:

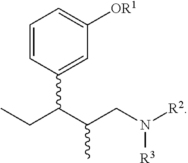

As described above, R² and R³, are, independently of each other, selected from the group consisting of alkyl, aryl, cycloalkyl, heteroalkyl, heteroaryl and heterocycloalkyl. Preferably, R² and R³, are both alkyl groups, preferably, independently of each other, selected from the group consisting of methyl, ethyl, propyl and isopropyl, more preferably, R² and R³, are methyl.

Thus, the present invention also relates to processes, as described above, wherein R² and R³, are, more preferably, wherein R² and R³, methyl and wherein R⁴, R⁵, R⁶ and R⁷ are H and the wherein the compound of formula (I) thus has the structure:

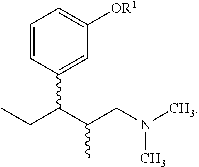

A bond shown hereinunder and above as “ [symbol] ”, is denoted to represent a single bond, wherein the relative orientation of the substituents of this single bond is not defined. Thus, a structure including the bond “ [symbol] ” encompasses both, the R as well as the S orientation.

### Step (b)

In step (b), at least part of the compound of formula (I) provided in step (a) is transformed into the corresponding acid salt (T*). The acid salt (T*) contains the acid salt of the compound of formula (Ia) e.g. in an amount in the range of from 1 to 80% by weight, such as in the range of from 10 to 70% by weight, or in the range of from 30 to 60% by weigh, or in the range of from 45 to 55% by weight, based on the total amount of the acid (T*). Subsequently at least part of the acid salts (T*) obtained are precipitated, preferably crystallized. This is preferably achieved, by contacting (treating) the compound of formula (I) in a suitable solvent with the chiral acid, preferably a single stereoisomer of the chiral acid, more preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof. Thereby a mixture comprising the crystallized acid salt (T) of the compound of formula (I) and the solvent is formed. As mentioned above, the precipitated, preferably crystallized acid salt (T) of the compound of formula (I) contains at least 90% by weight of the acid salt of the compound of formula (Ia) based on the total weight of the acid salt (T) of the compound of formula (I).

It is to be noted that the mixture obtained in step (b) may comprise further compounds, in particular non crystalline forms of the compound of formula (I) and acid salts thereof. Preferably, the mixture obtained in (b) comprises non crystalline forms of the compound of formula (Ib) and salts thereof, more preferably, non-crystalline forms of the compound of formula (I) and salts thereof.

The acid salt (T*) of the compound of formula (I) is denoted to encompass all acid salts of compound (I), preferably all the stereoisomers of the compound of formula (I) formed in step (b) including, the acid salt (T) which precipitates as well as all acid salts formed which remain dissolved, thus the acid salt (T*) may comprise a mixture of acid salt of compounds of formula (Ia) and (Ib), optionally additionally of (Ic) and/or (Id).

Preferably in step (b) and (c) of the invention, a chiral resolution of the stereoisomers (Ia) and (Ib) is carried out, wherein preferably (Ia) is also separated from (Ic) and (Id) if present.

As to the solvent used in step (b), any suitable organic solvent in which the compound of formula (I) is sufficiently soluble may be used. In particular, the solvent is selected from the group consisting of ethyl acetate, acetone, isopropanol, methanol, water, formic acid ethyl ester, isopropyl acetate, propyl acetate, butyl acetate and mixtures of two or more thereof. More preferably, the suitable solvent comprises ethyl acetate, preferably is ethyl acetate.

It is to be understood that in step (b) a further solvent may be added in order to precipitate, preferably crystallize, the compound of acid salt (T). In this case, the mixture obtained in b) preferably additionally comprises the further solvent.

This further solvent may be added prior to, together with or after the addition of the acid to the compound of formula (I). According to a preferred embodiment, the compound of formula (I) is dissolved in the suitable solvent mentioned above and a mixture, preferably a solution of the acid, in a further solvent is added to the solution, wherein the further solvent and the suitable solvent may be the same or may be different.

In particular, the further solvent is selected from the group consisting of ethyl acetate, acetone, isopropanol, methanol, water, formic acid ethyl ester, isopropyl acetate, propyl acetate, butyl acetate and mixtures of two or more thereof. More preferably, the further solvent comprises ethyl acetate, preferably is ethyl acetate.

More preferably, the suitable solvent and the further solvent are the same, in particular, the both comprise ethyl acetate, preferably both are ethyl acetate.

Thus, the present invention also relates to a process for the preparation of an acid salt (T) of a compound of formula (I), as described above, and an acid salt (T) of compound of formula (I), obtained or obtainable by said process, wherein step (b) comprises dissolving the compound of formula (I) in the suitable solvent and adding a solution of the acid dissolved in a further solvent to the solution, wherein the further solvent and the suitable solvent are preferably the same, more preferably ethyl acetate.

Preferably, the compound of formula (I) is dissolved in the suitable solvent and the mixture is heated to a temperature in the range of from 30 to 80° C., more preferably to a temperature in the range of from 30 to 60° C., more preferably to a temperature in the range of from 30 to 55° C., to a temperature in the range of from 30 to 50° C., prior to the addition of the acid. During the heating step, the temperature may be varied, constantly or stepwise, or held essentially constant. Preferably, the mixture is heated until a clear solution of the compound of formula (I) in the suitable solvent is obtained. Optionally, the mixture is afterwards cooled to room temperature.

The precipitation, preferably the crystallizing, in (b) is preferably carried out at a temperature in the range of from 0 to 60° C., wherein the temperature is preferably continuously or stepwise decreased during step b). The acid may thus e.g. be added to a solution of the compound of formula (I) in the suitable solvent, which has been previously heated or which has been previously heated and afterwards cooled to a specific temperature, or which has not been previously heated.

After the addition of the acid, and optionally the further solvent, the mixture may again be heated or alternatively be cooled or the temperature may be held constant. Preferably, the mixture is cooled to a temperature in the range of from −10° C. to 55° C., more preferably to a temperature in the range of from 0 to 55° C., more preferably to a temperature in the range of from 0 to 50° C., to a temperature in the range of from 30 to 40° C., more preferably to a temperature in the range of from 0 to 25° C.

Preferably, the mixture obtained in b) consists of the acid salt (T), optionally unreacted acid, optionally unreacted compound of formula (I), optionally further acid salts (salt (T*) minus amount of precipitated tartaric acid (T)), the suitable solvent and optionally the further suitable solvent.

### The Acid Salt (T) of the Compound of Formula (I)

The acid salt (T), which precipitates, preferably crystallizes, in step (b), contains at least 95% by weight, more preferably at least 96% by weight, more preferably at least 97% by weight, more preferably at least 98% by weight, more preferably at least 99% by weight, more preferably at least 99.5% by weight, more preferably at least 99.9% by weight, of the tartaric salt of the compound of formula (Ia), based on the total weight of the acid salt (T) of the compound of formula (I).

It is to be understood that most preferably, a single stereoisomer of the acid is employed. The term “single stereoisomer of a chiral acid” in this context is denoted to mean that the chiral acid comprises less than 1% by weight, preferably less than 0.5% by weight, more preferably less than 0.1% by weight, more preferably less than 0.05% by weight, more preferably less than 0.01% by weight, more preferably essentially no, more preferably no impurities of respective other stereoisomers of the chiral acid, based on the total weight of the chiral acid.

The chiral acid according to the present invention is any chiral acid suitable to prepare two diastereoisomers of compound (A) having different solubility. More preferably the chiral acid is a tartaric acid, as mentioned above.

In case the chiral acid is a tartaric acid being a mixture of two or more of 2,3-Ditoluoyl tartaric acid, 2,3-Dibenzoyl tartaric acid, 2,3-Dianisoyl tartaric acid and 2,3-Dibenzoyl tartaric acid mono(dimethylamide), respectively, this means that of each of the chiral acid derivatives within the mixture only a single stereoisomer is present

Preferably, the acid salt (T) of formula (I) consists of the acid salt of the compound of formula (Ia)

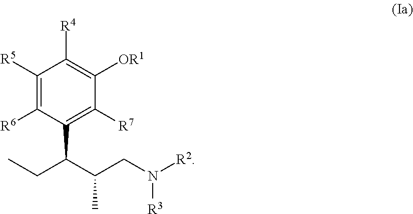

The acid employed in step b) is preferably selected from the group consisting of 2,3-Ditoluoyl tartaric acid salts, 2,3-Dibenzoyl tartaric acid salts, 2,3-Dianisoyl tartaric acid salts, 2,3-Dibenzoyl tartaric acid mono(dimethylamide) salts and mixture of two or more thereof. More preferably, the acid compound is a 2,3-Dibenzoyl tartaric acid or a 2,3-Ditoluoyl tartaric acid.

It is to be understood that most preferably, a single stereoisomer of the acid is employed, thus a single stereoisomer of a tartaric acid selected from the group consisting of 2,3-Ditoluoyl tartaric acid salts, 2,3-Dibenzoyl tartaric acid salts, 2,3-Dianisoyl tartaric acid salts, 2,3-Dibenzoyl tartaric acid mono(dimethylamide) salts and mixture of two or more thereof, more preferably a single stereoisomer of 2,3-Dibenzoyl tartaric acid or a 2,3-Ditoluoyl tartaric acid.

The term “single stereoisomer of a tartaric acid” in this context is denoted to mean that the acid comprises less than 1% by weight, preferably less than 0.5% by weight, more preferably less than 0.1% by weight, more preferably less than 0.05% by weight, more preferably less than 0.01% by weight, more preferably essentially no, more preferably no impurities of respective other stereoisomers of the acid, based on the total weight of the acid.

In particular L-2,3-Dibenzoyl tartaric acid (=DBTA or (−)DBTA) or L-2,3-Ditoluoyl tartaric acid (LTTA) is employed, most preferably DBTA.

Thus, the acid salt is preferably the salt of 2,3-Ditoluoyl tartaric acid salts, 2,3-Dibenzoyl tartaric acid salts, 2,3-Dianisoyl tartaric acid salts, 2,3-Dibenzoyl tartaric acid mono(dimethylamide) salts or comprises a mixture of salts of two or more of the aforementioned acids.

As mentioned, above, preferably, R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl.

More preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl.

More preferably, R⁴, R⁵, R⁶ and R⁷ are H.

Thus, the present invention also relates to a process, as described above, and to an acid salt, preferably a tartaric acid (T) salt, as described above, as well as to an acid salt (T), preferably a tartaric acid (T) obtained or obtainable by said process, wherein R⁴, R⁵, R⁶ and R⁷ are H.

In particular the present invention also relates to a process, as described above, and to an acid salt, preferably a tartaric acid (T) salt, as described above, as well as to an acid salt (T), preferably a tartaric acid (T) obtained or obtainable by said process, wherein the compound of formula (I) has the structure:

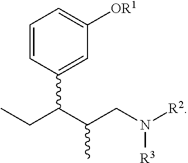

As described above, residue R¹ is selected from the group consisting of alkyl, aryl, cycloalkyl, heteroalkyl, heteroaryl and heterocycloalkyl. Preferably, R¹ is selected from the group consisting of methyl, ethyl, propyl, isopropyl and benzyl.

More preferably, R¹ is methyl.

Thus, the present invention also relates to a process, as described above, and to an acid salt, preferably a tartaric acid (T) salt, as described above, as well as to an acid salt (T), preferably a tartaric acid (T), obtained or obtainable by said process, wherein R¹ is methyl and wherein R⁴, R⁵, R⁶ and R⁷ are H and the wherein the compound of formula (I) thus has the structure:

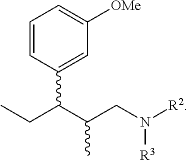

As described above, R² and R³, are, independently of each other, selected from the group consisting of alkyl, aryl, cycloalkyl, heteroalkyl, heteroaryl and heterocycloalkyl. Preferably, R² and R³, are both alkyl groups, preferably, independently of each other, selected from the group consisting of methyl, ethyl, propyl and isopropyl, more preferably, R² and R³, are methyl.

Thus, the present invention also relates to a process, as described above, as described above, and to an acid salt, preferably a tartaric acid (T) salt, as described above, as well as to an acid salt (T), preferably a tartaric acid (T) obtained or obtainable by said process, wherein R² and R³, are, more preferably, wherein R² and R³, methyl and wherein R⁴, R⁵, R⁶ and R⁷ are H and the wherein the compound of formula (I) thus has the structure:

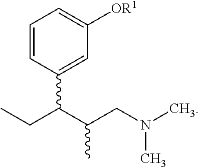

### Step c)

In the optional step c) of the process of the invention, the acid salt (T) is separated from the mixture obtained in b).

Preferably, the process of the invention comprises the steps (a), (b) and (c), mentioned above.

Thus, the present invention also relates to a process, as described above, and to an acid salt (T), as described above, the process comprising
(a) providing a compound of formula (I)

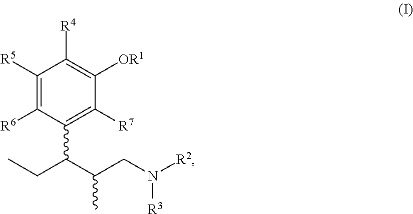

preferably

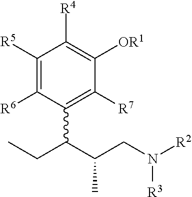

comprising a diastereomeric mixture of the compounds of formula (Ia) and formula (Ib)

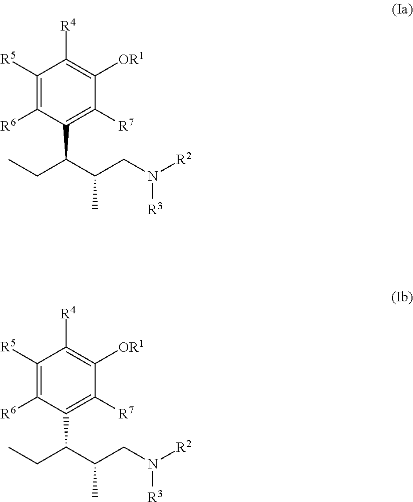

(b) forming an acid salt, preferably a tartaric acid salt, (T*) of at least part of the compound of formula (I) by treating the compound of formula (I) with a chiral acid, preferably a single stereoisomer of a chiral acid, more preferably with a tartaric acid, more preferably a single stereoisomer of a tartaric acid, wherein the tartaric acid is preferably selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and a mixture of two or more thereof, in a suitable solvent, and precipitating, preferably crystallizing, at least part of the acid salt (T*) formed, thereby obtaining a mixture comprising the precipitated, preferably crystallized acid salt (T) and the solvent;
(c) separating the precipitated, preferably crystallized, acid salt (T) of the compound of formula (I) from the mixture obtained in (b),
   wherein the acid salt (T) of the compound of formula (I) contains at least 90% by weight of the acid salt of the compound of formula (Ia) based on the total weight of the acid salt (T) of the compound of formula (I).

The separation may be carried by any suitable method known to those skilled in the art. Preferably, the separating in (c) is carried out by centrifugation or filtration, preferably filtration.

It is to be understood that the separated salt may be subjected to a further treatment such as an after-treatment like such as a purification step and/or lyophilization.

### Step a)

In step a) according to the invention the compound of formula (I) is provided. This includes any possible provision, such as any possible synthesis of the compound of formula (I).

According to a preferred embodiment, step a) comprises providing a compound of formula (II)

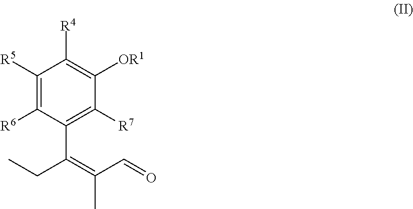

and reacting the compound of formula (II) with an amine HN(R²)(R³) and reducing the resulting reaction product to give, optionally after further steps, the compound of formula (I).

The present invention thus also relates to a compound of formula II, a process for the preparation of a compound of formula (II), and a compound of formula (II) obtained or obtainable by said process, the compound having the structure

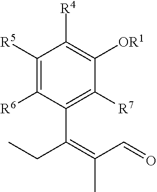

wherein R¹ is selected from the group consisting of alkyl, aryl, cycloalkyl, heteroalkyl, heteroaryl and heterocycloalkyl,

R⁴, R⁵, R⁶ and R⁷, are independently of each other, selected from the group consisting of H, alkyl, aryl, cycloalkyl, heteroalkyl, heteroaryl and heterocycloalkyl.

The bond shown as “═” in the formula (II) above, is denoted to represent a double bond, wherein the relative orientation of the substituents of this double bond is not defined. Thus, a structure including the bond “═” encompasses the E as well as the Z isomer as well as mixtures thereof.

For the compound according to formula (II) shown above, this means, that the compound of formula II has the structure (IIa) or (IIb) or consists of a mixture of (IIa) and (IIb)

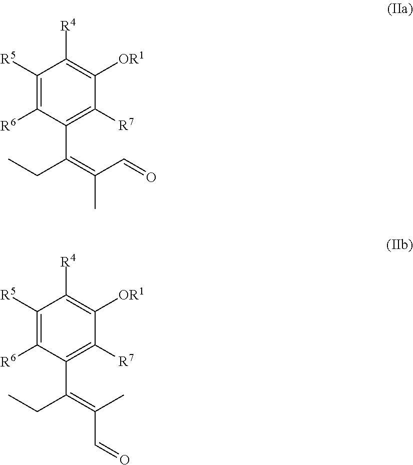

Preferably, the compound of formula (II) consists of 0 to 100% by weight of the compound of formula (IIa) and 100 to 0% by weight of the compound of formula (IIb), more preferably of 75 to 25% by weight of the compound of formula (IIa) and 50 to 50% by weight of the compound of formula (IIb), more preferably of 0 to 100% by weight of the compound of formula (IIa) and 25 to 75% by weight of the compound of formula (IIb), more preferably of 50% by weight.

As already mentioned above with respect to the compound of formula (I), also in the compound of formula (II) R⁴, R⁵, R⁶ and R⁷, are independently of each other, selected from the group consisting of H, alkyl, aryl, cycloalkyl, heteroalkyl, heteroaryl and heterocycloalkyl. Preferably, R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl. More preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl.

More preferably, R⁴, R⁵, R⁶ and R⁷ are H. The compound of formula (II) thus has preferably the structure

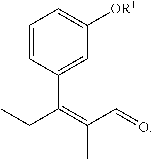

Thus, the present invention also relates to a process, as described above, as well as to an acid salt (T) obtained or obtainable by said process, wherein (a1) comprises providing a compound of the following

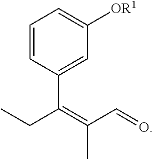

and converting the compound to the compound of formula (I).

### Method 1: Preparation of a Compound of Formula (I)

The present invention is further directed to a method (Method 1) for preparing the compound of formula (I). The compound of formula (I) which has been prepared according to Method 1 can be further reacted to the corresponding acid salt (T), according to a process as disclosed above. In this case, step (a) of the process for preparing acid salt (T) comprises the steps of Method 1 as disclosed below.

Hence Method 1 or step (a) of the process for preparing an acid salt (T) of a compound of formula (I) comprises the steps as disclosed herein under.

The present invention relates to a process for the preparation of a compound of formula (I), and a compound of formula (I) obtained or obtainable by said process of Method 1.

The process of Method 1 and step (a) of the process for preparing an acid salt (T) comprise (a1) providing a compound of the following structure

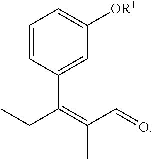

and converting the compound to the compound of formula (I).

As described above with respect to the compound of formula (I), also in the compound of formula (II), residue R¹ is selected from the group consisting of alkyl, aryl, cycloalkyl, heteroalkyl, heteroaryl and heterocycloalkyl. Preferably, R¹ is selected from the group consisting of methyl, ethyl, propyl, isopropyl and benzyl. More preferably, R¹ is methyl.

Thus, the present invention also relates to a process, as described above, wherein R¹ is methyl, more preferably, wherein R¹ is methyl and wherein R⁴, R⁵, R⁶ and R⁷ are H.

As mentioned above, the conversion to compound (I) includes the reaction of the compound of formula (II) with an amine and the reduction of the resulting compound.

The reaction with the amine may be carried out by any method known to those skilled in the art. In this reaction, the carbonyl group of the compound of formula (II) is reacted with the amine HN(R²)(R³) to give an imine

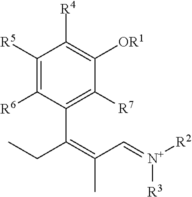

which is then being reduced directly or in a further step to give the corresponding amine. Further, the process comprises the reduction of the double bond shown as “═” in the formulas above.

The reaction with the amine and the reduction of the resulting imine to the corresponding amine may e.g. be carried out in two separate steps or e.g. in one step, in the form of a reductive amination. The double bond may be reduced concurrently with or subsequently to the reduction of the amine. In case, the first step is a reductive amination, the reductive amination is preferably carried out in the presence of a borohydride catalyst and the remaining double bond (shown as “═”) is subsequently reduced with hydrogen and a suitable catalyst, such as a palladium containing catalyst to give the compound of formula (III)

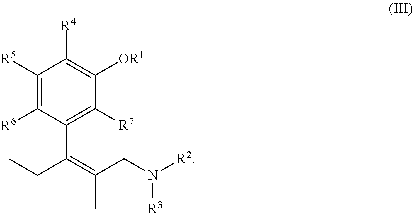

Alternatively, first the imine is formed which is then concurrently with the double bond reduced in one step to directly give the compound of formula (I). In this case, the reduction is e.g. carried out with hydrogen and a suitable catalyst, such as a palladium containing catalyst.

As to the nature of the amine, in this amine HN(R²)(R³), residues R² and R³ are preferably, independently of each other, selected from the group consisting of alkyl, aryl, cycloalkyl, heteroalkyl, heteroaryl and heterocycloalkyl. More preferably, R² and R³, are both alkyl groups, preferably, independently of each other, selected from the group consisting of methyl, ethyl, propyl and isopropyl, more preferably, R² and R³, are methyl, the amine thus having the structure HN(CH₃)₂.

In case the first reaction step is a reductive amination, the process of Method 1 or step (a) of the process for the preparation of the tartaric acid salt (T) disclosed above comprises:
(a1) providing a compound of formula (II)

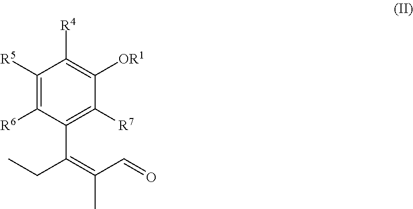

and
(a2) reacting the compound of formula (II) with HN(R²)(R³), in the presence of a suitable reducing agent thereby forming a compound of formula (III)

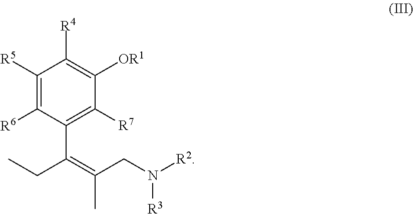

Compound (III) is then optionally isolated and then further reduced to give the compound of formula (I). Alternatively, the reduction of compound (III) is carried out in situ. The further reduction is preferably carried out in the presence of hydrogen employing a suitable catalyst, preferably a palladium comprising catalyst, more preferably Pd/C.

Thus, the present invention also relates to a process (Method 1) for the preparation of a compound of formula (I), as described above or to a process for the preparation of a tartaric acid salt (T) of the compound of formula (I) as described above, wherein the process of Method 1 or step (a) as disclosed above comprises
(a1) providing a compound of formula (II)

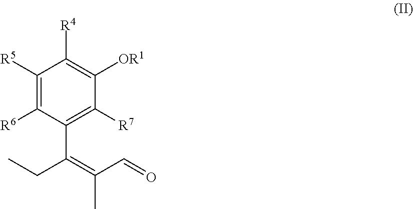

(a2) reacting the compound of formula (II) with HN(R²)(R³), in the presence of a reducing agent, preferably a borohydride, thereby forming a compound of formula (III)

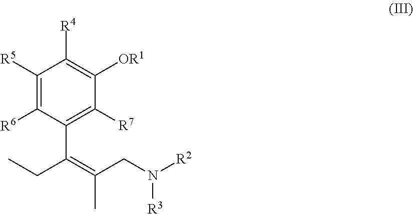

(a3) optionally isolating the compound of formula (III),
(a4) reducing the compound of formula (III) with hydrogen in the presence of a catalyst, preferably Pd/C,
   to give the compound of formula (I).

Hence, the present invention relates to a process for the preparation of a compound of formula (I), and a compound of formula (I), obtained or obtainable by said process, wherein the process comprises
(a1) providing a compound of formula (II)

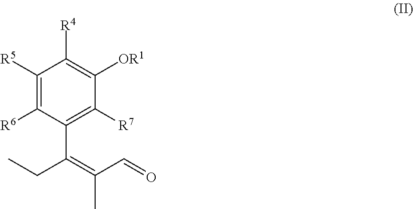

(a2) reacting the compound of formula (II) with HN(R²)(R³), in the presence of a reducing agent, preferably a borohydride, thereby forming a compound of formula (III)

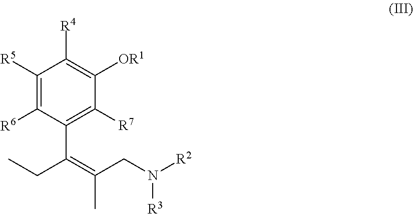

(a3) optionally isolating the compound of formula (III),
(a4) reducing the compound of formula (III) with hydrogen in the presence of a catalyst, preferably Pd/C,
   to give the compound of formula (I).

### Step (a2)

In step (a2), reacting the compound of formula (II) with HN(R²)(R³), in the presence of a reducing agent, preferably a borohydride

As to the solvent used in step (a2), any suitable organic solvent in which the compound of formula (II) is sufficiently soluble may be used. In particular, the solvent is formic acid or acetic acid or a mixtures of both. More preferably, the suitable solvent is acetic acid.

As hydrogen source, preferably hydrogen or formic acid is employed.

The reduction I (a2) is preferably carried out at a temperature of about 0° C. to about 100° C. for about 1 h to about 48 h, such as overnight, in the presence of a suitable reducing agent. Preferably, the reducing agent is a borohydride, such as sodium borohydride, sodium cyanoborohydride, sodium triacetoxy borohydride, organic borane complex compounds such as a 4-(dimethylamino)pyridine borane complex, N-ethyldiisopropylamine borane complex, N-ethylmorpholine borane complex, N-methylmorpholine borane complex, N-phenylmorpholine borane complex, lutidine borane complex, triethylamine borane complex, or trimethylamine borane complex.

Preferably, the reducing agent in this case is selected from the group consisting of sodium borohydride, sodium cyanoborohydride and sodium triacetoxy borohydride, more preferably, the reducing agent is sodium triacetoxy borohydride.

The concentration of these reducing agents used for this reaction of the present invention is preferably in the range of from 0.05 to 0.25 mol/l, more preferably in the range of from 0.01 to 1.0 mol/l, to the volume of the reaction solution.

Generally, when providing the reaction mixture to be reacted in (a2), the sequence of mixing the components of the reaction mixture is not subjected to specific restrictions. Preferably, the compound of formula (II) is first admixed with at least a portion of the solvent, and to the resulting mixture, the amine and acetic acid are added which, for example, can be employed as mixture with at least a portion of the solvent. Preferably, the reductive amination agent as added to the mixture afterwards.

### Step (a3)

In the optional step (a3), the compound of formula (III) is isolated. If such isolation is carried out, this may be carried out by any method known to those skilled in the art. Such isolation may comprise one or more stages wherein preferably at least one stage comprises a purification, such as an extraction and/or a precipitation and/or filtration. Optionally, such working up may comprise at least one stage which comprises a pH adjustment, preferably an adjustment to a pH of at least 8, preferably at least 9, more preferably in the range of from 9 to 11

Adjusting the pH of the reaction mixture to a value of at least 8, preferably at least 9, more preferably from 9 to 11 can be realized, if carried out, according to all conceivable methods. Preferably, an inorganic base, preferably an alkali metal base and/or an alkaline earth metal base, more preferably an alkali metal hydroxide and/or an alkaline earth metal hydroxide, more preferably an alkaline metal hydroxide, more preferably sodium hydroxide is added in a suitable amount. The addition of such a basic compound can be performed at the temperature of the reaction mixture of the reductive amination reaction. Preferably, the reaction mixture obtained from the reductive amination reaction is cooled before the basic compound is added, preferably to a temperature in the range of from 10 to 35° C., more preferably from 20 to 30° C. During adding the basic compound, the mixture can be suitably stirred. The pH is to be understood as the value indicated by a pH sensitive glass electrode without correction.

In case, the compound of formula (III) is precipitated, this precipitation may be carried out by addition of an acid, such as HCl in order to obtain the slat of the compound of formula (III).

Preferably, the compound of formula (III) or the salt thereof is dried in step (III), such as under vacuum, e.g. via lyophilization. The present invention thus also relates to a compound of formula (III)

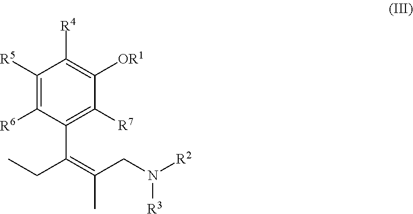

wherein R¹ is selected from the group consisting of alkyl, aryl, cycloalkyl, heteroalkyl, heteroaryl and heterocycloalkyl, R² and R³, are, independently of each other, selected from the group consisting of alkyl, aryl, cycloalkyl, heteroalkyl, heteroaryl and heterocycloalkyl, and wherein R⁴, R⁵, R⁶ and R⁷, are independently of each other, selected from the group consisting of H, alkyl, aryl, cycloalkyl, heteroalkyl, heteroaryl and heterocycloalkyl.

As already outlined above, R⁴, R⁵, R⁶ and R⁷, are preferably, H, the compound thus having the structure

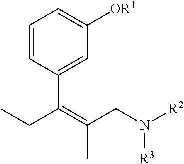

R¹ is preferably, selected from the group consisting of methyl, ethyl, propyl, isopropyl and benzyl, more preferably R¹ is methyl, the compound thus preferably having the structure

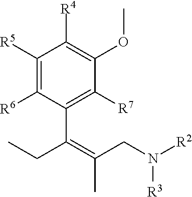

more preferably

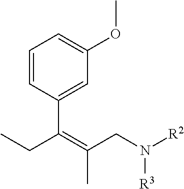

R² and R³, are preferably both alkyl groups, preferably, independently of each other, selected from the group consisting of both methyl, ethyl, propyl and isopropyl, more preferably, R² and R³, are methyl, the compound thus having the structure

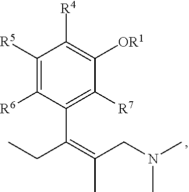

more preferably

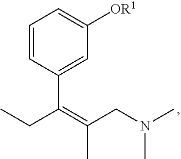

more preferably

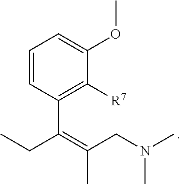

As already mentioned above, the compound of formula (III) has the structure (IIIa) or (IIIb) or consists of a mixture of (IIIa) and (IIIb)

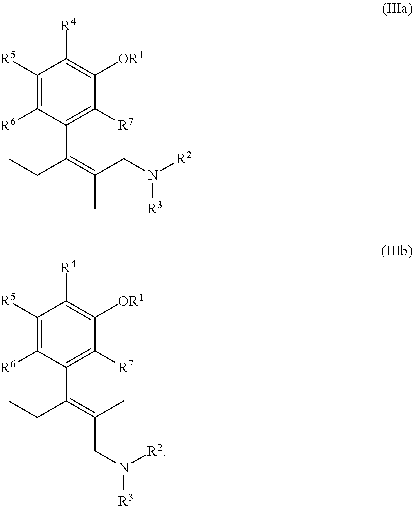

### Step (a4)

The reduction of the double bond in compound (III) is then carried out in a subsequent step. In this case, the reduction of the double bond may be carried out at a temperature of about 0° C. to about 100° C. for about 1 h to about 48 h, such as overnight, in the presence of a suitable catalyst such as Pd/C.

Alternatively, the reaction with the amine may e.g. be carried out in one step to give the imine and the imine as well as the double bond are reduced in a subsequent step to five the compound of formula (I).

Preferably, the reducing agent is a selected from the group consisting of Pd, Pd/C, Pt or Pt/C, most preferably the reducing agent is Pd/C.

The amount of the reducing agent used for this reaction to the total amount of the compound of formula (III) is preferably in the range of from 0.01 to 1, more preferably in the range of from 0.01 to 0.1 mol/l.

As to the solvent used in step (a4), any suitable organic solvent known to those skilled in the art may be used. In particular, the solvent is selected from the group consisting of methanol, ethanol, 2-propanol, propanol, n-butanol, sec-butanol, iso-butanol, and mixtures of two or more thereof. More preferably, the suitable solvent is methanol The reduction is preferably carried out at a pressure in the range of from 1 to 10, more preferably in the range of from 1 to 5, more preferably in the range of from 1 to 3 bar.

After having finished the reduction in (a4) it is conceivable that the reaction mixture obtained may be subjected to a suitable work-up. Such working up may comprise one or more stages wherein preferably at least one stage comprises a purification, such as an extraction and/or a precipitation and/or filtration. Optionally, such working up may comprise at least one stage which comprises a pH adjustment.

Preferably, the compound of formula (I) obtained is purified and dried after step (a4), such as under vacuum, e.g. via lyophilization.

According to a further preferred embodiment of the invention, the imine bond and the double bond are reduced in one step using the same catalyst. Thus, the present invention also relates to a method for the preparation of an acid salt, and the acid salt obtained or obtainable by said method, wherein step (a) of comprises
(a1) providing a compound of formula (II)

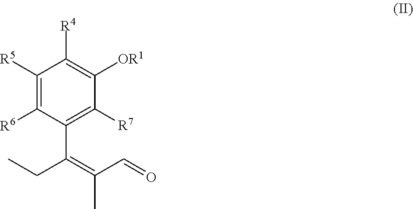

(a2*) reacting the compound of formula (II) with an amine HN(R²)(R³) and reducing the resulting reaction product with hydrogen in the presence of a catalyst, preferably in the presence of Pd/C,
   to give the compound of formula (I).

Further, the present invention also relates to a process for the preparation of a compound of formula (I), and a compound of formula (I) obtained or obtainable by said process, wherein the process comprises
(a1) providing a compound of formula (II)

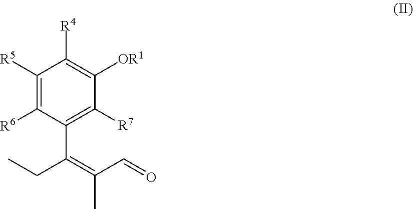

(a2*) reacting the compound of formula (II) with an amine HN(R²)(R³) and reducing the resulting reaction product with hydrogen in the presence of a catalyst, preferably in the presence of Pd/C,
   to give the compound of formula (I).
   Step (a2*)

In step (a2*), the compound of formula (II) is reacted with HN(R²)(R³) and the resulting reaction product, preferably the compound of formula (III)

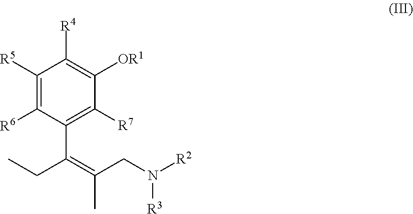

is then reduced with hydrogen in the presence of a catalyst. Step (a2*) may comprise at least one working-up step between the reaction with HN(R²)(R³) and the subsequent reduction step. Such working-up steps include purification steps, removal of solvent, isolation and optionally drying of the compound of formula (III) and option redissolving of the compound of formula (III). Preferably, the reaction with HN(R²)(R³) and the subsequent reduction step are carried out in situ, i.e. without an intermediate working up step.

As to the solvent used in step (a2*) for the reaction with HN(R²)(R³) this solvent is not particular restricted. Preferably, the solvent is selected from the group consisting of methanol ethanol, 2-propanol, propanol, n-butanol, sec-butanol, iso-butanol and mixtures of two or more thereof. More preferably, the suitable solvent is methanol.

As to the solvent used in step (a2*) for reduction step, this solvent is not particular restricted. Preferably, the solvent is selected from the group consisting of methanol ethanol, 2-propanol, propanol, n-butanol, sec-butanol, iso-butanol and mixtures of two or more thereof. More preferably, the suitable solvent is methanol. Most preferably, the same solvent is used during the reaction with the amine HN(R²)(R³) and the reduction.

The reduction I (a2) is preferably carried out at a temperature of about 0° C. to about 100° C. for about 1 h to about 240 h, such 24 to 200 h, preferably 100 to 150 h, in the presence of a suitable catalyst and a hydrogen source, preferably hydrogen. Preferably, the catalyst is a palladium comprising catalyst, more preferably, Pd/C.

Generally, when providing the reaction mixture to be reacted in (a2*), the sequence of mixing the components of the reaction mixture is not subjected to specific restrictions. Preferably, the compound of formula (II) is first admixed with at least a portion of the solvent, and to the resulting mixture, the amine and the catalyst are added. Afterwards, the reduction is preferably carried out in an atmosphere comprising hydrogen, preferably comprising at least 80 Vol % of hydrogen. The amount of the reducing agent used for this reaction to the total amount of the compound of formula (III) is preferably in the range of from 0.01 to 1, more preferably in the range of from 0.01 to 0.1 mol/l.

The reduction is preferably carried out at a pressure in the range of from 1 to 10, more preferably in the range of from 1 to 5, more preferably in the range of from 1 to 3 bar.

After having finished the reduction in (a2*) it is conceivable that the reaction mixture obtained may be subjected to a suitable work-up. Such working up may comprise one or more stages wherein preferably at least one stage comprises a purification, such as an extraction and/or a precipitation and/or filtration. Optionally, such working up may comprise at least one stage which comprises a pH adjustment.

Preferably, the compound of formula (I) obtained is purified and dried after step (a2*), such as under vacuum, e.g. via lyophilization.

### Step (a1)

In step (a1) of the methods described hereinunder and above, a compound of formula (II) is provided. This provision is not particular restricted and includes e.g. any possible synthesis of this compound. Preferably step (a1) comprises
(a1.1) providing a compound of formula (IV)

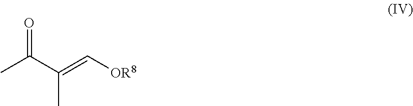

wherein R⁸ is selected from the group consisting of methyl, ethyl, isopropyl, ter.-butyl and —CH(isopropyl)₂,
(a1.2) reacting the compound of formula (IV) with an organometallic compound of formula (V),

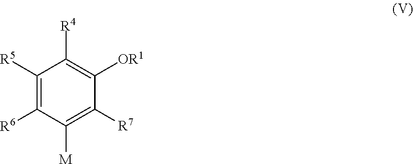

wherein M is selected from the group consisting of Li, MgBr, CuMgBr, CuLi and

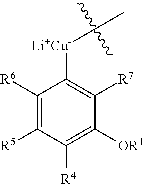

to give the compound of formula (II)

Thus, the present invention also relates to a process, as described above, and to a tartaric acid salt (T) as described above, wherein step (a) comprises
(a1) providing a compound of formula (II)

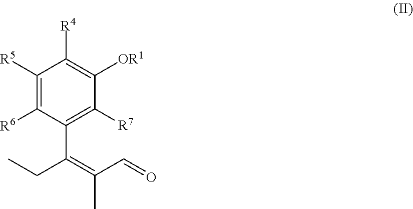

wherein the provision comprises
(a1.1) providing a compound of formula (IV)

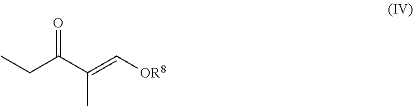

wherein R⁸ is selected from the group consisting of methyl, ethyl, isopropyl, ter.-butyl and —CH(isopropyl)₂,
(a1.2) reacting the compound of formula (IV) with an organometallic compound of formula (V),

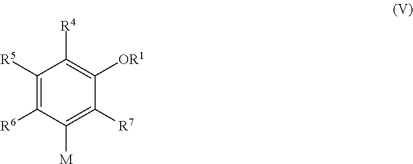

wherein M is selected from the group consisting of Li, MgBr, CuMgBr, CuLi and

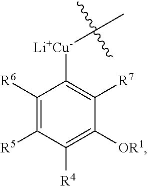

to give the compound of formula (II)
(a2) reacting the compound of formula (II) with HN(R²)(R³) in the presence of a reducing agent, preferably a borohydride, thereby forming a compound of formula (III)

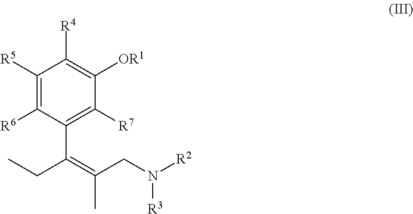

(a3) optionally isolating the compound of formula (III),
(a4) reducing the compound of formula (III) with hydrogen in the presence of a catalyst, preferably Pd/C,
   to give the compound of formula (I).

Further, the present invention relates to a process for the preparation of a compound of formula (I), and a compound of formula (I), obtained or obtainable by said process, wherein the process comprises
(a1) providing a compound of formula (II)

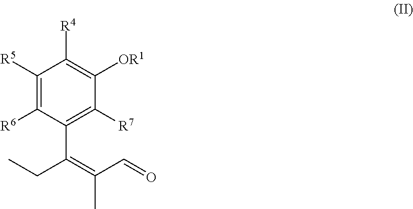

wherein the provision comprises
(a1.1) providing a compound of formula (IV)

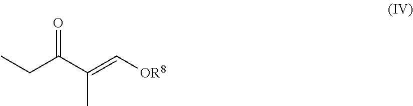

wherein R⁸ is selected from the group consisting of methyl, ethyl, isopropyl, ter.-butyl and —CH(isopropyl)₂,
(a1.2) reacting the compound of formula (IV) with an organometallic compound of formula (V),

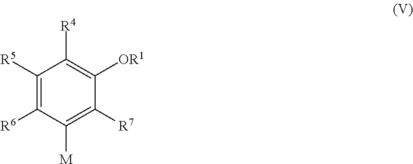

wherein M is selected from the group consisting of Li, MgBr, CuMgBr, CuLi and

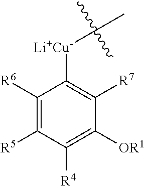

to give the compound of formula (II)
(a2) reacting the compound of formula (II) with HN(R²)(R³) in the presence of a reducing agent, preferably a borohydride, thereby forming a compound of formula (III)

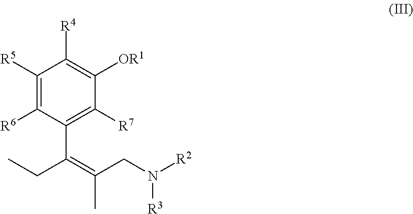

(a3) optionally isolating the compound of formula (III),
(a4) reducing the compound of formula (III) with hydrogen in the presence of a catalyst, preferably Pd/C,
   to give the compound of formula (I).

Further, the present invention also relates to a method for the preparation of a tartaric acid salt, and the acid salt obtained or obtainable by said method, wherein step (a) of comprises
(a1) providing a compound of formula (II)

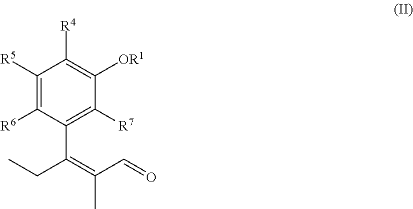

wherein the provision comprises
(a1.1) providing a compound of formula (IV)

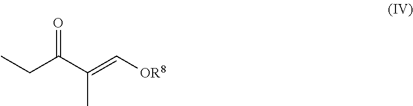

wherein R⁸ is selected from the group consisting of methyl, ethyl, isopropyl, ter.-butyl and —CH(isopropyl)₂,
(a1.2) reacting the compound of formula (IV) with an organometallic compound of formula (V),

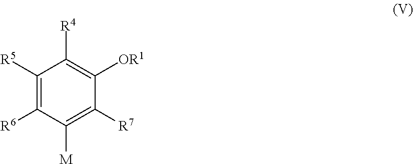

wherein M is selected from the group consisting of Li, MgBr, CuMgBr, CuLi and

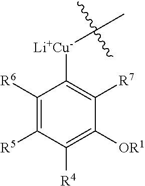

to give the compound of formula (II),
(a2*) reacting the compound of formula (II) with an amine HN(R²)(R³) and reducing the resulting reaction product with hydrogen in the presence of a catalyst, preferably in the presence of Pd/C,
   to give the compound of formula (I).

Further, the present invention also relates to a process for the preparation of a compound of formula (I), and a compound of formula (I) obtained or obtainable by said process, wherein the process comprises
(a1) providing a compound of formula (II)

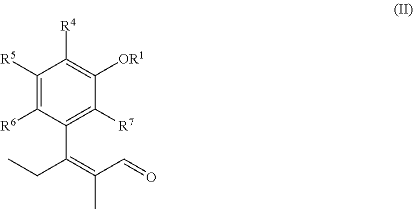

wherein the provision comprises
(a1.1) providing a compound of formula (IV)

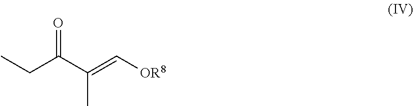

wherein R⁸ is selected from the group consisting of methyl, ethyl, isopropyl, ter.-butyl and —CH(isopropyl)₂,
(a1.2) reacting the compound of formula (IV) with an organometallic compound of formula (V),

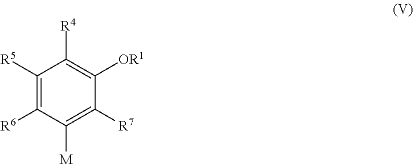

wherein M is selected from the group consisting of Li, MgBr, CuMgBr, CuLi and

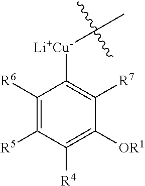

to give the compound of formula (II)
(a2*) reacting the compound of formula (II) with an amine HN(R²)(R³) and reducing the resulting reaction product with hydrogen in the presence of a catalyst, preferably in the presence of Pd/C,
   to give the compound of formula (I).

Preferably wherein R⁸ in compound is —CH(isopropyl)₂, the compound thus having the structure

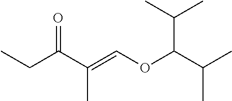

Thus, the present invention also relates to a compound of formula (IV), wherein R⁸ is —CH(isopropyl)₂.

### Step (a1.1)

In step (a.1.1) a compound of formula (IV) is provided. This is preferably carried out by a method comprising
(a1.1.1) providing a compound of formula (VII)

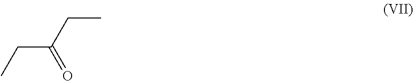

(a1.1.2) reacting the compound of formula (VII) in the presence of sodium hydride with HC(O)OR⁹ to give a compound of formula (VIII) or a sodium salt thereof

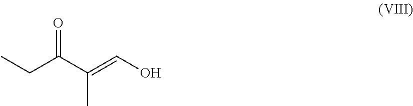

(a1.1.3) reacting the compound of formula (VIII) with R⁸—OH, wherein R⁸ is selected from the group consisting of methyl, ethyl, isopropyl, ter.-butyl and —CH(isopropyl)₂
   to give the compound of formula (IV).

### Step (a.1.1.2)

The reaction in (a1.1.2) is preferably carried out in an organic solvent, preferably a solvent selected from the group consisting of toluene, xylol, n-heptane and a mixture of two or more thereof. Preferably, the solvent is selected from the group consisting of toluene, xylol, n-heptane and a mixture of two or more thereof. Preferably, the solvent contains less than 10 ppm of water. More preferably, the reaction is carried out in an anhydrous atmosphere, more preferably in an inert atmosphere such as under argon or under nitrogen.

Generally, when providing the reaction mixture to be reacted in (a1.1.2), the sequence of mixing the components of the reaction mixture is not subjected to specific restrictions. However, preferably, first the sodium hydride is suspended in the solvent and at least one alcohol, preferably methanol is added. Subsequently, preferably, the compound of formula (VII) and HC(O)OR⁹ admixed with at least a portion of the solvent are added. Preferably, the addition is carried out drop-wise.

The reaction is preferably carried out at a temperature of at most 15° C., more preferably at most 10° C., preferably at a temperature in the range of from −10 to 10° C., more preferably in the range of from 0 to 10° C.

The addition of the compound of formula (VII) and HC(O)OR⁹ is preferably carried out in such manner that the temperature does not rise above 15° C., more preferably 10° C.

The reaction mixture is preferably allowed to react, preferably while stirring, for about 10 min to about 48 h, such as overnight

Optionally during the reaction a further solvent may be added, such as e.g. diethylether.

The molar ratio of sodium hydride to the compound of formula (VII) of is preferably in the range of from 1.0 to 1.2, more preferably in the range of 1.0 to 1.1.

Residue R⁹ in HC(O)OR⁹ is preferably a substituted or unsubstituted alkyl or cycloalkyl group, preferably, a methyl, ethyl, n-propyl, isopropyl, n-butyl, isobutyl, tert-butyl, n-pentyl, cyclopropyl, cyclobutyl, cyclopentyl, cyclohexyl or cycloheptyl group, more preferably a methyl, ethyl, n-propyl, isopropyl, n-butyl, isobutyl, tert-butyl or n-pentyl group, more preferably, a methyl, or ethyl group, more preferably methyl.

The compound of formula (VIII) or the sodium salt thereof is preferably isolated. If such isolation is carried out, this may be carried out by any method known to those skilled in the art. Such isolation may comprise one or more stages wherein preferably at least one stage comprises a purification, such as an extraction and/or a precipitation and/or filtration. In case, the compound of formula (VIII) is precipitated, this precipitation may be carried out by addition of a further solvent such as diethylether, as mentioned above. Preferably, the compound of formula (VIII) or the salt thereof is dried in step (a1.1.3), such as under vacuum, e.g. via lyophilization.

Preferably, in step (a1.1.2) the sodium salt of the compound of formula (VIII) is obtained.

### Step (a1.1.3)

In step (a1.1.3) the compound of formula (VIII) or the salt thereof is reacted with R⁸—OH wherein R⁸ is selected from the group consisting of methyl, ethyl, isopropyl, ter.-butyl and —CH(isopropyl)₂.

Preferably, the reaction in (a1.1.3) is carried out in R⁸—OH as solvent.

In (a1.1.3) the compound of formula (IV) is formed. Preferably, the reaction in (a1.1.3) is carried out in the presence of at least one acid, such as citric acid or p-toluene sulfonic acid, preferably of p-toluene sulfonic acid.

Generally, when providing the reaction mixture to be reacted in (a1.1.3), the sequence of mixing the components of the reaction mixture is not subjected to specific restrictions. However, preferably, first the compound of formula (VIII) or the salt thereof is admixed with R⁸—OH, and optionally a further organic solvent, such as DMSO or acetone, preferably with R⁸—OH as solvent and, if present, the at least one acid, preferably of p-toluene sulfonic acid is added.

The reaction is preferably carried out at a temperature of at most 15° C., more preferably at most 10° C., preferably at a temperature in the range of from −10 to 10° C., more preferably in the range of from 0 to 10° C.

The addition of the compound of formula (VII) and HC(O)OR⁹ is preferably carried out at a temperature in the range of from 0 to 30° C., more preferably in the range of from 0 to 20° C., more preferably in the range of from 0 to 10° C.

The reaction mixture is preferably allowed to react, preferably while stirring, for about 10 min to about 48 h, preferably for a time in the range of from 1 h to 24 h, more preferably of from 6 to 14 h, such as overnight.

The resulting reaction product is preferably subjected to at least one working-up step, such as purification by chromatography, distillation or the like.

Preferably, the solvent is afterwards at least partially removed, such as preferably by distillation. Preferably essentially all of the solvent is removed. The term “essentially all” in this context is denoted to mean that at least 99% by weight, more preferably at least 99.9% by weight of the solvent is removed.

Most preferably, R⁸ is —CH(isopropyl)₂.

Thus, the present invention also relates to a compound of formula (IV)

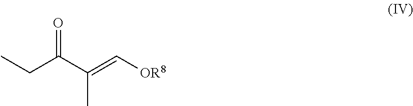

wherein R⁸ is —CH(isopropyl)₂.

### Step (a1.2)

As outlined above, in step (a1.2), the compound of formula (IV) is preferably reacted with an organometallic compound (V) to give the compound of formula (II) the compound of formula (V) having the structure,

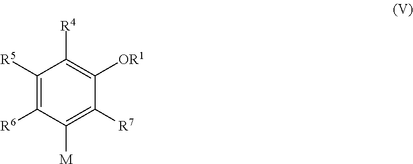

wherein M is selected from the group consisting of Li, MgBr, CuMgBr, CuLi, and

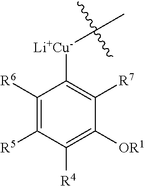

and wherein R⁴, R⁵, R⁶ and R⁷, are independently of each other, selected from the group consisting of H, alkyl, aryl, cycloalkyl, heteroalkyl, heteroaryl and heterocycloalkyl. Preferably, R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl cycloheptyl. More preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl. More preferably, R⁴, R⁵, R⁶ and R⁷ are H.

Thus, in step (a1.2), the compound of formula (IV) is preferably reacted with an organometallic compound (V) having the structure

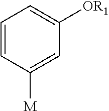

As described above, residue R¹ is selected from the group consisting of alkyl, aryl, cycloalkyl, heteroalkyl, heteroaryl and heterocycloalkyl. Preferably, R¹ is selected from the group consisting of methyl, ethyl, propyl, isopropyl and benzyl. More preferably, R¹ is methyl. Thus, R in (R)ₙM is preferably

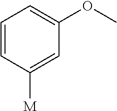

As mentioned above, M is selected from the group consisting of Li, MgBr, CuMgBr, CuLi, and

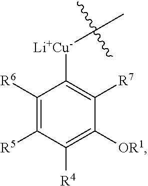

preferably Li, MgBr, CuMgBr, and

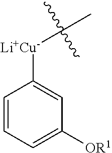

more preferably of Li, MgBr, and

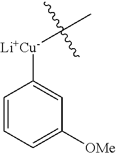

Preferably M is Li. In this case, the organometallic compound (V) is preferably prepared by reacting a compound of formula (VI)

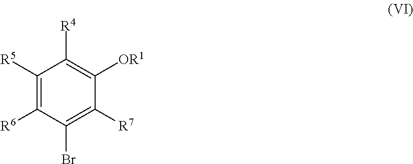

wherein R⁴, R⁵, R⁶ and R⁷, are independently of each other, selected from the group consisting of H, alkyl, aryl, cycloalkyl, heteroalkyl, heteroaryl and heterocycloalkyl, preferably, wherein R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl, more preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl, and wherein most preferably, R⁴, R⁵, R⁶ and R⁷ are H, and wherein R¹ is selected from the group consisting of alkyl, aryl, cycloalkyl, heteroalkyl, heteroaryl and heterocycloalkyl, preferably from the group consisting of methyl, ethyl, propyl, isopropyl and benzyl, more preferably, wherein R¹ is methyl, with a buthyllithium reagent, in particular nBuLi.

This preparation is preferably carried out at a temperature of less than −70° C., more preferably at a temperature in the range of from −70° C. to −80° C., such as in particular around −78° C. As to the solvent used, preferably an anhydrous organic solvent or a mixture of anhydrous solvents is employed. Preferably, the reaction is carried out in a solvent selected from the group consisting of, preferably anhydrous, THF, hexane, toluene, MeTHF, Et₂O, ⁱPr₂O, MTBE (Methyl-tert-butylether), dimethoxyethane, and mixtures of two or more thereof. The reaction mixture comprising the compound of formula (VI), the Buthyllithium reagent and the at least one solvent is preferably allowed to react for a time in the range of from 10 min to 6 h, preferably 15 min to 1 h, more preferably 20 min to 40 min.

Most preferably, the organometallic compound (V) is

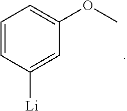

The reaction between the compound of formula (V) and the compound of formula (IV) in (a1.2) is preferably carried out in a solvent selected from the group consisting of glyme, diglyme and BF₃-etherate, preferably in glyme, or in a mixture of solvents comprising a solvent selected from the group consisting of glyme, diglyme and BF₃-etherate.

Preferably, the preparation of the organometallic compound (V) and the subsequent reaction with the compound of formula (IV) is carried out in one pot without any previous isolation of the compound of formula (V). Thus, in this case the reaction is preferably carried out in a solvent system comprising at least one solvent selected from the group consisting of THF, hexane, toluene, MeTHF, Et₂O, ⁱPr₂O, MTBE (Methyl-tert-butylether), dimethoxyethane and mixtures of two or more thereof, and at least one solvent selected from the group consisting of glyme, diglyme and BF₃-etherate

The reaction is preferably carried out at a temperature of at most −60° C., more preferably at most −65° C., preferably at a temperature in the range of from −65 to −80° C., more preferably in the range of from −70 to −80° C.

The reaction mixture is preferably allowed to react, preferably while stirring, for about 10 min to about 48 h, preferably for a time in the range of from 1 h to 24 h, more preferably of from 2 to 8 h.

The resulting crude reaction product is preferably subjected to at least one working-up step, such as purification by chromatography, distillation or the like.

### Method 2: Preparation of a Compound of Formula (I)

The present invention is further directed to a method (Method 2) for preparing the compound of formula (I). The compound of formula (I) which has been prepared according to Method 2 can be further reacted to the corresponding acid salt (T), according to a process as disclosed above. In this case, step (a) of the process for preparing acid salt (T) comprises the steps of Method 2 as disclosed below.

Hence, Method 2 or step (a) of the process for preparing an acid salt (T) of a compound of formula (I) comprises the steps
(aa) providing a compound of formula (IX)

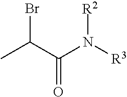

wherein R² and R³, are, independently of each other, selected from the group consisting of alkyl, aryl, cycloalkyl, heteroalkyl, heteroaryl and heterocycloalkyl, more preferably, R² and R³, are both alkyl groups, preferably, independently of each other, selected from the group consisting of methyl, ethyl, propyl and isopropyl, more preferably, R² and R³, are methyl,
(ab) providing a compound of formula (X)

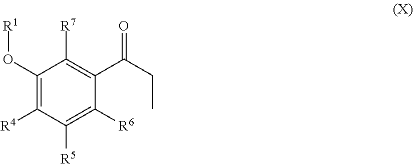

Wherein R⁴, R⁵, R⁶ and R⁷, are independently of each other, selected from the group consisting of H, alkyl, aryl, cycloalkyl, heteroalkyl, heteroaryl and heterocycloalkyl, preferably, wherein R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl and cyclohexyl cycloheptyl, more preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl, and wherein most preferably, R⁴, R⁵, R⁶ and R⁷ are H, and wherein R¹ is selected from the group consisting of alkyl, aryl, cycloalkyl, heteroalkyl, heteroaryl and heterocycloalkyl, preferably from the group consisting of methyl, ethyl, propyl, isopropyl and benzyl, more preferably, wherein R¹ is methyl,
(ac) reacting the compound of formula (IX) with the compound of formula (X) in the presence of Zn to give a compound of formula (XI)

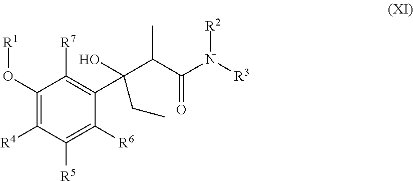

(ad) dehydrating the compound of formula (XI) to give a compound of formula (XIIa)

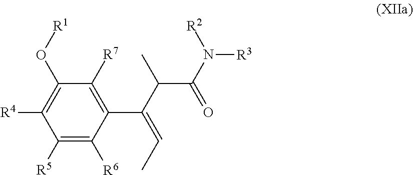

and/or a compound of formula (XIIb)

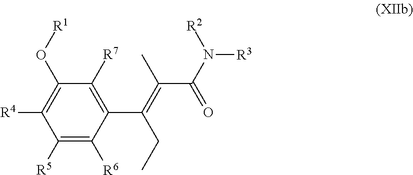

(ae) reducing the compound of formula (XIIa) and/or (XIIb),
   to give the compound of formula (I).

Thus, the present invention also relates to a process (Method 2) for the preparation of a compound of formula (I), as described above or to a process for the preparation of a tartaric acid salt (T) of the compound of formula (I) as described above, wherein the process of Method 2 or step (a) of the process as disclosed above comprises
(a1) providing a compound of formula (II)

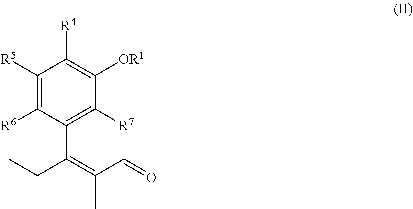

wherein the provision comprises
(aa) providing a compound of formula (IX)

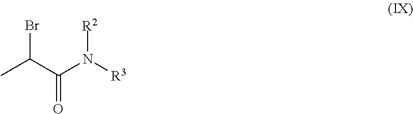

wherein R² and R³, are, independently of each other, selected from the group consisting of alkyl, aryl, cycloalkyl, heteroalkyl, heteroaryl and heterocycloalkyl, more preferably, R² and R³, are both alkyl groups, preferably, independently of each other, selected from the group consisting of methyl, ethyl, propyl and isopropyl, more preferably, R² and R³, are methyl,
(ab) providing a compound of formula (X)

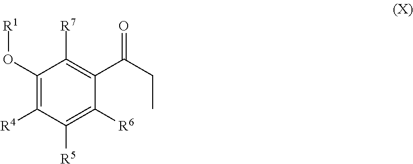

wherein R⁴, R⁵, R⁶ and R⁷, are independently of each other, selected from the group consisting of H, alkyl, aryl, cycloalkyl, heteroalkyl, heteroaryl and heterocycloalkyl, preferably, wherein R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl, more preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl, and wherein most preferably, R⁴, R⁵, R⁶ and R⁷ are H, and wherein R¹ is selected from the group consisting of alkyl, aryl, cycloalkyl, heteroalkyl, heteroaryl and heterocycloalkyl, preferably from the group consisting of methyl, ethyl, propyl, isopropyl and benzyl, more preferably, wherein R¹ is methyl,
(ac) reacting the compound of formula (IX) with the compound of formula (X) in the presence of Zn to give a compound of formula (XI)

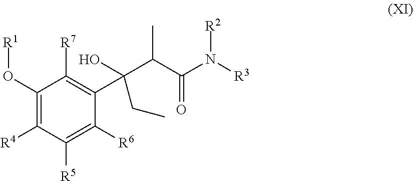

(ad) dehydrating the compound of formula (XI) to give a compound of formula (XIIa)

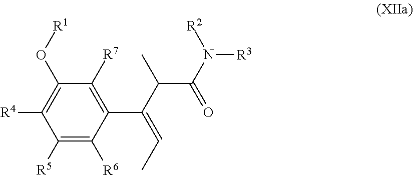

and/or a compound of formula (XIIb)

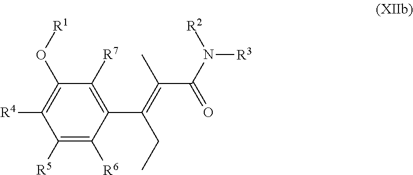

(ae) reducing the compound of formula (XIIa) and/or (XIIb),
to give the compound of formula (I).

Further, the present invention relates to a process (Method 2) for the preparation of a compound of formula (I), and a compound of formula (I), obtained or obtainable by said process, wherein the process comprises
(aa) providing a compound of formula (IX)

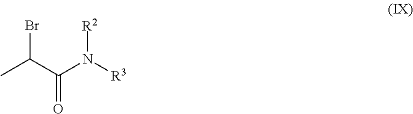

wherein R² and R³, are, independently of each other, selected from the group consisting of alkyl, aryl, cycloalkyl, heteroalkyl, heteroaryl and heterocycloalkyl, more preferably, R² and R³, are both alkyl groups, preferably, independently of each other, selected from the group consisting of methyl, ethyl, propyl and isopropyl, more preferably, R² and R³, are methyl,
(ab) providing a compound of formula (X)

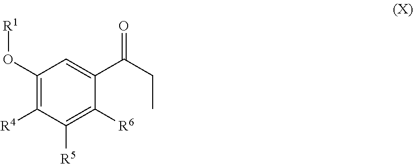

wherein R⁴, R⁵, R⁶ and R⁷, are independently of each other, selected from the group consisting of H, alkyl, aryl, cycloalkyl, heteroalkyl, heteroaryl and heterocycloalkyl, preferably, wherein R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl, more preferably, R⁴, R⁵, R⁶ and R⁷, are independently of each other, H or alkyl, more preferably, independently of each other, selected from the group consisting of H, methyl, ethyl, propyl, butyl, sec-butyl and tert-butyl, and wherein most preferably, R⁴, R⁵, R⁶ and R⁷ are H, and wherein R¹ is selected from the group consisting of alkyl, aryl, cycloalkyl, heteroalkyl, heteroaryl and heterocycloalkyl, preferably from the group consisting of methyl, ethyl, propyl, isopropyl and benzyl, more preferably, wherein R¹ is methyl,
(ac) reacting the compound of formula (IX) with the compound of formula (X) in the presence of Zn to give a compound of formula (XI)

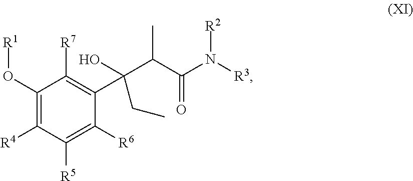

(ad) dehydrating the compound of formula (XI) to give a compound of formula (XIIa)

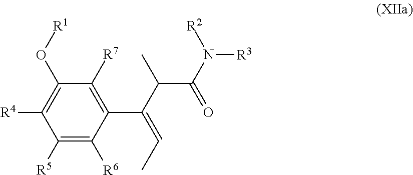

and/or a compound of formula (XIIb)

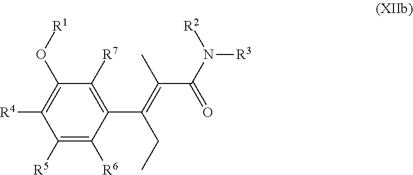

(ae) reducing the compound of formula (XIIa) and/or (XIIb),
to give the compound of formula (I).

### Step (aa)

The way of providing a compound of formula (XIII) is not critical. Preferably, the compound of formula (IX) in (aa) is provided by reacting a compound of formula (XIII)

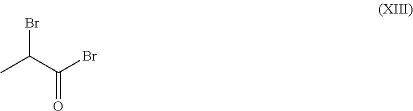

with HN(R²)(R³).

The reaction is preferably carried out at a temperature in the range of from 0 to 50° C., more preferably in the range of from 0 to 25° C.

The reaction mixture is preferably allowed to react, preferably while stirring, for about 10 min to about 12 h, preferably for a time in the range of from 1 h to 4 h, more preferably of from 30 min to 2 h.

The solvent is preferably dichloromethane or chloroform or a mixture thereof.

Preferably, HN(R²)(R³) is HN(CH₃)₂.

The resulting product is preferably subjected to at least one working-up step, such as purification by chromatography, distillation or the like.

### Step (ab)

The compound of formula (X) is provided by reacting

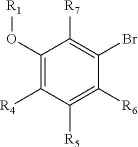

wherein R⁴, R⁵, R⁶ and R⁷, are independently of each other, selected from the group consisting of H, alkyl, aryl, cycloalkyl, heteroalkyl, heteroaryl and heterocycloalkyl, preferably, wherein R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl, more preferably, wherein R⁴, R⁵, R⁶ and R⁷, are independently of each other, H or alkyl, more preferably, independently of each other, selected from the group consisting of H, methyl, ethyl, propyl, butyl, sec-butyl and tert-butylmore, with propanoic anhydride.

Preferably, 1-bromo-3-methoxybenzene is reacted with propanoic anhydride. The reaction is preferably carried out via a Grignard reaction. Suitable conditions for such reaction are known to those skilled in the art.

The resulting product is preferably subjected to at least one working-up step, such as purification by chromatography, distillation or the like.

### Step (ac)

In step (ac), the compound of formula (IX) is reacted with the compound of formula (X) in the presence of Zn to give a compound of formula (XI)

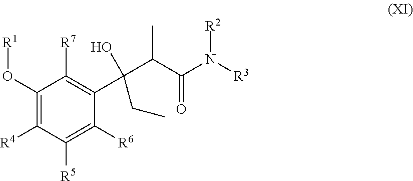

The reaction is preferably carried out at a temperature in the range of from 0 to 100° C., more preferably in the range of from 25 to 90° C., more preferably in the range of from 50 to 75° C.

The reaction mixture is preferably allowed to react, preferably while stirring, for about 1 min to about 24 h, preferably for a time in the range of from 8 to 16 h.

The solvent is preferably THF or Me-THF.

The resulting product is preferably subjected to at least one working-up step, such as purification by chromatography, distillation or the like.

### Step (ad)

The reaction in (ad) is preferably carried out by reacting the compound of formula (XI) with a dehydrating agent. The term “dehydrating agent” refers to any agent capable of dehydrating the compound of formula (XI) to give a compound of formula (XIIa), i.e. to remove water. Such agents include, but are not limited to, acids, such as sulphuric acid, sulfonyl chlorides such as thionyl chloride, methanesulfonyl chloride, ethanesulfonyl chloride, and the like; acid chlorides such as acetyl chloride, benzoyl chloride, and the like; and phosphines such as triphenyl phosphine, trimethylphosphine and the like. The dehydrating agent is preferably sulfuric acid.

The reaction may be carried out in any suitable solvent known to those skilled in the art. Preferably, the reaction in (ad) is carried out in an organic solvent, more preferably in a solvent selected from the group consisting of toluene, Cl-benzene, xylene, nitrobenzene and mixtures of two or more thereof, more preferably in toluene.

Preferably, step (ad) is carried out at a temperature in the range of from 0° C. to 120° C. During the reaction, the temperature may be varied or held essentially constant.

Preferably, in step (ad) water is removed and collected in a trap. Thus, the compound of formula (XI) is preferably allowed to react with the dehydrating agent for a time until no additional water is condensing in the trap.

It is to be understood that in step (ad), either a compound of formula (XIIa) or a compound of formula (XIIb) or a mixture of both compounds is formed. Preferably, thus either the compound of formula (XIIa) or the compound of formula (XIIb) or the mixture of both compounds is employed in the subsequent step (ae).

### Step (ae)

In step (ae), the compound of formula (XIIa) and/or (XIIb), is reduced to give the compound of formula (I). Such reduction may be carried out by any method known to those skilled in the art.

Preferably step (ae) comprises
(ae1) reducing the compound of formula (XIIa) and/or (XIIb) to give a compound of formula (XIIa1)

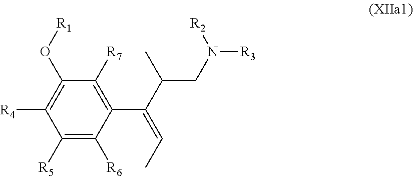

and/or a compound of formula (XIIb1)

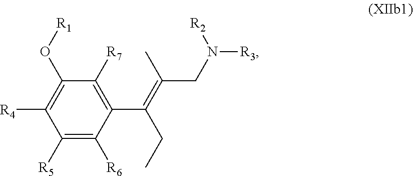

(ae2) reducing the compound of formula (XIIa1) and/or (XIIb1)
   to give the compound of formula (I).

It is to be understood,
that in case in step (ad) a compound of formula (XIIa) is formed, in step (ae1) a compound of formula (XIIa1) is formed, and
that in case in step (ad) a compound of formula (XIIb) is formed, in step (ae1) a compound of formula (XIIb1) is formed, and
that in case in step (ad) a mixture of both compounds is formed, in step (ae1) also a mixture of both compounds is formed.

Preferably, thus either the compound of formula (XIIa1) or the compound of formula (XIIb1) or the mixture of both compounds is employed in the subsequent step (ae2).

Step (ae1)

The reduction of the compound of formula (XIIa) and/or (XIIb) to give a compound of formula (XIIa1) and/or (XIIb1) may be carried out by any method known to those skilled in the art.

Preferably the reduction is carried out by reaction with a reducing agent selected from the group consisting of NaBH₄, NaCNBH₃, NaBH(OAc)₃, LiAlH₄, LiBH₄ and H₂ in the presence of transition metals, wherein the transition metal is preferably selected from the group consisting of IR, Pt, Fe, Rh, Pd, Re, Ru, Ni and Co. More preferably, the reducing agent is selected from the group consisting of NaBH₄, NaCNBH₃, NaBH(OAc)₃, LiAlH₄ and LiBH₄, more preferably the reducing agent is NaBH₄, NaCNBH₃ or NaBH(OAc)₃, more preferably LiAlH₄.

The reaction may be carried out in any suitable solvent known to those skilled in the art. Preferably, the reaction in (ae1) is carried out in an organic solvent, more preferably in a solvent selected from the group consisting of methanol, ethanol, nPrOH (n-propanol), i-PrOH (isopropanol), THF (tetrahydrofuran), 2-MeTHF (2-methyl-tehterhydrofuran), MTBE (Methyl-tert-butylether), DIPET (diisiopropylethylether), toluene, acetonitrile, CH₂Cl₂ and mixtures of two or more thereof.

Preferably, step (ae1) is carried out at a temperature in the range of from −20 to 80° C., more preferably in the range of from 0 to 50, more preferably in the range of from 20 to 30° C. During the reaction, the temperature may be varied or held essentially constant.

The resulting product may be subjected to at least one working-up step, such as isolation of the product by chromatography, distillation or the like. Preferably, the compound(s) obtained in step (ae1) is/are directly employed in step (ae2) without any isolation step.

Step (ae2)

In step (ae2), compound (XIIa1) and/or compound (XIIb1) is/are further reduced to give compound (I).

Preferably this reduction is carried out by reaction with a reducing agent, in particular H2 H₂ in the presence of metal catalyst, preferably a transition metal catalyst, wherein the transition metal is in particular selected from the group consisting of Pt, AU, or Pd. More preferably the reducing agent is H2 in the presence of a Pd catalyst, in particular Pd/C or Palldium(II)chloride.

The reaction may be carried out in any suitable solvent known to those skilled in the art. Preferably, the reaction in (ae1) is carried out in a solvent selected from the group consisting of H₂O, methanol, ethanol, nPrOH (n-propanol), i-PrOH (isopropanol), THF (tetrahydrofuran), 2-MeTHF (2-methyl-tehterhydrofuran), MTBE (Methyl-tert-butylether), DIPET (diisiopropylethylether), toluene, acetonitrile, CH₂Cl₂ and mixtures of two or more thereof.

Preferably, H₂O is employed as solvent.

Optionally, at least one acid may be added, such as HCl. Preferably, the reaction is carried out in a mixture comprising H2O and HCl.

Preferably, step (ae1) is carried out at a temperature in the range of from −20 to 80° C., more preferably in the range of from 0 to 50, more preferably in the range of from 20 to 40° C. During the reaction, the temperature may be varied or held essentially constant.

The reaction is preferably carried out at a hydrogen pressure in the range of from 3 to 10 bar, more preferably, 4 to 7 bar. During the reaction, the pressure may be varied or held essentially constant.

The resulting product is preferably subjected to at least one working-up step, such as purification by chromatography, distillation or the like.

### Compound of Formula (XIV)

Further, the present invention relates to a process for the preparation of a compound of formula (XIV),

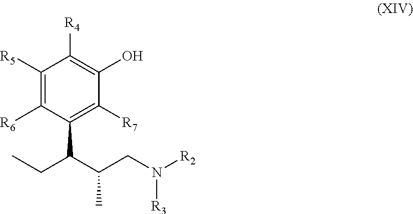

or a pharmaceutically acceptable salt or solvate thereof, and the compound obtained or obtainable by said process, wherein R⁴, R⁵, R⁶ and R⁷, are independently of each other, selected from the group consisting of H, alkyl, aryl, cycloalkyl, heteroalkyl, heteroaryl and heterocycloalkyl. Preferably, R⁴, R⁵, R⁶ and R⁷, are independently of each other, selected from the group consisting of H, methyl, ethyl, propyl, butyl, sec-butyl, tert-butyl, phenyl, cyclopropyl, cyclobutyl, cyclopentyl, cyclohexyl and cycloheptyl and wherein R² and R³, are, independently of each other, selected from the group consisting of alkyl, aryl, cycloalkyl, heteroalkyl, heteroaryl and heterocycloalkyl. This process preferably comprises
(i) providing an acid salt, preferably a tartaric acid salt, (T) of a compound of formula (I)

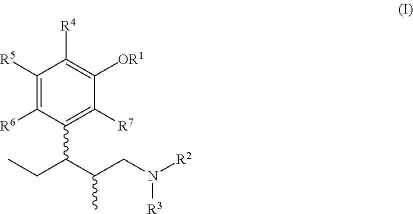

(ii) transforming the group —OR¹ to OH,
(iii) optionally purifying the compound obtained in (ii)
   to give the compound of formula (XIV)
(iv) optionally preparing pharmaceutically acceptable salt or solvate of compound (XIV) of (ii) or (iii)

### Step (i)

The provision in step (i) is carried out by the methods described hereinunder and above, preferably by the method comprising the steps (a) to (c) described above.

Thus, the present invention relates to a process for the preparation of a compound of formula (XIV),

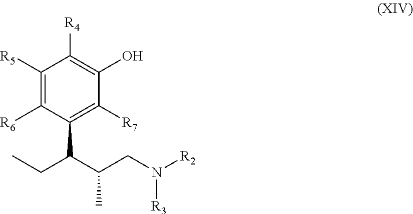

or a pharmaceutically acceptable salt or solvate thereof, and the compound obtained or obtainable by said process, the method comprising the steps (i) to (iii) as described above, wherein step (i) comprises
(a) providing a compound of formula (I)

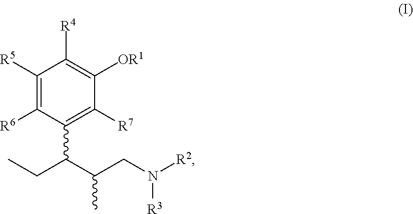

preferably

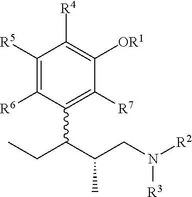

comprising of a diastereomeric mixture of the compounds of formula (Ia) and formula (Ib)

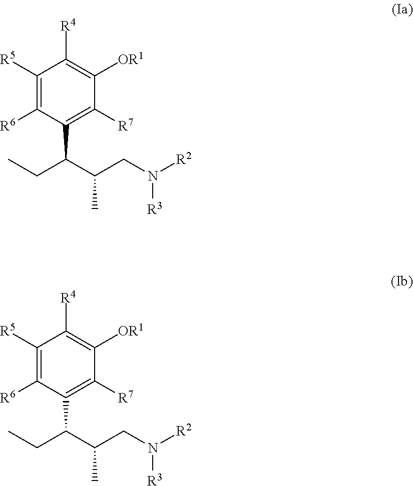

(b) forming an acid salt, preferably a tartaric acid salt, (T*) of at least part of the compound of formula (I) by treating the compound of formula (I) with a chiral acid, preferably a single stereoisomer of a chiral acid, more preferably with a tartaric acid, more preferably a single stereoisomer of a tartaric acid, wherein the tartaric acid is preferably selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and a mixture of two or more thereof, in a suitable solvent, and precipitating, preferably crystallizing, at least part of the acid salt (T*) formed, thereby obtaining a mixture comprising the precipitated, preferably crystallized acid salt (T) and the solvent;
(c) preferably separating the precipitated, preferably crystallized, acid salt (T) of the compound of formula (I) from the mixture obtained in (b),
   wherein the acid salt (T) of the compound of formula (I) contains at least 90% by weight of the acid salt of the compound of formula (Ia) based on the total weight of the acid salt (T) of the compound of formula (I).

Preferably, the compound of formula (XIV) has the structure

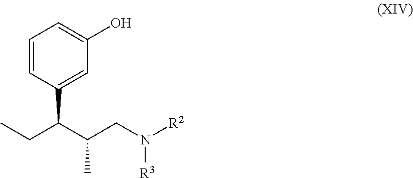

wherein R² and R³ are both methyl.

The term “pharmaceutically acceptable salts” refers to salts prepared from pharmaceutically acceptable, preferably non-toxic, bases or acids including mineral or organic acids or organic or inorganic bases. Such salts are also known as acid addition and base addition salts. Acids commonly employed from acid addition salts are inorganic acids such as hydrochloric acid, hydrobromic acid, hydroiodic acid, sulfuric acid, phosphoric acid and the like, and organic acids such as para-toluene sulfonic acid, methane sulfonic acid, oxalic acid, para-bromophenylsulfonic acid, carbonic acid, succinic acid, citric acid, benzoic acid, acidic acid and the like. Examples of pharmaceutically acceptable salts are sulfates, pyrosulfates, bisulfates, sulfites, bisulfites, phosphates, monohydrogenphosphates, dihydrogenphosphates, metaphosphates, pyrophosphates, bromides, iodides, acetates, propionates, dicanoates, caprolates, acrylates, formates hydrochlorides, dihydrochlorides, isobutyrates, caproates, heptanoates, propiolates, oxalates, malonates, succinates, suberates, sebacates, fumarates, maleates, butin-1,4-dioates, hexin-1,6-dioates, benzoates, chlorobenzoates, methylbenzoates, hydroxybenzoates, methoxybenzoates, phthalates, xylenesulfates, phenylacetates, phenylpropionates, phenylbutyrates, citrates, lactates, gamma-hydroxybutyrrates, glycolates, tartrates, methanesulfonates, propanesulfonates, naphthalene-1-sulfonates, naphthalene-2-sulfonates, mandelates and salts derived from other primary, secondary or tertiary amines including amines such as arginines, betaines, caffeines, cholines, N,N′-dibenzylethylenediamines, diethyleneethylamines, 2-diethylaminoethanols, 2-dimethylaminoethanols, ethanolamines, ethylenediamines, N-ethylmorpholines, N-ethylpiperidines, cocamines, glucosamines, histidines, hydrabamines, isopropylamines, lysines, methylglucamines, morpholines, piperazines, piperidines, polyamine resins, purines, thiobromines, triethylamines, trimethylamines, tripropylamines, tromethamines and the like. Preferred pharmaceutically acceptable acid addition salts are those formed with mineral acids such as hydrochloric acid and hydrobromic acid and those formed with organic acids such as malic acid and methanesulfonic acid. Further salts derived from inorganic bases including aluminum, ammonium, calcium, copper, ferric, ferrous, lithium, magnesium, manganic, manganous, potassium, sodium, zinc salts and the like should be mentioned.

Step (iii)

The conversion of the group —OR¹ to OH can be carried out by any method known to those skilled in the art. Preferably, (ii) is carried out by reacting the compound according to (i) with methionine in methanesulfonic acid or HBr, preferably with methionine in methanesulfonic acid.

### Step (iv)

The provision in step (iv) of the pharmaceutically acceptable salt or solvate of the compound of formula (XIV) of (ii) or (iii) is carried out by the methods known in the art.

The compound of formula (XIV) or the pharmaceutically acceptable salt thereof is preferably isolated. If such isolation is carried out, this may be carried out by any method known to those skilled in the art. Such isolation may comprise one or more stages wherein preferably at least one stage comprises a purification, such as an extraction and/or a precipitation and/or filtration. Preferably, the compound of formula (XIV) is precipitated and filtered. Preferably, the compound of formula (XIV) is obtained in crystalline form.

Further, the present invention also relates to use of an acid salt, preferably a tartaric acid salt (T), as described above, for the preparation of a compound of formula (XIV) or a pharmaceutically acceptable salt or solvate thereof), the compound preferably having the structure

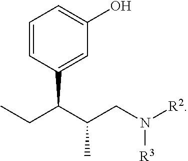

The present invention is further illustrated by the following embodiments and combinations of embodiments as indicated by the respective dependencies and references:
1. A process for the preparation of a compound of formula (I)

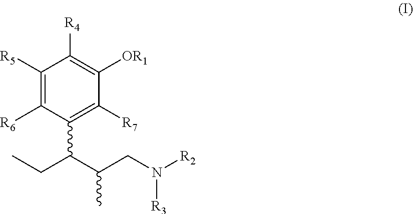

the process comprising
(a1) providing a compound of formula (II)

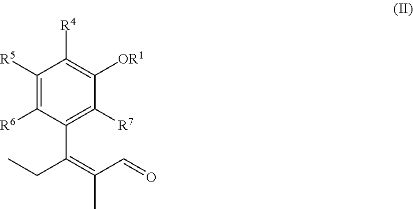

(a2) reacting the compound of formula (II) with an amine HN(R²)(R³) and reducing the resulting reaction product with hydrogen in the presence of a catalyst, preferably in the presence of Pd/C,
to give the compound of formula (I).
2. The process of embodiment 1 comprising
   (a1) providing a compound of formula (II)

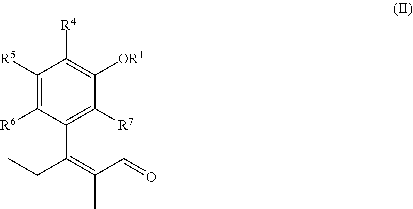

(a2) reacting the compound of formula (II) with an amine HN(R²)(R³) in the presence of a reducing agent, preferably a borohydride, thereby forming a compound of formula (III)

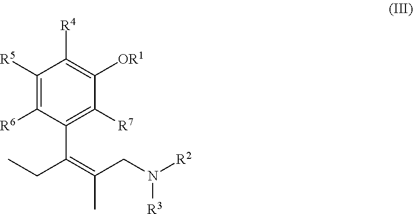

(a3) optionally isolating the compound of formula (III),
(a4) reducing the compound of formula (III) with hydrogen in the presence of a catalyst, preferably Pd/C,
to give the compound of formula (I).
3. The process of embodiment 1 or 2, wherein R⁴, R⁵, R⁶ and R⁷ are H.
4. The process of any one of embodiments 1 to 3, wherein the compound of formula II has the structure (IIa) or (IIb) or consists of a mixture of (IIa) and (IIb)

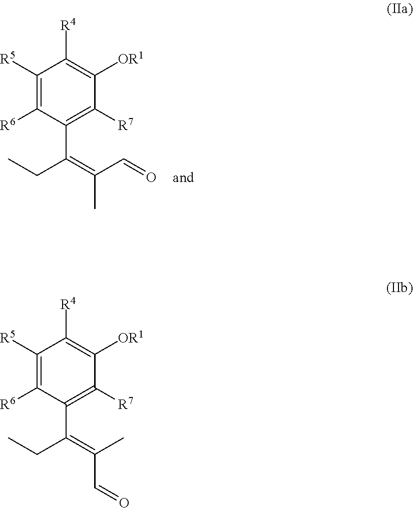

5. The process according to any of embodiments 1 to 4, wherein the compound of formula III has the structure (lla) or (lllb) or consists of a mixture of (lla) and (IIIb)

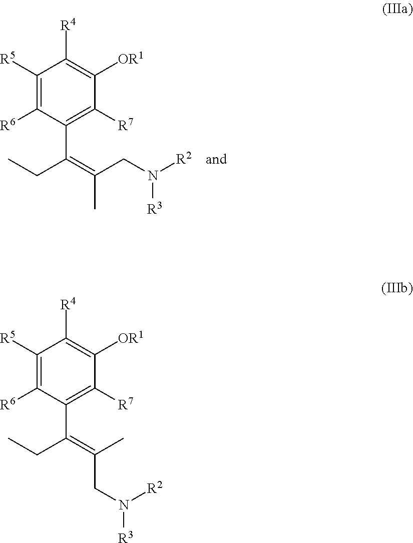

6. The process according to any one of embodiments 1 to 5, wherein the reaction in (a2) is a reductive amination.
7. The process of any one of embodiments 1 to 6, wherein step (a1) comprises
   (a1.1) providing a compound of formula (IV)

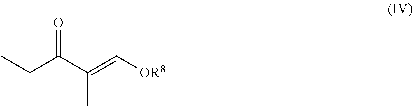

wherein R⁸ is selected from the group consisting of methyl, ethyl, isopropyl, ter.-butyl and —CH(isopropyl)₂, preferably wherein R⁸ is —CH(isopropyl)₂,
(a1.2) reacting the compound of formula (IV) with an organometallic compound of formula (V),

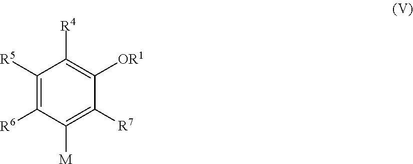

wherein M is selected from the group consisting of Li, MgBr, CuMgBr, CuLi and

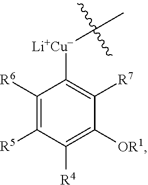

to give the compound of formula (II)
8. The process of embodiment 7, wherein M is Li.
9. The process of embodiment 7 or 8, wherein the reaction in (a1.2) is carried out in a solvent selected from the group consisting of glyme, diglyme and BF₃-etherate, preferably in glyme.
10. The process of embodiment 9, wherein the organometallic compound is prepared by reacting a compound of formula (VI)

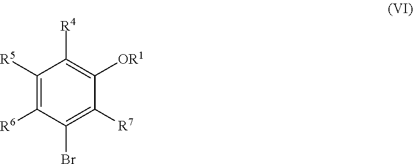

with nBuLi, preferably at a temperature of less than −70° C.
11. The process of any one of embodiments 7 to 10, wherein (a1.1) comprises
   (a1.1.1) providing a compound of formula (VI)

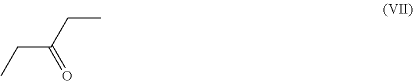

(a1.1.2) reacting the compound of formula (VII) in the presence of sodium hydride with HC(O)OR⁹ to give a compound of formula (VIII) or a sodium salt thereof, wherein R⁹ is preferably a substituted or unsubstituted alkyl or cycloalkyl group,

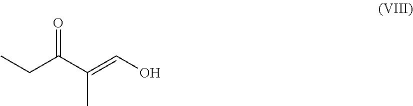

(a1.1.3) reacting the compound of formula (VIII) with R⁸—OH, wherein R⁸ is selected from the group consisting of methyl, ethyl, isopropyl, ter.-butyl and —CH(isopropyl)₂
   to give the compound of formula (IV).
12. The process of embodiment 11, wherein the reaction in (a1.1.2) is carried out in a solvent selected from the group consisting of toluene, xylol and n-heptane.
13. The process of embodiment 11 or 12, wherein the reaction in (a1.1.3) is carried out in R⁸—OH as solvent, wherein R⁸ is selected from the group consisting of methyl, ethyl, isopropyl, ter.-butyl and —CH(isopropyl)₂.
14. The process of any one of embodiments 11 to 13, wherein the reaction in (a1.1.3) is carried out in the presence of p-toluene sulfonic acid.
15. The process of any one of embodiments 1 to 14, wherein the compound of formula (I) has formula (Ia)

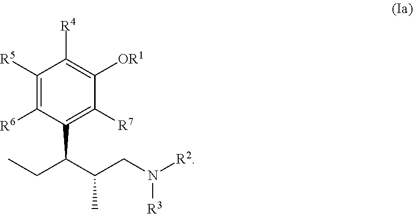

16. A process for the preparation of a compound of formula (I),

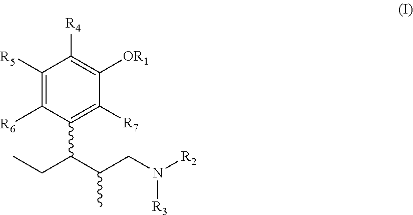

the process comprising
   (aa) providing a compound of formula (IX)

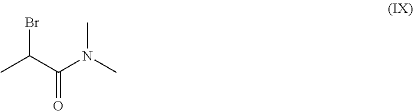

(ab) providing a compound of formula (X)

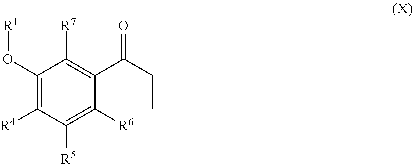

(ac) reacting the compound of formula (IX) with the compound of formula (X) in the presence of Zn to give a compound of formula (XI)

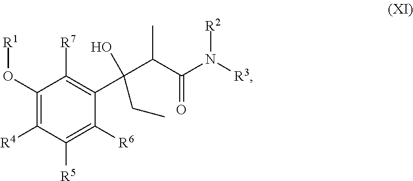

(ad) dehydrating the compound of formula (XI) to give a compound of formula (XIIa)

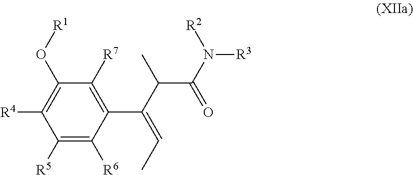

and/or a compound of formula (XIIb)

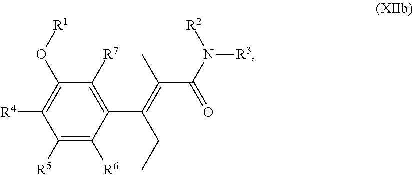

(ae) reducing the compound of formula (XIIa) and/or (XIIb),
to give the compound of formula (I).
17. The process of embodiment 16, wherein the reaction in (ad) is carried out by reacting the compound of formula (XI) with sulfuric acid.
18. The process of embodiment 16 or 17, wherein (ae) comprises
   (ae1) reducing the compound of formula (XIIa) and/or (XIIb) to give a compound of formula (XIIa1)

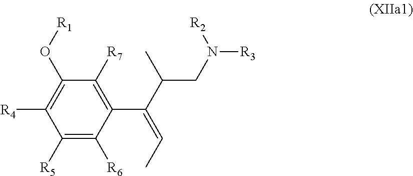

and/or a compound of formula (XIIb1)

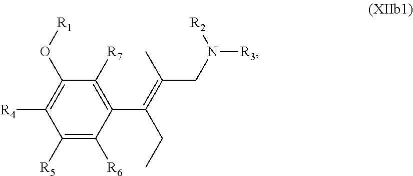

(ae2) reducing the compound of formula (XIIa1) and/or (XIIb1)
to give the compound of formula (I).
19. The process of any one of embodiments 16 to 18, wherein the compound of formula (IX) in (aa) is provided by reacting a compound of formula (XIII)

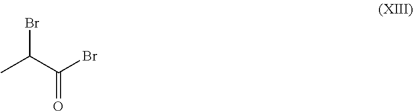

with HN(R²)(R³).
20. The process of any one of embodiments 16 to 19, wherein the compound of formula (X) is provided by reacting 1-bromo-3-methoxybenzene with propanoic anhydride, preferably via a Grignard reaction.
21. The process of any one of embodiments 16 to 20, wherein the compound of formula (I) has formula (Ia)

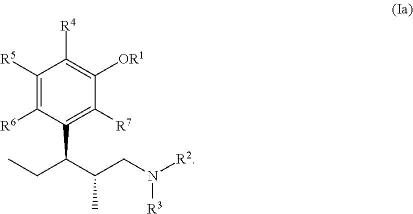

22. The process of any one of embodiments 1 to 21, wherein R⁴, R⁵, R⁶ and R⁷ are H, the compound of formula (I) having the structure

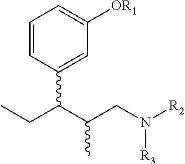

preferably

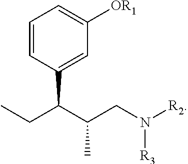

23. The process of any one of embodiments 1 to 22, wherein R¹ is selected from the group consisting of methyl, ethyl, propyl, isopropyl and benzyl, preferably wherein R¹ is methyl.
24. The process of any one of embodiments 1 to 23, wherein R² and R³, are both alkyl groups, preferably, independently of each other, selected from the group consisting of methyl, ethyl, propyl and isopropyl, more preferably, R² and R³, are methyl.
25. The process of any of embodiments 1 to 24, which further comprises the process for the preparation of an acid salt (T), preferably of a tartaric acid salt (T), of a compound of formula (I)

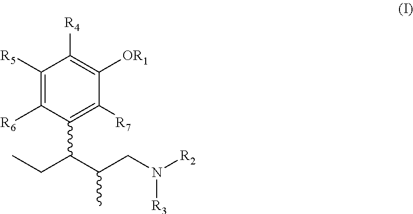

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl,
and wherein the acid salt (T) is the salt of a chiral acid, preferably the salt of a single stereoisomer of a chiral acid, preferably wherein the chiral acid is a tartaric acid,
more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof,
wherein the process comprises
(a) providing a compound of formula (I)

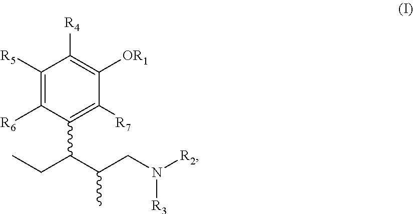

comprising a diastereomeric mixture of the compounds of formula (Ia) and formula (Ib)

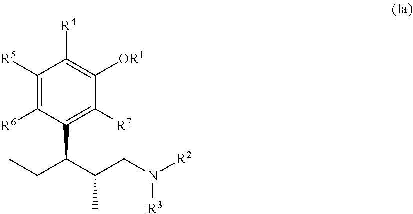

and

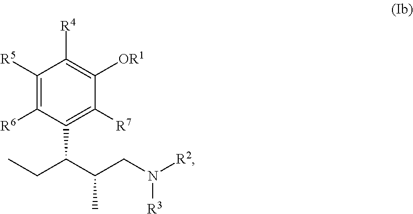

wherein the providing is according to any of embodiments 1 to 22
(b) forming an acid salt (T*), preferably a tartaric acid salt (T*), of at least part of the compound of formula (I) by treating the compound of formula (I) with a chiral acid, preferably a single stereoisomer of a chiral acid, more preferably with a tartaric acid, more preferably a single stereoisomer of a tartaric acid, wherein the tartaric acid is preferably selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and a mixture of two or more thereof, in a suitable solvent, and precipitating, preferably crystallizing, at least part of the acid salt (T*) formed, thereby obtaining a mixture comprising the precipitated, preferably crystallized acid salt (T) and the solvent;
(c) preferably separating the precipitated, preferably crystallized, acid salt (T) of the compound of formula (I) from the mixture obtained in (b),
   wherein the acid salt (T) of the compound of formula (I) contains at least 90% by weight of the acid salt of the compound of formula (Ia) based on the total weight of the acid salt (T) of the compound of formula (I).
26. The process of embodiment 25, wherein the acid salt (T) of formula (I) contains at least 95% by weight, more preferably at least 96% by weight, more preferably at least 97% by weight, more preferably at least 98% by weight, more preferably at least 99% by weight, more preferably at least 99.5% by weight, more preferably at least 99.9% by weight, of the tartaric salt of the compound of formula (Ia), based on the total weight of the acid salt (T) of the compound of formula (I).
27. The process of embodiment 25 or 26, wherein the acid salt (T*) of formula (I) contains 10 to 70% % by weight of the acid salt of the compound of formula (Ia), based on the total weight of the acid salt (T*) of the compound of formula (I).
28. The process of any one of embodiments 25 or 26 wherein the acid salt (T) of the compound of formula (I) consist of the acid salt of the compound of formula (Ia)

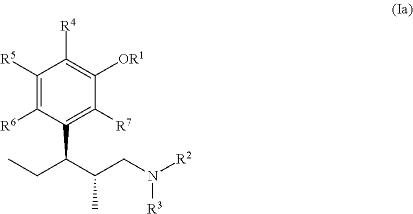

29. The process of any of embodiments 25 to 28, wherein the solvent is selected from the group consisting of ethyl acetate, acetone, isopropanol, methanol, water, formic acid ethyl ester, isopropyl acetate, propyl acetate, butyl acetate and mixtures of two or more thereof.
30. The process of any one of embodiments 25 to 29, wherein step (b) comprises dissolving the compound of formula (I) in the suitable solvent and adding a solution of the acid dissolved in a further solvent to the solution, wherein the further solvent and the suitable solvent are preferably the same, more preferably ethyl acetate.
31. The process of embodiment 30, wherein the compound of formula (I) is dissolved in the suitable solvent and the mixture is heated to a temperature in the range of from 30 to 60° C., prior to the addition of the acid.
32. The process of any one of embodiments 25 to 31, wherein the crystallizing in (b) is carried out at a temperature in the range of from 0 to 60° C., wherein the temperature is preferably continuously or stepwise decreased during step b).
33. The process of any one of embodiments 25 to 32, wherein the compound of formula (I) in (a) consists of a diastereomeric mixture of compounds of formula (Ia) and formula (Ib), and optionally the compounds (Ic) and/or (Id), the mixture comprising the compounds of formula (Ia) and (Ib) in a molar ratio in the range of from 0.2 to 1.2, preferably 0.4 to 1.
34. The process of any one of embodiments 25 to 33, wherein the separating in (c) is carried out by centrifugation or filtration, preferably filtration.
35. The process of any one of embodiments 25 to 34, wherein step (a) comprises
   (a1) providing a compound of formula (II)

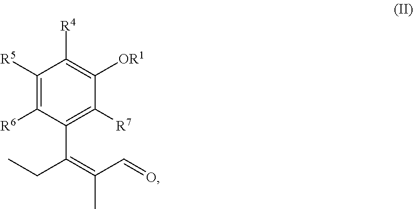

(a2) reacting the compound of formula (II) with an amine HN(R²)(R³) and reducing the resulting reaction product with hydrogen in the presence of a catalyst, preferably in the presence of Pd/C,
to give the compound of formula (I).
36. The process of embodiment 35, wherein R⁴, R⁵, R⁶ and R⁷ are H, the compound of formula (II) having the structure

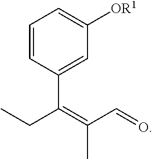

37. A process for the preparation of an acid salt (T), preferably of a tartaric acid salt (T), of a compound of formula (I)

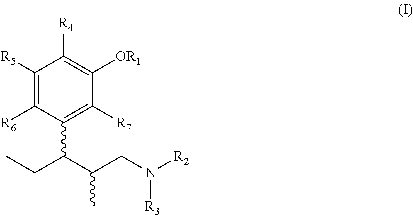

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl,
and wherein the acid salt (T) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof,
wherein the process comprises
(a) providing a compound of formula (I)

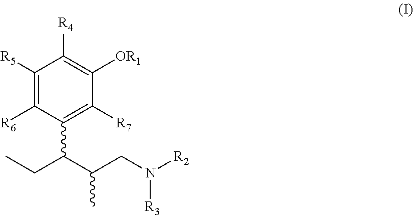

comprising a diastereomeric mixture of the compounds of formula (Ia) and formula (Ib)

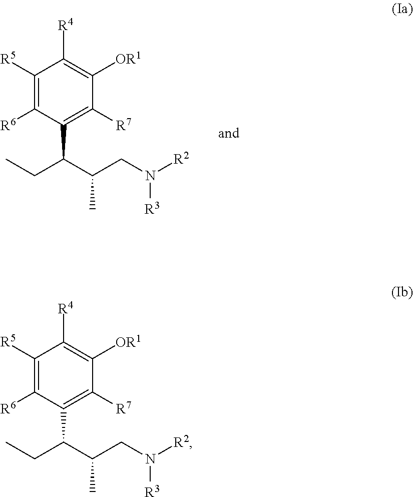

wherein the providing is by a process according to any one of embodiments 1 to 24,
(b) forming an acid salt (T*), preferably a tartaric acid salt (T*), of at least part of the compound of formula (I) by treating the compound of formula (I) with a chiral acid, preferably a single stereoisomer of a chiral acid, more preferably with a tartaric acid, more preferably a single stereoisomer of a tartaric acid, wherein the tartaric acid is preferably selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and a mixture of two or more thereof, in a suitable solvent, and precipitating, preferably crystallizing, at least part of the acid salt (T*) formed, thereby obtaining a mixture comprising the precipitated, preferably crystallized acid salt (T) and the solvent;
(c) preferably separating the precipitated, preferably crystallized, acid salt (T) of the compound of formula (I) from the mixture obtained in (b),
   wherein the acid salt (T) of the compound of formula (I) contains at least 90% by weight of the acid salt of the compound of formula (Ia) based on the total weight of the acid salt (T) of the compound of formula (I).
38. A process for the preparation of an acid salt (T), preferably of a tartaric acid salt (T), of a compound of formula (I)

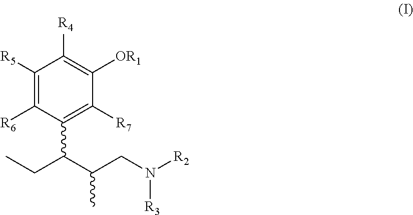

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl,
and wherein the acid salt (T) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof,
wherein the process comprises
(a) providing a compound of formula (I)

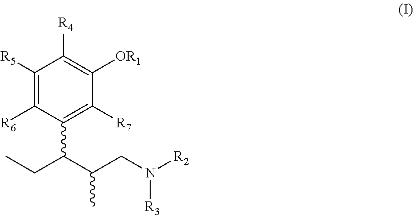

comprising a diastereomeric mixture of the compounds of formula (Ia) and formula (Ib)

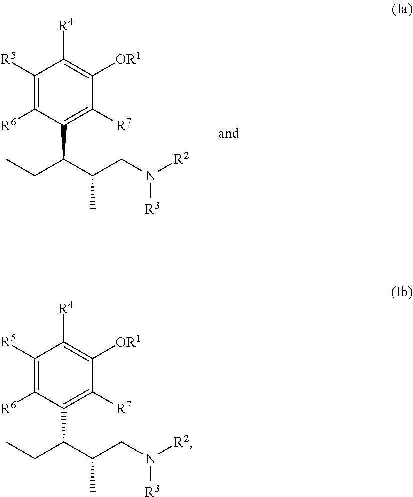

(b) forming an acid salt (T*), preferably a tartaric acid salt (T*), of at least part of the compound of formula (I) by treating the compound of formula (I) with a chiral acid, preferably a single stereoisomer of a chiral acid, more preferably with a tartaric acid, more preferably a single stereoisomer of a tartaric acid, wherein the tartaric acid is preferably selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and a mixture of two or more thereof, in a suitable solvent, and precipitating, preferably crystallizing, at least part of the acid salt (T*) formed, thereby obtaining a mixture comprising the precipitated, preferably crystallized acid salt (T) and the solvent;
(c) preferably separating the precipitated, preferably crystallized, acid salt (T) of the compound of formula (I) from the mixture obtained in (b),
   wherein the acid salt (T) of the compound of formula (I) contains at least 90% by weight of the acid salt of the compound of formula (Ia) based on the total weight of the acid salt (T) of the compound of formula (I).
39. The process of embodiment 37 or 38, wherein the acid salt (T) of formula (I) contains at least 95% by weight, more preferably at least 96% by weight, more preferably at least 97% by weight, more preferably at least 98% by weight, more preferably at least 99% by weight, more preferably at least 99.5% by weight, more preferably at least 99.9% by weight, of the tartaric salt of the compound of formula (Ia), based on the total weight of the acid salt (T) of the compound of formula (I).
40. The process of any of embodiments 37 to 39, wherein the acid salt (T*) of formula (I) contains 10 to 70% % by weight of the acid salt of the compound of formula (Ia), based on the total weight of the acid salt (T*) of the compound of formula (I).
41. The process of any one of embodiments 37 to 40, wherein the acid salt (T) of the compound of formula (I) consist of the acid salt of the compound of formula (Ia)

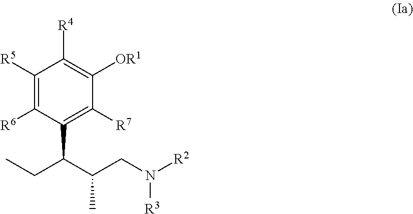

42. The process of any one of embodiments 37 to 41, wherein R⁴, R⁵, R⁶ and R⁷ are H, the compound of formula (I) having the structure

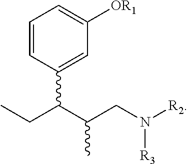

43. The process of any one of embodiments 37 to 42, wherein R¹ is selected from the group consisting of methyl, ethyl, propyl, isopropyl and benzyl, preferably wherein R¹ is methyl.
44. The process of any one of embodiments 37 to 43, wherein R² and R³, are both alkyl groups, preferably, independently of each other, selected from the group consisting of methyl, ethyl, propyl and isopropyl, more preferably, R² and R³, are methyl.
45. The process of any one of embodiments 37 to 44, wherein the solvent is selected from the group consisting of ethyl acetate, acetone, isopropanol, methanol, water, formic acid ethyl ester, isopropyl acetate, propyl acetate, butyl acetate and mixtures of two or more thereof.
46. The process of any one of embodiments 37 to 45, wherein step (b) comprises dissolving the compound of formula (I) in the suitable solvent and adding a solution of the acid dissolved in a further solvent to the solution, wherein the further solvent and the suitable solvent are preferably the same, more preferably ethyl acetate.
47. The process of embodiment 46, wherein the compound of formula (I) is dissolved in the suitable solvent and the mixture is heated to a temperature in the range of from 30 to 60° C., prior to the addition of the acid.
48. The process of any one of embodiments 37 to 47, wherein the crystallizing in (b) is carried out at a temperature in the range of from 0 to 60° C., wherein the temperature is preferably continuously or stepwise decreased during step b).
49. The process of any one of embodiments 37 to 48, wherein the compound of formula (I) in (a) consists of a diastereomeric mixture of compounds of formula (Ia) and formula (Ib), and optionally the compounds (Ic) and/or (Id), the mixture comprising the compounds of formula (Ia) and (Ib) in a molar ratio in the range of from 0.2 to 1.2, preferably 0.4 to 1.
50. The process of any one of embodiments 37 to 49, wherein the separating in (c) is carried out by centrifugation or filtration, preferably filtration.
51. The process of any one of embodiments 37 to 50, wherein step (a) comprises
   (a1) providing a compound of formula (II)

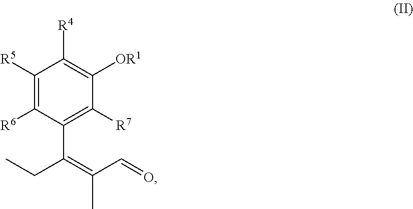

(a2) reacting the compound of formula (II) with an amine HN(R²)(R³) and reducing the resulting reaction product with hydrogen in the presence of a catalyst, preferably in the presence of Pd/C,
to give the compound of formula (I).
52. The process of embodiment 51, wherein R⁴, R⁵, R⁶ and R⁷ are H, the compound of formula (II) having the structure

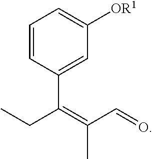

53. The process of any one of embodiments 37 to 50, wherein step (a) comprises
   (a1) providing a compound of formula (II)

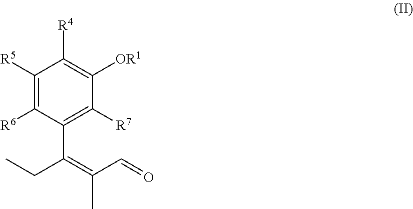

(a2) reacting the compound of formula (II) with HN(R²)(R³) in the presence of a reducing agent, preferably a borohydride, thereby forming a compound of formula (III)

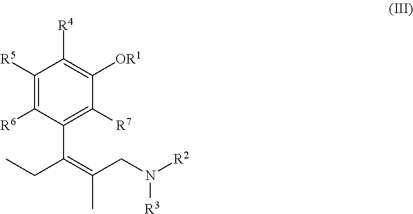

(a3) optionally isolating the compound of formula (III),
(a4) reducing the compound of formula (III) with hydrogen in the presence of a catalyst, preferably Pd/C,
to give the compound of formula (I).
54. The process of embodiment 53, wherein R⁴, R⁵, R⁶ and R⁷ are H.
55. The process of any one of embodiments 51 to 54, wherein the compound of formula (II) has the structure (IIa) or (IIb) or consists of a mixture of (IIa) and (IIb)

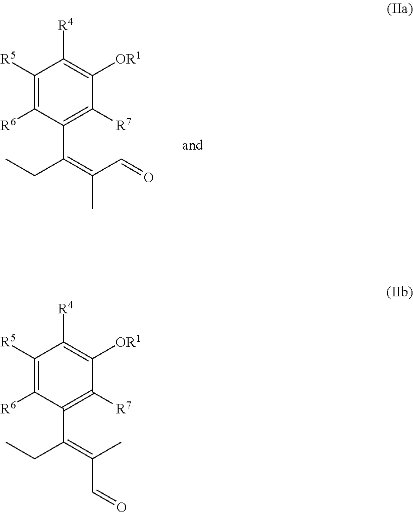

56. The process of embodiment 53 or 54, wherein the compound of formula III has the structure (IIIa) or (IIIb) or consists of a mixture of (IIIa) and (IIIb)

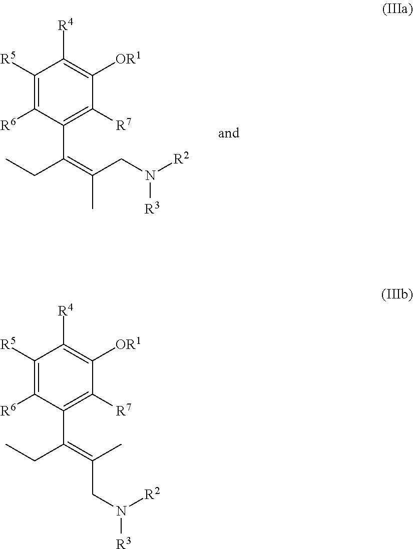

57. The process of any one of embodiments 53 to 55, wherein the reaction in (a2) is a reductive amination.
58. The process of any one of embodiments 51 to 57, wherein step (a1) comprises
   (a1.1) providing a compound of formula (IV)

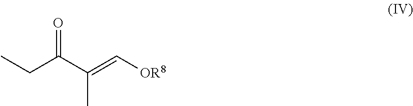

wherein R⁸ is selected from the group consisting of methyl, ethyl, isopropyl, ter.-butyl and —CH(isopropyl)₂, preferably wherein R⁸ is —CH(isopropyl)₂,
(a1.2) reacting the compound of formula (IV) with an organometallic compound of formula (V),

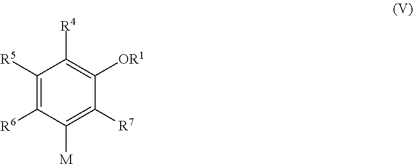

wherein M is selected from the group consisting of Li, MgBr, CuMgBr, CuLi and

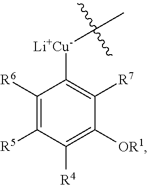

to give the compound of formula (II)
59. The process of embodiment 58, wherein M is Li.
60. The process of embodiment 58 or 59, wherein the reaction in (a1.2) is carried out in a solvent selected from the group consisting of glyme, diglyme and BF₃-etherate, preferably in glyme.
61. The process of embodiment 59 wherein the organometallic compound is prepared by reacting a compound of formula (VI)

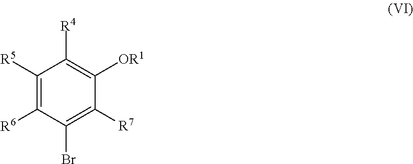

with nBuLi, preferably at a temperature of less than −70° C.
62. The process of any one of embodiments 58 to 61, wherein (a1.1) comprises
   (a1.1.1) providing a compound of formula (VII)

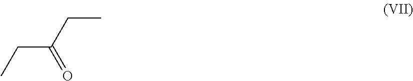

(a1.1.2) reacting the compound of formula (VII) in the presence of sodium hydride with HC(O)OR⁹ to give a compound of formula (VIII) wherein R⁹ is preferably a substituted or unsubstituted alkyl or cycloalkyl group, or a sodium salt thereof

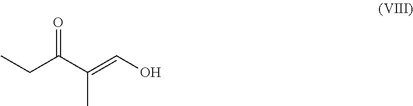

(a1.1.3) reacting the compound of formula (VIII) with R⁸—OH, wherein R⁸ is selected from the group consisting of methyl, ethyl, isopropyl, ter.-butyl and —CH(isopropyl)₂
to give the compound of formula (IV).
63. The process of embodiment 62, wherein the reaction in (a1.1.2) is carried out in a solvent selected from the group consisting of toluene, xylol and n-heptane.
64. The process of embodiment 62 or 63, wherein the reaction in (a1.1.3) is carried out in R⁸—OH as solvent, wherein R⁸ is selected from the group consisting of methyl, ethyl, isopropyl, ter.-butyl and —CH(isopropyl)₂.
65. The process of any one of embodiments 62 to 64, wherein the reaction in (a1.1.3) is carried out in the presence of p-toluene sulfonic acid.
66. The process of any one of embodiments 37 to 50, wherein (a) comprises
   (aa) providing a compound of formula (IX)

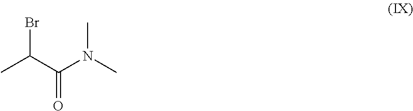

(ab) providing a compound of formula (X)

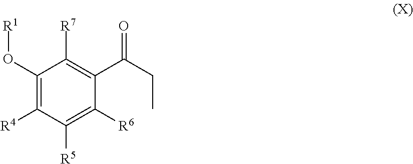

(ac) reacting the compound of formula (IX) with the compound of formula (X) in the presence of Zn to give a compound of formula (XI)

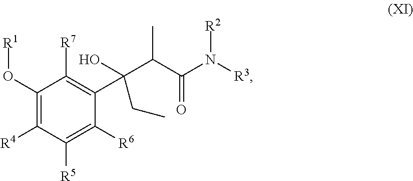

(ad) dehydrating the compound of formula (XI) to give a compound of formula (XIIa)

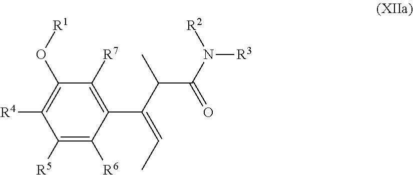

and/or a compound of formula (XIIb)

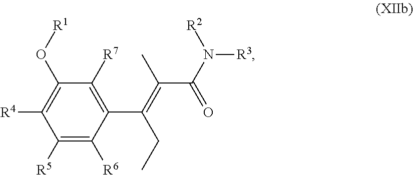

(ae) reducing the compound of formula (XIIa) and/or (XIIb),
to give the compound of formula (I).
67. The process of embodiment 66, wherein the reaction in (ad) is carried out by reacting the compound of formula (XI) with sulfuric acid.
68. The process of embodiment 66 or 67, wherein (ae) comprises
   (ae1) reducing the compound of formula (XIIa) and/or (XIIb) to give a compound of formula (XIIa1)

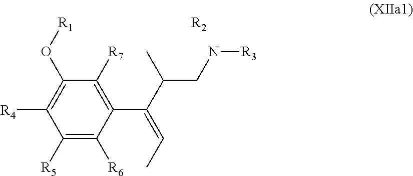

and/or a compound of formula (XIIb1)

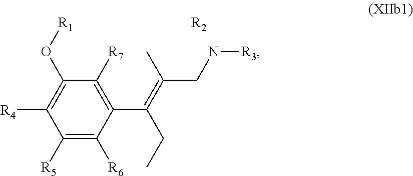

(ae2) reducing the compound of formula (XIIa1) and/or (XIIb1)
to give the compound of formula (I).
69. The process of any one of embodiments 66 to 68, wherein the compound of formula (IX) in (aa) is provided by reacting a compound of formula (XIII)

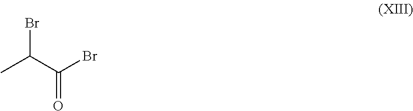

with HN(R²)(R³).
70. The process of any one of embodiments 66 to 69, wherein the compound of formula (X) is provided by reacting 1-bromo-3-methoxybenzene with propanoic anhydride, preferably via a Grignard reaction.
71. A process for the preparation of a compound of formula (XIV)

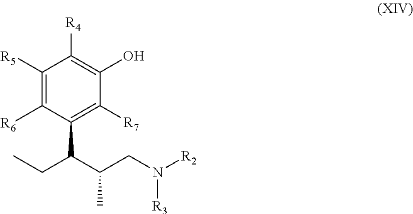

or a pharmaceutically acceptable salt or solvate thereof, comprising
(i) providing an acid salt, preferably a tartaric acid salt, (T) of a compound of formula (I) of any one of embodiments 37 to 70,
(ii) transforming the group —OR¹ to OH,
(iii) optionally purifying the compound obtained in (ii)
to give the compound of formula (XIV)
(iv) optionally preparing pharmaceutically acceptable salt or solvate of compound (XIV) of (ii) or (iii).
72. The process of embodiment 71, wherein the compound of formula (XIV) has the structure

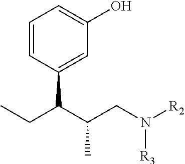

73. The process according embodiment 71 or 72, wherein (ii) is carried out by reacting the compound according to (i) with methionine in methanesulfonic acid or HBr, preferably with methionine in methanesulfonic.
74. A compound of formula (XIV) obtained or obtainable by a process according to any one of embodiments 72 or 73.
75 An acid salt (T), preferably a tartaric acid salt (T), of a compound of formula (I)

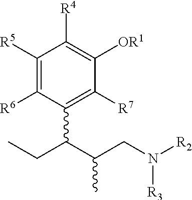

preferably,

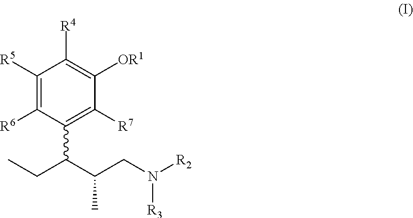

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl,
and wherein the acid salt (T) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof,
and wherein the acid salt (T) of the compound of formula (I) contains at least 90% by weight of the acid salt of the compound of formula (Ia) based on the total weight of the acid salt of the compound of formula.
76. The acid salt (T) of embodiment 75 wherein the acid salt contains at least 95% by weight, more preferably at least 96% by weight, more preferably at least 97% by weight, more preferably at least 98% by weight, more preferably at least 99% by weight, more preferably at least 99.5% by weight, more preferably at least 99.9% by weight, of the acid salt of the compound of formula (Ia)

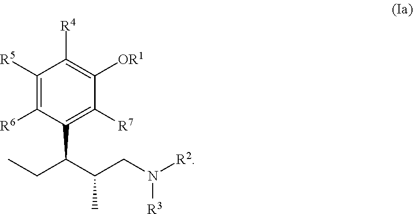

based on the total weight of the acid salt (T) of the compound of formula (I).
77. The acid salt (T) of embodiment 75, wherein the wherein the acid salt consist of the acid salt of the compound of formula (Ia).
78. The acid salt of any one of embodiments 75 to 77, wherein R¹ is selected from the group consisting of methyl, ethyl, propyl, isopropyl and benzyl, preferably wherein R¹ is methyl.
79. The acid salt (T) of any one of embodiments 75 to 78, wherein R⁴, R⁵, R⁶ and R⁷ are H.
80. The acid salt (T) of any one of embodiments 75 to 79, wherein compound (I) has the structure

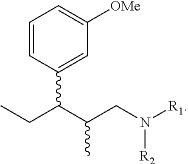

81. The acid salt (T) of any one of embodiments 75 to 80, wherein R² and R³ are methyl.
82. The acid salt (T) of any one of embodiments 75 to 81, wherein compound (I) has the structure

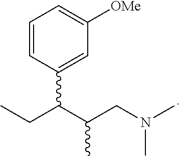 [SMILES: CCC(c1cccc(OC)c1)C(C)CN(C)C]

83. The acid salt (T) of any one of embodiments 75 to 82, wherein compound (Ia) has the structure

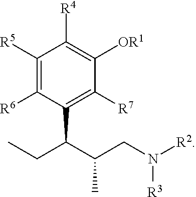

84. The acid salt (T) of any one of embodiments 75 to 83, wherein compound (Ia) has the structure

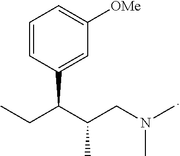 [SMILES: CC[C@@H](c1cccc(OC)c1)[C@@H](C)CN(C)C]

85. An acid salt (T) obtained or obtainable by a method of any one of embodiments 1 to 70.
86. An acid salt (T), preferably a tartaric acid salt (T*), of a compound of formula (I)

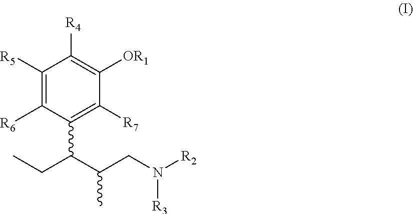

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl,
and wherein the acid salt (T*) is the salt of a chiral acid, preferably the salt of a single stereoisomer of a chiral acid, preferably wherein the chiral acid is a tartaric acid, more preferably wherein the tartaric acid is selected from the group consisting of 2,3-Ditoluoyl tartaric acid, 2,3-Dibenzoyl tartaric acid, 2,3-Dianisoyl tartaric acid, 2,3-Dibenzoyl tartaric acid mono(dimethylamide) and mixtures of two or more thereof,
wherein the acid salt (T*) of formula (I) contains 10 to 70% % by weight of the tartaric salt of the compound of formula (Ia),

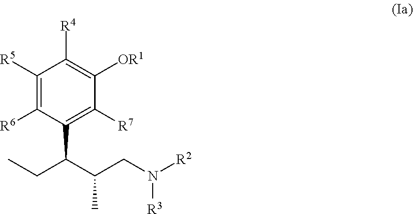

based on the total weight of the acid salt (T*) of the compound of formula (I).
87. The acid salt (T*) of embodiment 86, wherein R¹ is selected from the group consisting of methyl, ethyl, propyl, isopropyl and benzyl, preferably wherein R¹ is methyl.
88. The acid salt (T*) of embodiment 86 or 87, wherein R⁴, R⁵, R⁶ and R⁷ are H.
89. The acid salt (T*) of any one of embodiments 86 to 88, wherein compound (I) has the structure

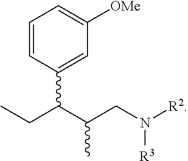

90. The acid salt (T*) of any one of embodiments 86 to 89, wherein R² and R³ are methyl.
91. The acid salt (T*) of any one of embodiments 86 to 90, wherein compound (I) has the structure

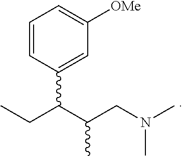 [SMILES: CCC(c1cccc(OC)c1)C(C)CN(C)C]

92. An acid salt (T*) of any one of embodiments 86 to 91, wherein the acid salt is a tartaric acid salt.
93. A compound of formula (II)

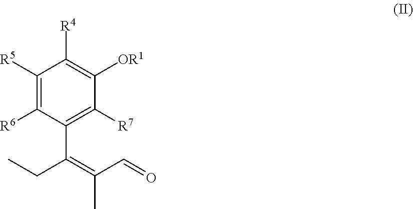

wherein R¹ is selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl.
94. The compound of embodiment 93, wherein R⁴, R⁵, R⁶ and R⁷ are H.
95. The compound of embodiment 93 or 94, wherein R¹ is selected from the group consisting of methyl, ethyl, propyl, isopropyl and benzyl, preferably wherein R¹ is methyl.
96. The compound of any one of embodiments 93 to 95, having the structure

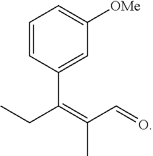

97. The compound of any one of embodiments 93 to 96, having the structure (IIa) or (IIb) or consisting of a mixture of (IIa) and (IIb)

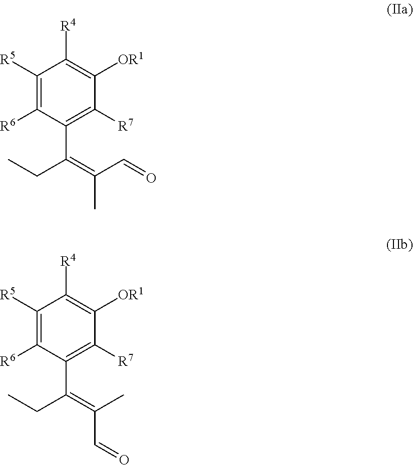

98. The compound of any one of embodiments 93 to 97, wherein the compound is obtained or obtainable by a method comprising
   (a1.1) providing a compound of formula (IV)

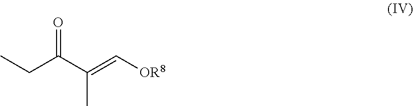

wherein R⁸ is selected from the group consisting of methyl, ethyl, isopropyl, ter.-butyl and —CH(isopropyl)₂, preferably wherein R⁸ is —CH(isopropyl)₂,
(a1.2) reacting the compound of formula (IV) with an organometallic compound of formula (V)

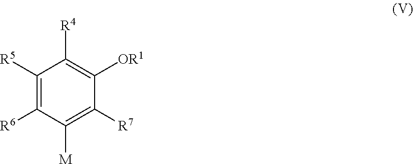

wherein M is selected from the group consisting of Li, MgBr, CuMgBr and CuLi and

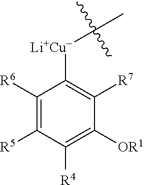

to give the compound of formula (II).
99. The compound according embodiment 98, wherein M is Li.
100. The compound according embodiment 98 or 99, wherein the reaction in step (a1.2) is carried out in a solvent selected from the group consisting of glyme, diglyme and BF₃-etherate, preferably in glyme.
101. The compound of embodiment 99, wherein the organometallic compound of formula (V) is prepared by reacting a compound of formula (VI)

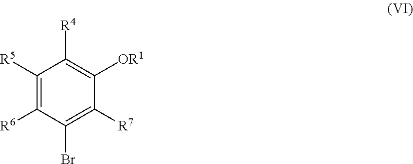

with nBuLi, preferably at a temperature of less than −70° C.
102. A compound of formula (III)

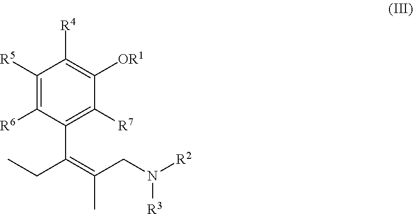

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl.
103. The compound of embodiment 102, wherein R⁴, R⁵, R⁶ and R⁷ are H.
104. The compound according embodiment 102 or 103, wherein R¹ is selected from the group consisting of methyl, ethyl, propyl, isopropyl and benzyl, preferably wherein R¹ is methyl.
105. The compound of any one of embodiments 102 to 104, wherein R² and R³, are both alkyl groups, preferably, independently of each other, selected from the group consisting of both methyl, ethyl, propyl and isopropyl, more preferably, R² and R³, are methyl.
106. The compound of any one of embodiments 102 to 105, wherein R² and R³, are both H.
107. The compound of any one of embodiments 102 to 106; wherein the compound of formula (III) has the structure (IIIa) or (IIIb) or consists of a mixture of (IIIa) and (IIIb)

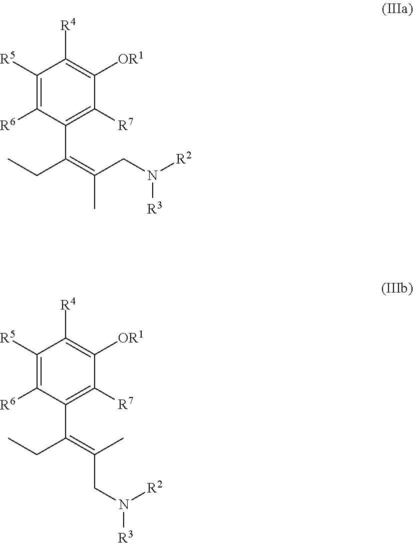

108. The compound of any one of embodiments 102 to 107; wherein the compound has the structure

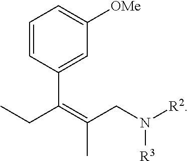

109. The compound of embodiment 107, wherein R⁴, R⁵, R⁶ and R⁷ are H.
110. A compound of formula (IV)

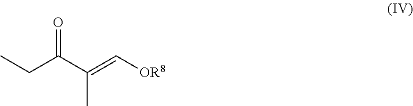

wherein R⁸ is —CH(isopropyl)₂.
111. A compound of formula (I)

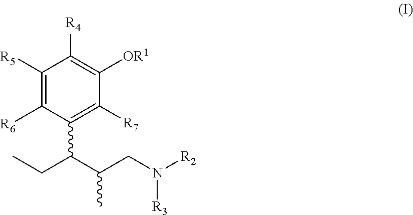

wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl.
112 The compound of embodiment 111 having the structure

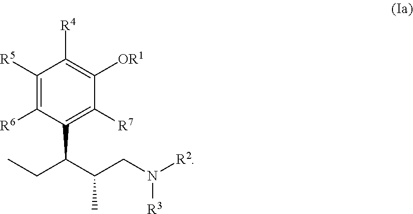

113. The compound of embodiment 111 or 112, wherein R¹ is selected from the group consisting of methyl, ethyl, propyl, isopropyl and benzyl, preferably wherein R¹ is methyl.
114. The compound of any one of embodiments 111 to 113, wherein R⁴, R⁵, R⁶ and R⁷ are H.
115. The compound of any one of embodiments 111 to 114, having the structure

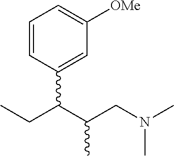 [SMILES: CCC(c1cccc(OC)c1)C(C)CN(C)C]

116. The compound of any one of embodiments 111 to 115, wherein R² and R³ are methyl.
117. Use of an acid salt (T), preferably of a tartaric acid salt (T), according to embodiments 75 to 85, for the preparation of a compound of formula (XIV) or a pharmaceutically acceptable salt or solvate thereof), the compound having the structure

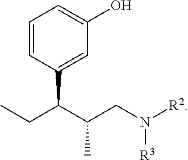

118. The process of any of embodiments 1 to 24, further comprising resolving the compound of formula (I) and obtaining a compound of formula (Ia).
119. The process of embodiment 118, wherein the compound of formula (I) has structure

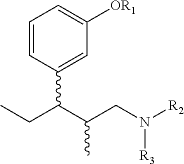

wherein preferably R¹, R² and R³ are methyl and the compound of formula (Ia) has structure

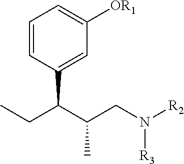

The present invention is further illustrated by the following examples.

### EXAMPLES

### Synthesis of (1E)-1-hydroxy-2-methylpent-1-en-3-one sodium salt

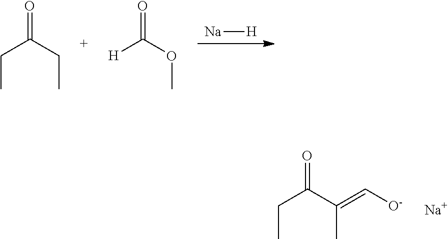

In 2 L four-necked flask (pre-dried, filled with Ar) equipped with mechanic stirred, dropping funnel 250 mL, termometer and tube with calcium chloride 26 g (1.08 mol) of sodium hydride was placed. 600 mL of toluene was added and flask placed in ice-bath. Methanol (5 mL) was added to toluene, till evolution of gas was clearly seen. Dropping funnel was charged with mixture of 3-pentanon and ethyl formate. Reagents were added to the mixture in such manner, that temperature did not rise above 10° C. Oily, sticky precipitate was formed which imparts stirring. When gas evolution ceases, toluene was replaced with 600 mL of diethyl ether and stirring was continued overnight. The following day white-off precipitate was collected via filtration and dried in rotavapour (HPLC: 100% a/a).

### Synthesis of O-Protected Ketenols

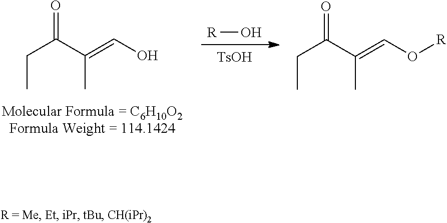

In 2 L roundbottomed flask 1-hydroxypent-1-en-3-on (96.3 g, 0.84 M) is placed. 1.2 L of isopropanol was added along with p-toluenesulfonic acid (8.0 g, 42 mM). Efficient condenser was fitted and mixture was heated for 16 hours (overnight). Solvent was evaporated on rotavapour. Crude reaction mixture was transferred to 250 mL roundbottomed flask. Distillation afforded fraction boiling at 90-95° C. Yield was 74.5 g (56.5%) of yellow oil. HPLC analysis confirmed purity of 84%.

### Synthesis of Methyl Derivative

| | | | Results | | |
|---|---|---|---|---|---|
| | | | Amount | | |
| N^{o} | Type | Reagents | [g] | Yield [%] | Purity [%] |
| 1 | E | 1-hydroxypent-1-en-3-on sodium salt | 0.75 | 36 | 95 |
| | | (2.2 g, 1.62 mmol); methyl iodide (2.3 g, | | | (HPLC) |
| | | 1.62 mmol); DMF 40 mL | | | |
| 2 | E | sodium (1E)-2-methyl-3-oxopent-1-en- | 74.6 | 58 | 98 |
| | | 1-olate 64.1 g (1.0 eq.), citric acid 120 g, | | | (HPLC) |
| | | pH = 4, separation; methyl iodide 35 mL | | | |
| | | (1.0 eq) drop wise addition at −78° C. | | | |
| | | acetone 16 h | | | |
| 3 | E | 1-hydroxypent-1-en-3-on sodium salt | 1.05 | 82 | 99 |
| | | (1.36 g. 1 mmol); p-TsOH 1 mmol; | | | (HPLC) |
| | | methanol 20 mL | | | |
| 4 | U | 1-hydroxypent-1-en-3-on (0.66 mol), | 73.3 | 86 | 60 |
| | | MeOH 300 mL, p-TsOH cat. reflux, | | | (HPLC) |
| | | overnight | | | |

### Synthesis of Ethyl Derivative

| | | | Results | | |
|---|---|---|---|---|---|
| | | | Amount | Yield | |
| N^{o} | Type | Reagents | [g] | [%] | Purity [%] |
| 1 | E | 1-hydroxypent-1-en-3-on sodium | 1.0 | 77 | 77 |
| | | salt (1.36 g, 1 mmol); p-TsOH | | | (HPLC) |
| | | 1 mmol; ethanol 20 mL | | | |

### Synthesis of Isopropyl Derivative

| | | | Results | | |
|---|---|---|---|---|---|
| | | | Amount | Yield | |
| N^{o} | Type | Reagents | [g] | [%] | Purity [%] |
| 1 | E | 1-hydroxypent-1-en-3-on sodium salt | 1.1 | 70 | 98 |
| | | (1.36 g 1 mmol); p-TsOH 1 mmol; | | | (HPLC) |
| | | isopropanol 20 mL; distilation | | | |
| 2 | E | 3.6 g of crude reaction mixture; filtered | 3.16 | 88 | 97 |
| | | through silica pad | | | (HPLC) |
| 3 | E | 10 g of reaction mixture; distillation | 4.3 + 3.4 | 43 (77) | 93 |
| | | | | | (HPLC) |

### Synthesis of diisopropylcarbinol Derivative

| | | | Results | | |
|---|---|---|---|---|---|
| | | | Amount | Yield | |
| N^{o} | Type | Reagents | [g] | [%] | Purity [%] |
| 1 | E | 1-hydroxy-2-methyl-1-en-3-one | 21.4 | 37.6 | 99 |
| | | 34 g (0.3 mol); | | | (HPLC) |
| | | 2,4-dimethyl-3-pentanol | | | |
| | | 69.7 g (0.6 mol); p-TsOH 1.7 g; | | | |
| | | toluene 250 mL | | | |

Heating of en one with t-butanol gave product is field less than 5%.

### Addition of Lithiumorganic Reagents to Carbonyl Function

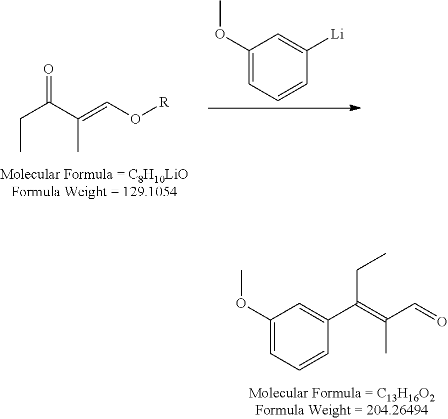

In three headed 2 L flask equipped with condenser fitted with calcium chloride containing tube, septum and termomether, bromoanisole 28.7 g (0.154 mol) was dissolved in 280 mL of anhydrous THF (distilled over sodium, with benzophenone as indicator). Flask was placed in acetone-dry ice bath and chilled to −78° C. 96 mL of nBuLi (0.154 mol) solution in hexanes (c=1.6 M) was added dropwise with syringe in such manner that temperature of reaction did not exceed −70° C. Reaction mixture was stirred for 30 minutes. Later 17.4 mL (0.141 mol) of BF₃ etherate was added immediately with syringe. Immediately solution of O-isopropyl-1-hydroxy-penten-3-on in mixture of 20 mL of glyme and 20 mL of THF was added via syringe, in such manner that reaction temperature did not exceed −70° C. Mixture was stirred for 2 hours in acetone-dry ice bath. Ice-cold solution of 13.8 g NH₄Cl (0.256 mol) in 300 mL of water was added and mixture allowed to warm up to room temperature. Mixture was transferred to separating funnel. Organic phase was separated, dried over magnesium sulfate for 10 minutes. Solvent was removed on rotavapour. Crude product was purified by chromatography using mixture of hexanes:ethyl acetate 50:1. Fraction of Rf=0.2 were collected. Yield 18.6 g (70.4%).

| | | | Results | | |
|---|---|---|---|---|---|
| | | | Amount | Yield | |
| N^{o} | Type | Reagents | [g] | [%] | Purity [%] |
| 1 | P | Bromoanisole 86.2 g (0.46 mol); nBuLi | 34.2 | 43.5 | 90 |
| | | 1.6M in hexane 288 ml (0.46 mol); | | | (HPLC) |
| | | BF₃-etherate 52.2 mL (0.42 mol); 1,2- | | | |
| | | dimethoxyethan 40 mL (0.384 mol); O- | | | |
| | | isopropyl-1-hydroxy-penten-3-on 60 g | | | |
| | | (0.384 mol); THF 1000 mL | | | |
| | | aldehyde 1 separated | — | — | — |
| | | aldehyde 2 separated | 14.3 | — | 90 |
| | | | | | (HPLC) |
| 2 | P | Bromoanisole 115 g (0.615 mol); nBuLi | 66.4 | 63.5 | 94 |
| | | 1.6M in hexane 384 mL (0.615 mol); | | | (HPLC) |
| | | BF₃-etherate 69.4 mL (0.564 mol); 1,2- | | | |
| | | dimethoxyethan 53.2 mL (0.512 mol); | | | |
| | | O-isopropyl-1-hydroxy-penten-3-on 80 g | | | |
| | | (0.512 mol); THF 1000 mL | | | |
| | | aldehyde 1 separated | 16.8 | — | 86 |
| | | | | | (HPLC) |
| | | aldehyde 2 separated | 4.4 | | 84 |
| | | | | | (HPLC) |
| 3 | P | Bromoanisole 43 g (0.23 mol); nBuLi | 25.2 | 64.3 | 93 |
| | | 1.6M in hexane 144 mL (0.23 mol); | | | (HPLC) |
| | | BF₃-etherate 26.1 mL (0.21 mol); 1,2- | | | |
| | | dimethoxyethan 20 mL (0.192 mol); O- | | | |
| | | isopropyl-1-hydroxy-penten-3-on 30 g | | | |
| | | (0.192 mol); THF 450 mL | | | |
| | | aldehyde 1 separated | — | — | — |
| | | aldehyde 2 separated | 4.95 | — | 82 |
| | | | | | (HPLC) |
| 4 | P | Bromoanisole 97.5 g (0.52 mol); nBuLi | 59.1 | 66.7 | 94 |
| | | 1.6M in hexane 325 mL (0.52 mol); | | | (HPLC) |
| | | BF₃-etherate 59 mL (0.478 mol); 1,2- | | | |
| | | dimethoxyethan 45 ml (0.43 mol); O- | | | |
| | | isopropyl-1-hydroxy-penten-3-on 67:,8 g | | | |
| | | (0.43 mol); THF 1000 ml | | | |
| | | aldehyde 1 separated | 20.1 | | 93 |
| | | | | | (HPLC) |
| | | aldehyde 2 separated | 6.8 | | 91 |
| | | | | | (HPLC) |
| 5 | P | Bromoanisole 56 g (0.30 mol); nBuLi | 27.3 | 54 | 91 |
| | | 1.6M in hexanes 187 mL (0.30 mol); | | | (HPLC) |
| | | BF₃-etherate 34 mL (0.275 mol); 1,2- | | | |
| | | dimethoxyethan 26 ml (0.25 mol); O- | | | |
| | | isopropyl-1-hydroxy-penten-3-on 38:,9 g | | | |
| | | (0.249 mol); THF 500 mL | | | |
| | | aldehyde 1 separated | 4.0 | | 75 |
| | | | | | (HPLC) |
| | | aldehyde 2 separated | 4.3 | | 84 |
| | | | | | (HPLC) |
| 6 | P | Bromoanisole 86.2 g (0.46 mol); nBuLi | 51.6 | 65.8 | 97 |
| | | 1.6M in hexanes 288 mL (0.46 mol); | | | (HPLC) |
| | | BF₃-etherate 52.2 ml (0.42 mol); 1,2- | | | |
| | | dimethoxyethan 40 ml (0.384 mol); O- | | | |
| | | isopropyl-1-hydroxy-penten-3-on 60 g | | | |
| | | (0.384 mol); THF 900 ml | | | |
| | | aldehyde 1 separated | 22.8 | | 75 |
| | | | | | (HPLC) |
| | | aldehyde 2 separated | — | — | — |
| 7 | P | Bromoanisole 56 g (0.30 mol); nBuLi | 45.7 | 90 | 90 |
| | | 1.6M in hexanes 187 ml (0.30 mol); | | | (HPLC) |
| | | BF₃-etherate 34 ml (0.275 mol); 1,2- | | | |
| | | dimethoxyethan 26 ml (0.25 mol); O- | | | |
| | | isopropyl-1-hydroxy-penten-3-on 38.9 g | | | |
| | | (0.249 mol); 450 ml | | | |
| | | aldehyde 1 separated | 17.1 | | 75 |
| | | | | | (HPLC) |
| | | aldehyde 2 separated | — | — | — |
| 8 | P | Bromoanisole 28.7 g (0.154 mol); | 18.6 | 70 | 97 |
| | | nBuLi 1.6M in hexanes 96 ml (0.154 mol); | | | (HPLC) |
| | | BF₃-etherate 17.4 ml (0.141 mol); | | | |
| | | 1,2-dimethoxyethan 13.3 ml (0.128 mol); | | | |
| | | O-isopropyl-1-hydroxy-penten-3- | | | |
| | | on 20 g (0.128 mol); THF 300 ml | | | |

### Reductive Amination of Aldehyde, Synthesis of OMe-Tapentadol

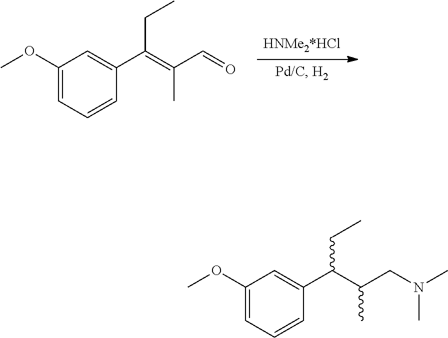

### Reductive Amination Over Palladium on Carbon.

3-(3-methoxyphenyl)-2-methylpent-2-enal (10.31 g, 0.05 mol) was dissolved in 100 ml of MeOH pure grade. Dimethyl amine hydrochloride (16.31 g, 0.2 mol) was added and mixture was stirred to dissolve salt of amine. Triethylamine (20.2 g, 0.2 mol) and wetted 10% Pd/C (0.53 g, 0.0005 mole) were added and whole mixture placed in glass cylinder, fitted with Teflon cork containing hole for plastic tube. Tube fitted with o-ring and plastic tube was fitted to cork and bottle placed inside Parr apparatus, and connected via tube with pressured hydrogen tank. Apparatus valve was set to “open”. Valve was fitted with rubber tube, connected to vacuum pump. Pump was started and when pressure dropped below 50 bar apparatus valve was set to “close”. Immediately hydrogen tank was set to “open” and bottle was pressurized slowly with hydrogen at pressure of 1 bar. Hydrogen tank was set to close as soon pressure inside bottle reached 1 bar. Bottle was shaken for 120 hours. During reaction pressure was checked every two hours during daytime and pressure kept at 1 bar. After 120 hours apparatus valve was set to“open” and whole hydrogen slowly released. Methanol from reaction mixture was removed on rotavapour (bath temperature 25° C.). Ethyl acetate (100 ml) and 1 N HCl (100 ml) were added to remaining oil. Organic phase was washed with 1 N HCl (10 ml) until no product was detected in organic phase (usually two or three washings are enough). Organic phase was discarded and aqueous phase was alkalized to pH=8 with sodium bicarbonate 1 N solution (130-140 ml). Aqueous phase was washed three times with 10 ml of ethyl acetate. Organic phase was combined, dried over magnesium sulfate. Removal of solvent yielded 0.5 g (5%) of OMe-Tapentadol with isomer ratio (S,R)+(R,R) 47%, (R,S) 33%, (S,S) 20%.

| | Hydride | | Allylic | Allylic | OMe- | | Yield/ |
|---|---|---|---|---|---|---|---|
| No | source | Base | alcohol | amine | TAP | Purity | notes |
| relative ratio of isomers | | | | (S,R) | (R,R) | (R,S) | (S,S) |
| 1 | Pd/C H₂ | TEA | — | 26% | 74% | 82% | |
| | 3 atm | | | | | | |
| | (E)-aldehyde | | | | | | |
| relative ratio of isomers | | | | 45 | 42 | | 13 |
| 2 | Pd/C H₂ | TEA | — | — | 100% | 52% | Conversion |
| | 2.36 atm | | | | | | full |
| | (Z)-aldehyde | | | | | | |
| relative ratio of isomers | | | | 47 | 37 | | 16 |
| 3 | Pd/C H₂ | TEA | — | 9% | 91% | 90% | 56% |
| | 1 atm | | | | | | |
| | (E)-aldehyde | | | | | | |
| relative ratio of isomers | | | | 48 | 40 | | 12 |
| 4 | Pd/C H₂ | TEA | — | 14% | 86% | 97% | 30% |
| | 1 atm | | | | | | |
| | (E)-aldehyde | | | | | | |
| relative ratio of isomers | | | | 45 | 41 | | 14 |
| 5 | Pd/C H₂ | TEA | — | 4% | 96% | 100% | |
| | 2.36 atm | | | | | | |
| | (Z)-aldehyde | | | | | | |

| | | | |
|---|---|---|---|
| relative ratio of isomers | 40 | 40 | 20 |

Reductive Amination, Synthesis of (2Z,E)-3-(3-methoxyphenyl)-N,N,2-trimethylpent-2-en-1-amine

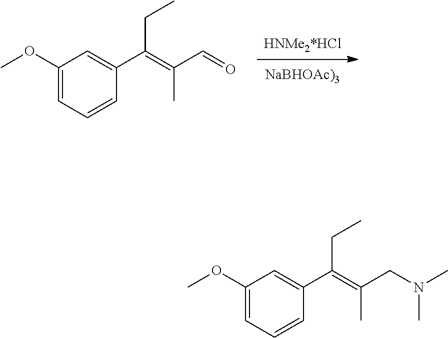

In 100 ml of chloroform pure grade, placed in 250 ml round-bottomed flask, Z-3-(3-methoxyphenyl)-2-methylpent-2-enal (4.08 g 20 mM) was dissolved. Acetic acid (2.4 g 40 mM) and dimethyl amine hydrochloride (8.16 g 100 mM) were added. Mixture was mixed with magnetic bar for 15 minutes to achieve complete dissolution of dimethyl amine hydrochloride. Sodium triacetoxyborohydride (8.5 g, 40 mM) was added and reaction mixture was allowed to react for 4 hours. HPLC analysis shown that full conversion of substrate was obtained and 58% of product formed. Solution was transferred to separating funnel (250 ml) and washed three times with 30 ml of water. Aqueous phases were combined, washed with 20 ml of ether. NaOH 4.4 g was added and mixture was washed three times with ether. Organic extracts were combined, dried over magnesium sulfate for 10 minutes. Ether was removed on rotavapour. Amine was dissolved in ether HPLC grade (50 ml) and slowly, 2.6 M solution of HCl in ether (8 ml) was added with mixing. 4.02 g (75%) of white solid was obtained via filtration and drying.

| | | | Results | | |
|---|---|---|---|---|---|
| | | | Amount | Yield | |
| N^{o} | Type | Reagents | [g] | [%] | Purity [%] |
| 1 | P | dimethylamine hydrochloride 32.58 g (0.4M) | 10.7 | 50 | 97 |
| | | acetic acid 9.6 g (0.16M) | | | (HPLC) |
| | | sodium triacetoxyborohydride 33.88 g (0.16M) | | | Z:E 97:3 |
| | | 3-(3-methoxyphenyl)-2-methylpent-2-enal | | | |
| | | 16.3 g (0.08M) [Z:E 97:3] | | | |
| | | NaOH 17.6 g (0.44M) chloroform | | | |
| 2 | P | dimethylamine hydrochloride 30.98 g (0.38M) | 11.9 | 58 | 92 |
| | | acetic acid 9.13 g (0.152M) | | | (HPLC) |
| | | sodium triacetoxyborohydride 32.21 g (0.152M) | | | Z:E |
| | | 3-(3-methoxyphenyl)-2-methylpent-2-enal | | | 31:63 |
| | | 15.5 g (0.076M) [Z:E 31:59] | | | |
| | | NaOH 16.7 g (0.418M) chloroform | | | |
| 3 | | dimethylamine hydrochloride 30.64 g (0.375M) | 12.34 | 55 | 97 |
| | | acetic acid 9.03 g (0.15M) | | | (HPLC) |
| | | sodium triacetoxyborohydride 31.9 g (0.15M) | | | Z:E |
| | | 3-(3-methoxyphenyl)-2-methylpent-2-enal | | | 22:75 |
| | | 15.3 g (0.075M) [Z:E 27:51] | | | |
| | | NaOH 16.5 g (0.413M) chloroform | | | |
| 4 | P | dimethylamine hydrochloride 8.16 g (100 mM) | 4.02 | 75 (58 | >99 |
| | | acetic acid 2.4 g (40 mM) | | HPLC) | Z:E |
| | | sodium triacetoxyborohydride 8.5 g (40 mM) | | | 100:0 |
| | | 3-(3-methoxyphenyl)-2-methylpent-2-enal | | | |
| | | 4.08 g (20 mM) [Z:E 99.7:0.3] | | | |
| | | NaOH 4.4 g (110 mM) chloroform | | | |
| 5 | P | dimethylamine hydrochloride 8.16 g (100 mM) | 4.25 | 79 (86 | >99 |
| | | acetic acid 2.4 g (40 mM) | | HPLC) | Z:E |
| | | sodium triacetoxyborohydride 8.5 g (40 mM) | | | 0:100 |
| | | 3-(3-methoxyphenyl)-2-methylpent-2-enal | | | |
| | | 4.08 g (20 mM) [Z:E 1:99] | | | |
| | | NaOH 4.4 g (110 mM) chloroform | | | |

### Synthesis of 2-bromo-N,N-dimethylpropanamide

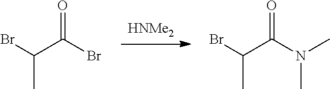

1 L 3-necked round bottomed flask was placed in ice bath and charged with 500 ml of chloroform. Dimethyl amine hydrochloride 37.8 g (1.5 eq.) and TEA 39.6 g (1.25 eq.) were added and mixing was continued until complete dissolution of hydrochloride. Flask was fitted with termomether and 100 ml dropping funnel. Funnel was charged with bromopropionyl bromide 67.65 g. Bromide was added dropwise while temperature was maintained below 10° C. When all bromide was added reaction was left mixing for 1 hour. Reaction mixture was transferred to 1 L flask and solvent was removed in vacuo. 250 ml of ethyl acetate was added and reaction mixture was transferred to separatory funnel. Organic phase was washed consequently: 3*150 ml 1M HCl, 3*25 ml water, 3*150 ml 1M NaHCO3, 3*25 ml water, 2*25 ml brine. Organic phase was dried over magnesium sulfate for 30 minutes. Solvent was removed in vacuo yielding 41.5 g (74%) of yellowish oil. Purity >99%.

| | | | Yield | Yield | Purity | NMR/ |
|---|---|---|---|---|---|---|
| No | Type | Conditions | (%) | (g) | (%) | LCMS |
| 1 | U | Scale 0.1 mol: | 85.5 | 15.4 | >99 | OK |
| | | dimethylamine hydrochloride 12.8 g (1.5 | | | (HPLC) | |
| | | eq.), 2-bromopropionyl bromide 21.6 g (1 | | | | |
| | | eq.), TEA 12.65 g (1.25 eq.) chloroform | | | | |
| | | 500 ml | | | | |
| 2 | P | Scale 0.31 mol: | 74 | 41.5 | >99 | OK |
| | | dimethylamine hydrochloride 37.8 g (1.5 | | | (HPLC) | |
| | | eq.), 2-bromopropionyl bromide 67.65 g | | | | |
| | | (1 eq.), TEA 39.6 g (1.25 eq.) chloroform | | | | |
| | | 500 ml | | | | |

### Reformatsky Reaction. Synthesis of 3-hydroxy-3-(3-methoxyphenyl)-N,N,2-trimethylpentanamide

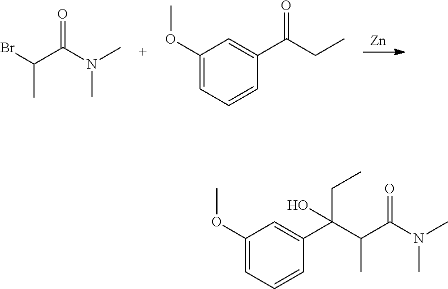

In 500 ml three necked flask equipped with condenser and dropping funnel zinc dust 14.66 g was placed. Magnetic bar was placed inside along with iodine crystal. Dust was mixed while heated with hot-gun. Heating was ceased when purple vapours of iodine started to form. When vapours condensed again in form of yellow film dropping funnel was charged with solution of 2-bromo-N,N-dimemethylpropanamide 34.44 g, 3-methoxyphenyl-ethylketone 33.46 g in 250 ml of THF. Solution was added dropwise on still hot zinc dust over 30 minutes. Whenever mixture was not boiling it was heated up again. Solution started to became white and milky. It forms foams when heated up to much. Gentle reflux was sustained overnight. The following day, mixture was decanted from remains of zinc. Solvent removed in vacuo. Crude product was purified on silica gel using mixture of hexanes:ethyl acetate 4:1 as eluent. Product with Rf=0.3 was gathered (one diastereomer), second Rf=0.2 was discarded. Reaction yielded 16.0 g (30%) of product with purity >99%.

### Dehydration of 3-hydroxy-3-(3-methoxyphenyl)-N,N,2-trimethylpentanamide

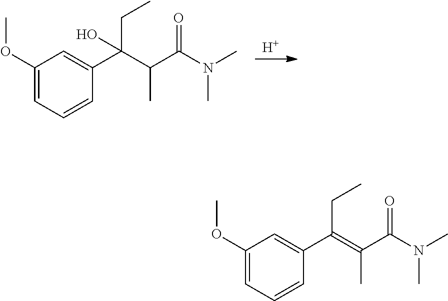

Reaction was up-scaled and 20 g of product was produced. After purification by liquid chromatography in eluent hexanes:ethyl acetate 1:6 pure compound was obtained and its structure confirmed. Four isomers of alkene were formed, two with “exo” C═C bond, and two with “endo”:

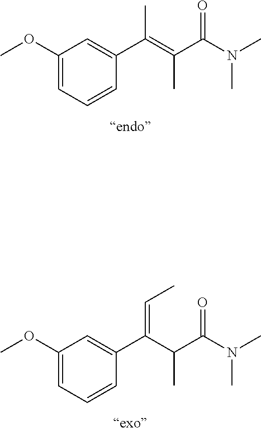

3-hydroxy-3-(3-methoxyphenyl)-N,N,2-trimethylpentanamide 27.0 g was placed in 500 ml flask fitted with Dean-Stark trap and condenser. 200 ml toluene were added along with drop of concentrated sulfuric acid. Heating was continued until no more water was condensing in trap. Organic phase was transferred to separatory funnel, washed with 3*20 ml 1 M NaHCO₃ 1*20 ml water. Organic phase was dried over magnesium sulfate for 30 minutes and solvent was evaporated. Process yielded 21.3 g (84%) of product with purity 97%.

### Reduction of 3-(3-methoxyphenyl)-N,N,2-trimethylpent-3-enamide

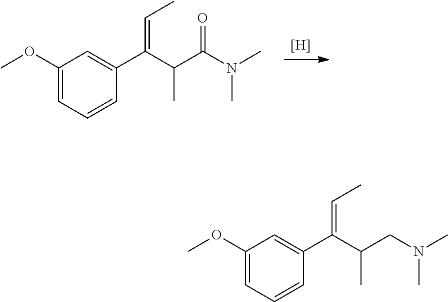

9.77 g of 3-(3-methoxyphenyl)-N,N,2-trimethylpent-3-enamine was dissolved in dry THF (200 ml). LiAlH₄ 1.65 g in pellets (ca. 0.5 g each pellet) was added in one portion. Immediately LAH starts to dissolve and gas evolution is observed. After 45 minutes 1.65 g of water was added, then 3.3 g of saturated solution of NaOH, and again 4.95 g of water. White pellets were formed in solution. Solution was filtered through Schott funnel. THF was removed in vacuo. 7.75 g of crude product was obtained which was purified on silica gel using mixture of hexane:EtOAc 1:5 as eluent. 5.92 g (64%) of product were obtained with purity 94%.

| No. | Type | Conditions | Amount | Purity | Yield | Notes |
|---|---|---|---|---|---|---|
| 1 | E | 3-(3-methoxyphenyl)- | 2.25 g | 95% | 75% | Full conversion. |
| | | N,N,2-trimethylpent- | | | | Side products found. |
| | | 3-enamine 3.21 g | | | | |
| | | LiAlH₄1.97 g (4 eq.) | | | | |
| | | r.t. overnight. | | | | |
| 2 | U | 3-(3-methoxyphenyl)- | 7.75 g crude | 94% | 64% | LC:EtOAc: n-hexane 5:1 |
| | | N,N,2-trimethylpent- | 5.92 g pure product | | | |
| | | 3-enamine 9.77 g | | | | |
| | | LiAlH₄1.65 g (1.1 eq) | | | | |
| | | 45 minutes. (mixture | | | | |
| | | of isomers used) | | | | |

### Hydrogenation; Synthesis of [3-(3-metoxyphenyl)-2-methylpentyl]-dimethylamine (According to WO 2008/01246)

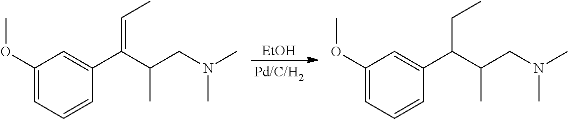 [SMILES: "EtOH" ; C/C=C(/c1cccc(OC)c1)C(C)CN(C)C ; CCC(c1cccc(OC)c1)C(C)CN(C)C]

| No | Type | Conditions | amount | purity | Yield |
|---|---|---|---|---|---|
| 1 | P. | 3-(3-methoxyphenyl)-N,N,2-trimethylpent-3-en-1- | 29.1 g | 93.7% | 96.0% |
| | | amine (30.52 g, 1.0 eq.), dissolved in EtOH (76 ml) | | | |
| | | and in Et₂O (4 ml). Pd/C (1.098 g) and HCl (3.5 ml, | | | |
| | | 0.302 eq.) were added. Reaction continued in parr | | | |
| | | apparatus overnight. pH₂ = 3.5 atm | | | |
| 2 | P. | 3-(3-methoxyphenyl)-N,N,2-trimethylpent-3-en-1- | 4.1 g | 94.9% | 78.5% |
| | | amine (5.58 g, 1.0 eq.), dissolved in EtOH (14 ml) | | | |
| | | and in Et₂O (1 ml). Pd/C (0.2007 g) and HCl (0.65 | | | |
| | | ml, 0.302 eq.) were added. Reaction continued in | | | |
| | | parr apparatus overnight. pH₂ = 3.5 atm | | | |

### Chiral Crystallization of OMe-Tapentadol—Examples

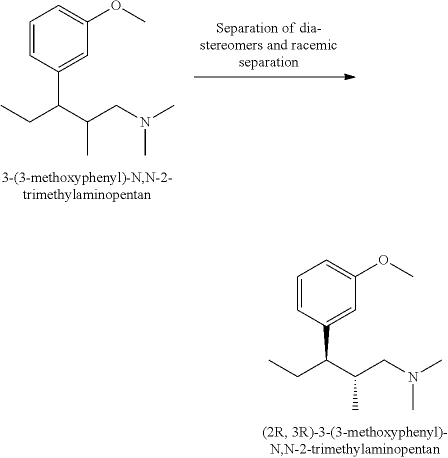 [SMILES: CCC(c1cccc(OC)c1)C(C)CN(C)C ; "Separation of dia-stereomers and racemicseparation" ; CC[C@@H](c1cccc(OC)c1)[C@@H](C)CN(C)C]

### Method A. (One Equivalent of Chiral Acid).

0.100 g (0.42 mmol) of O-methyl-Tapentadol was placed in a vial and dissolved in 1 ml of ethyl acetate. 0.153 g (0.42 mmol) of (−)DBTA was placed in a vial and dissolved in 1 ml of ethyl acetate. Solution of (−)DBTA was placed in a syringe and added dropwise to the solution of O methyl-Tapentadol. Immediately crystals started to form. The vial was kept at room temperature for 24 h. Solid residue was filtered off and analyzed.

### Method B. (One to 0.4 Equivalent of Chiral Acid. 0.6 Equivalent of Achiral Acid).

0.100 g (0.42 mmol) of O-methyl-Tapentadol was placed in a vial and dissolved in 1 ml of ethyl acetate. 0.060 g (0.168 mmol) of (−) DBTA was placed in a vial and dissolved in 1 ml of ethyl acetate. 0.08 ml of 3.1 N solution of HCl in ethyl acetate was added to the solution of (−) DBTA. Solution of (−) DBTA and HCl was placed in a syringe and added dropwise to the solution of O-methyl-Tapentadol. Immediately crystals started to form. Vial was kept at room temperature for 24 h. Solid residue was filtered off and analyzed.

### Method C. (One to 0.4 Equivalent of Chiral Acid).

0.100 g (0.42 mmol) of O-methyl-Tapentadol was placed in a vial and dissolved in 0.5 ml of ethyl acetate. 0.060 g (0.168 mmol) of (−) DBTA was placed in a vial and dissolved in 0.5 ml of ethyl acetate. Solution of (−) DBTA was placed in a syringe and added dropwise to the solution of O-methyl-Tapentadol. Immediately crystals started to form. The vial was kept at room temperature for 24 h. Solid residue was filtered off and analyzed.

All O-methyl-Tapentadol chiral resolution analyses were performed on either HPLC Agilent 1200 or HPLC Dionex Ultimate 3000 using Astec Chirobiotic V2 4.6/250 mm 5 micrometer column. Mobile phase 98.5% MeOH. 0.1% AcOH. 0.05% NH3*H2O (28-30% solution). flow 0.4 ml. time 40 min. 10° C.

### Example 1

Crystallization was performed according to method A. 1.09 g of O-methyl-Tapentadol. 1.66 g of (−)DBTA and 50 ml of ethyl acetate was used. Crystallization started at 50° C. and was slowly cooled down to 20° C. and kept at this temperature for 24 hours. Crystals were filtered, collected and analyzed. Process yielded 1.02 g of salt. Isomers ratio (S,R+R,R):(R,S):allylic amine:(S,S) according to chiral HPLC 74:17:3:6.

### Example 2

Crystallization was performed according to method B. 0.380 g of O-methyl-Tapentadol from example 1. 0.425 g of (−)DBTA (0.74 eq.) and 10 ml of ethyl acetate consisting 0.43 mmol of HCl was used. Crystallization started at 50° C. and was slowly cooled down to 20° C. and kept at this temperature for 24 hours. Crystals were filtered, collected and analyzed. Process yielded 0.5755 g. Isomers ratio (S,R+R,R):(R,S):allylic amine:(S,S) according to chiral HPLC 94.5:3:1.5:1.

### Example 3

Crystallization was performed according to method C. 0.139 g of O-methyl-Tapentadol from example 2 0.190 g of (−)DBTA (0.9 eq.) and 5 ml of ethyl acetate was used. Crystallization started at 50° C. and was slowly cooled down to 20° C. and kept at this temperature for 24 hours. Crystals were filtered, collected and analysed. Process yielded 0.219 g of salt. Isomers ratio (%): (R,R):(S,R):(S,S):(R,S)=99.5:0:0:0.5 (according to chiral HPLC).

### Example 4

Crystallization was performed according to method A. 0.3 g (1.26 mmol) of O-methyl-Tapentadol. 0.459 g (1.26 mmol) of (−)DBTA and 25 ml of ethyl acetate was used. Crystallization started at 20° C. and was kept at 20° C. for 24 hours. Crystals were filtered, collected and analyzed. Process yielded 0.1655 g of salt and 0.071 g and amine (after liberation). Isomers ratio (S,R+R,R):(R,S):allylic amine:(S,S) according to chiral HPLC 63.48:9.8:14.4:3.73.

### Example 5

Crystallization was performed according to method B. 0.071 g (0.3 mmol) of O-methyl-Tapentadol from example 4. 0.068 g (0.19 mmol) of (−)DBTA and 1 ml of ethyl acetate consisting 0.11 mmol of HCl. Crystallization started at 20° C. and was kept at 20° C. for 24 hours. Crystals were filtered, collected and analysed. Process yielded 0.0163 g. Isomers ratio (R,R):(S,R):(S,S):(R,S)=93.9:2.3:2.9:0.9 (according to chiral HPLC).

### Deprotection, Synthesis Tapentadol

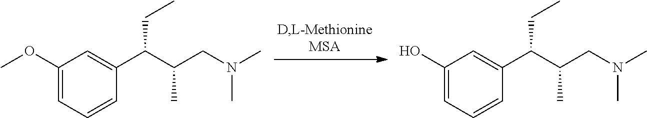

| | | | Yield | |
|---|---|---|---|---|
| Nr | Scale | Conditions | (%) | Notes |
| 1 | 2 mmol | 3-(3-methoxyphenyl)-2methyl-pentyl-dimethylamine | 0.47 g | NMR |
| | | hydrochloride 0.55 g mixture of isomers (S,R) + (R,R) 3:1 | | OK |
| | | D,L-Methionin 0.37 g Methanesulfuric acid 2.15 ml 14 | | |
| | | hours in 80° C. Crystallization IPrOH:water | | |

Reaction mixture was dissolved in isopropanol and 1 drop of water was added. The reaction mixture was stirred and heated in 80° C. for 15 minutes. Reaction mixture was cooled down slowly. Crystalline was filtered.

### SHORT DESCRIPTION OF THE FIGURES

FIG. 1 and FIG. 2 show preferred synthesis schemes according to the invention.

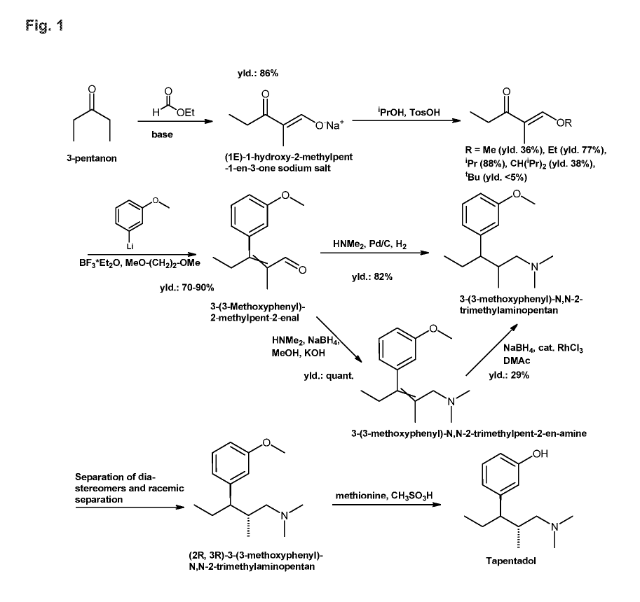

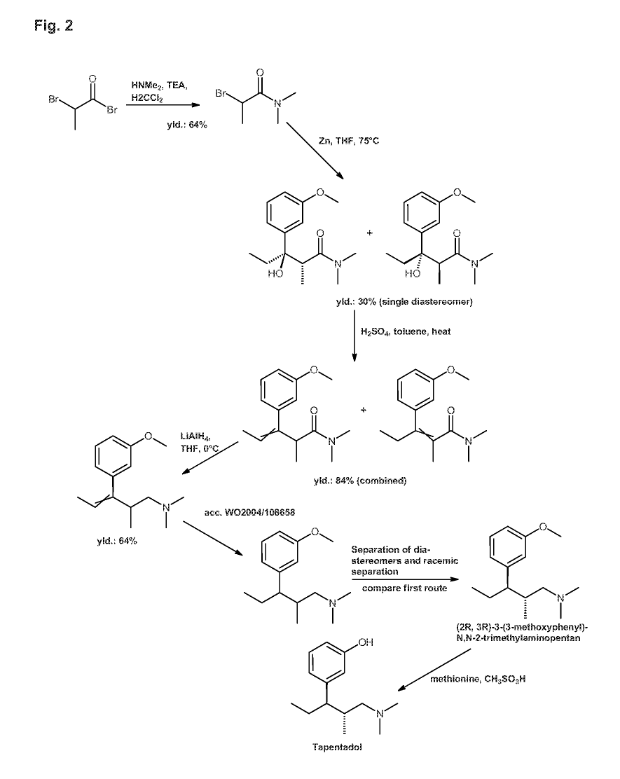

### CITED PRIOR ART

WO 2008/012047
EP 0 693 475 A
WO 2004/108658
WO 2011/067714
WO 2011/080736
WO 2008/01246

1. A process for the preparation of a compound of formula (I) 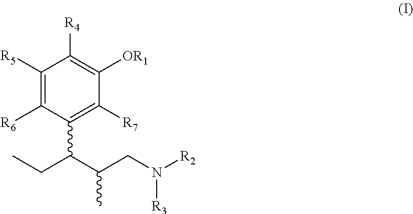
the process comprising
(a1) providing a compound of formula (II) 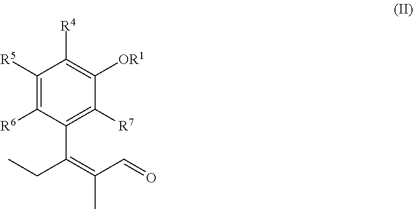
(a2) reacting the compound of formula (II) with an amine HN(R²)(R³) and reducing the resulting reaction product with hydrogen in the presence of a catalyst,
to give the compound of formula (I).

2. The process of claim 1 comprising
(a1) providing a compound of formula (II) 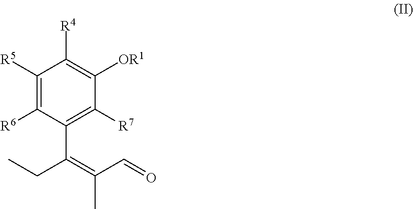
(a2) reacting the compound of formula (II) with HN(R²)(R³)) in the presence of a reducing agent, thereby forming a compound of formula (III) 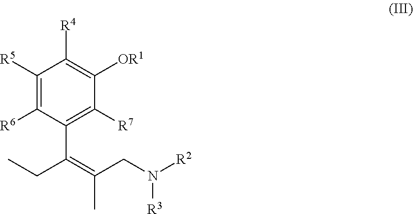
(a3) optionally isolating the compound of formula (III),
(a4) reducing the compound of formula (III) with hydrogen in the presence of a catalyst,
to give the compound of formula (I).

3. A process for the preparation of a compound of formula (I) 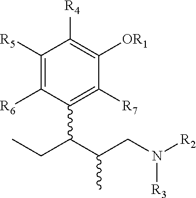
the process comprising
(aa) providing a compound of formula (IX) 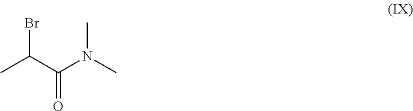
(ab) providing a compound of formula (X) 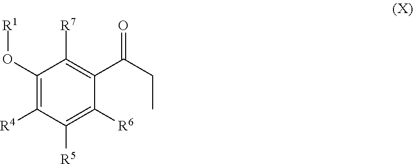
(ac) reacting the compound of formula (IX) with the compound of formula (X) in the presence of Zn to give a compound of formula (XI) 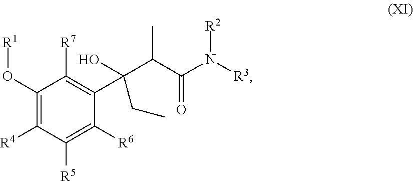
(ad) dehydrating the compound of formula (XI) to give a compound of formula (XIIa) 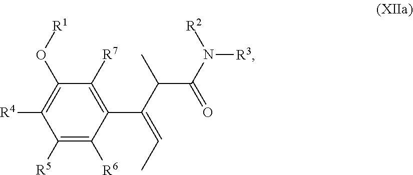
and/or a compound of formula (XIIb) 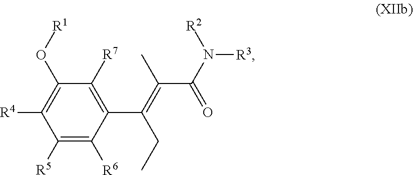
(ae) reducing the compound of formula (XIIa) and/or (XIIb),
to give the compound of formula (I).

4-5. (canceled)

6. The process according to claim 1, wherein compound of formula (I) 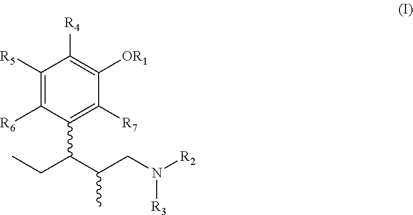
comprises a diastereomeric mixture of the compounds of formula (Ia) and formula (Ib)

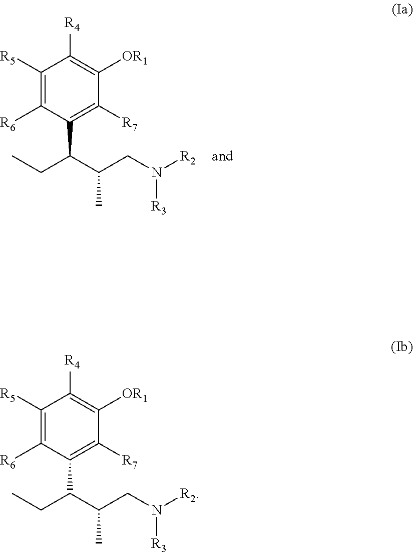

7. The process of claim 1
wherein the process further comprises
(a) providing a compound of formula (I) 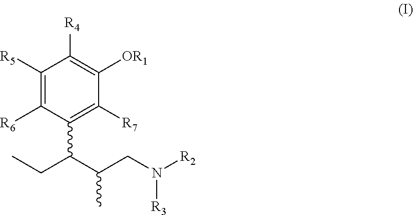
comprising a diastereomeric mixture of the compounds of formula (Ia) and formula (Ib) 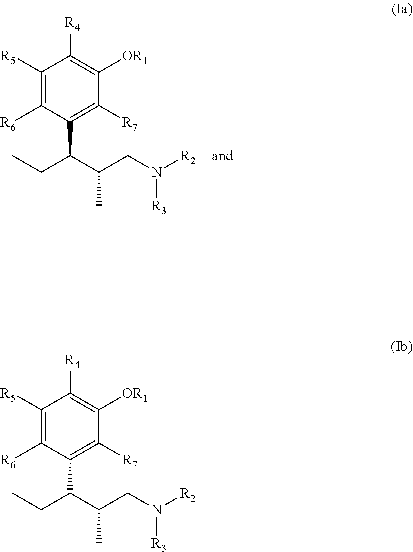
wherein the providing is by a process according to claim 1,
(b) forming an acid salt (T*) of at least part of the compound of formula (I) by treating the compound of formula (I) with a chiral acid in a suitable solvent, and precipitating at least part of the acid salt (T*) formed, thereby obtaining a mixture comprising the precipitated acid salt (T) and the solvent;
(c) optionally separating the precipitated acid salt (T) of the compound of formula (I) from the mixture obtained in (b),
wherein the acid salt (T) of the compound of formula (I) contains at least 90% by weight of the acid salt of the compound of formula (Ia) based on the total weight of the acid salt (T) of the compound of formula (I),

8-9. (canceled)

10. The process of claim 1, further comprising
(i) providing an acid salt (T) of a compound of formula (I) wherein the acid salt (T) is prepared by a process according to claim 7
(ii) (ii) transforming the group —OR¹ to OH,
(iii) optionally purifying the compound obtained in (ii)
to give the compound of formula (XIV) 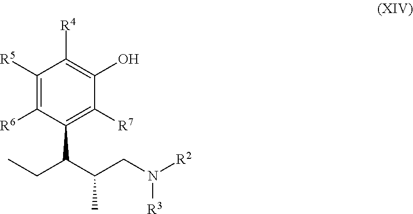
(iv) optionally preparing pharmaceutically acceptable salt or solvate of compound (XIV) of (ii) or (iii).

11-15. (canceled)

16. A compound of formula (II) 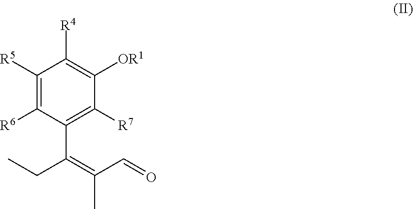
wherein R¹ is selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl.

17. A compound of formula (III) 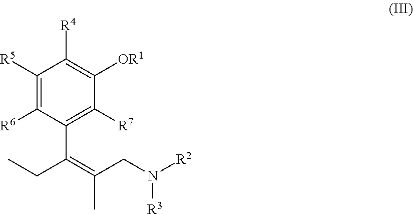
wherein R¹ is selected from the group consisting of alkyl, aryl, cycloalkyl, heteroalkyl, heteroaryl and heterocycloalkyl,
R² and R³, are, independently of each other, selected from the group consisting of alkyl, aryl, cycloalkyl, heteroalkyl, heteroaryl and heterocycloalkyl,
R⁴, R⁵, R⁶ and R⁷, are independently of each other, selected from the group consisting of H, alkyl, aryl, cycloalkyl, heteroalkyl, heteroaryl and heterocycloalkyl.

18. A compound of formula (IV) 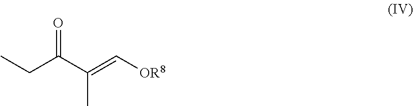
wherein R⁸ is —CH(isopropyl)₂.

19-20. (canceled)
